(19) United States
(12) Patent Application Publication
(10) Pub. No.: US 2017/0211063 A1
(43) Pub. Date: Jul. 27, 2017

(54) METHOD FOR IMPROVED HIGH-LEVEL SECRETORY PRODUCTION OF PROTEIN

(71) Applicant: Daiichi Sankyo Company, Limited, Tokyo (JP)

(72) Inventors: Koichi NONAKA, Oura-gun (JP); Takeshi SUZUKI, Oura-gun (JP); Masashi TSUDA, Oura-gun (JP); Satoshi BABA, Tokyo (JP); Kimihisa ICHIKAWA, Oura-gun (JP); Yasunori CHIBA, Tsukuba-shi (JP); Takehiko YOKO-O, Tsukuba-shi (JP); Rie ITO, Tsukuba-shi (JP)

(73) Assignee: Daiichi Sankyo Company, Limited, Tokyo (JP)

(21) Appl. No.: 15/329,636

(22) PCT Filed: Jul. 29, 2015
(86) PCT No.: PCT/JP2015/071503

§ 371 Date: Jan. 27, 2017

(30) Foreign Application Priority Data
Jul. 30, 2014 (JP) 2014-155272

(51) Int. Cl. C12N 15/113; C12N 15/62; C12N 15/81

(52) U.S. Cl. CPC C12N 15/113; C12N 15/81; C12N 15/62

(57) ABSTRACT

The object of the present invention is to provide a production system that is capable of high-level secretory production of a protein (and in particular, a protein with a complicated structure such as a structure with S—S bonds) in a host cell such as yeast and is suitable for industrial production with high safety that does not require explosion-proof facilities. The present invention provides a transformed yeast into which a chaperone gene has been introduced and in which the aox1 gene and/or the protease gene have been disrupted and a method for producing a protein involving the use of such transformed yeast.

### TECHNICAL FIELD

The present invention relates to a method for high-level secretory production of a protein in yeast.

### BACKGROUND ART

The market for protein pharmaceuticals such as therapeutic proteins and antibody drugs is rapidly expanding due to the development of genetic engineering techniques. Animal cells such as CHO or NSO, insects such as silkworms, insect cells such as SF9, and microorganisms such as E. coli or yeast have been used as hosts in which protein pharmaceuticals are to be produced. In particular, yeast systems are capable of high-density culture and thus they are extensively used as systems that are capable of secretory production of useful proteins in relatively inexpensive media.

Secretory proteins pass through the translocon and enter into the endoplasmic reticulum when the amino acid regions of signal sequences at their N terminuses are recognized by signal recognition particles (SRPs). When secretory proteins pass through the translocon, the higher-order structures thereof are loosened, and the proteins are folded in the endoplasmic reticulum. While secretory protein folding is able to spontaneously occur, various molecular chaperones assist such folding. A native conformation formed in the endoplasmic reticulum is critical for secretion, and misfolded proteins cannot enter the secretory pathway located downstream. Thus, proteins having abnormal higher-order structures are disadvantageously accumulated therein. Such disturbance in modification that takes place in the endoplasmic reticulum (i.e., addition of a sugar chain or a disulfide bond) and deteriorated transportation from the endoplasmic reticulum causes “endoplasmic reticulum stress.” As a means for dealing with such endoplasmic reticulum stress, a stress response referred to as “unfolded protein response (UPR)” is induced in eukaryotic cells. Transcription induction and translation regulation of UPR are responses that restore accumulated abnormal proteins. There is also a mechanism referred to as “ER-associated degradation (ERAD)” that degrades and eliminates abnormal proteins so as to maintain homeostasis in the endoplasmic reticulum. Further, molecular chaperones that loosen the aggregated proteins for the purpose of folding are known, as are molecular chaperones that assist protein folding in the endoplasmic reticulum. For example, HSP104 can perform a reaction that cannot be performed with the aid of other chaperones that cooperate with HSP70 and solubilizes proteins from the aggregates (Non-Patent Document 1).

Meanwhile, a variety of interactions, such as hydrogen bonds, electrostatic interactions, and hydrophobic interactions, occur between amino acids inside a protein steric structure. In particular, covalent bonds between sulfur atoms that are formed upon two-electron oxidation of two cysteines (which are referred to as “disulfide bonds”) play very important roles in stabilizing protein steric structure because of their strong properties. In fact, many secretory proteins that are secreted extracellularly have disulfide bonds. This is presumed to be the case because of the necessity of strengthening of protein structure, so that a protein can function outside a cell, where the environment is physically and chemically more severe than that in the environment inside the cell, which is enveloped by a membrane. In the case of eukaryotic cells such as yeast cells, introduction of a disulfide bond via protein oxidative folding is carried out by the oxidative protein disulfide isomerase (PDI) in the endoplasmic reticulum (Non-Patent Document 2). PDI that is reduced via oxidization of substrate proteins is reoxidized by oxidative ERO1 localized in the vicinity of the membrane (Non-Patent Documents 3 and 4). In yeast endoplasmic reticulum, there are 5 types of PDI families (i.e., PDI1, EUG1, MPD1, MPD2, and EPS1) (Non-Patent Document 5). Among such PDI families, those that are confirmed to form an intramolecular disulfide bond with ERO1 are limited to PDI1 and MPD2. It is also reported that the efficiency of protein oxidative folding is improved with BiP/Kar2, which functions in conjunction with PDI (Non-Patent Document 6). BiP/Kar2 is also associated with induction by active HAC1 of various genes associated with the aforementioned UPR. Active HAC1 is activated by the splicing of HAC1 via the IRE1 transmembrane kinase/nuclease. IRE1 to which BiP/Kar2 is bound is dissociated when BiP/Kar2 acts on a protein having an abnormal structure in the endoplasmic reticulum, it exhibits nuclease activity through the formation of a dimmer, and it produces active HAC1 through the splicing of HAC1 (Non-Patent Documents 7 and 8). Also, Bip/Kar2 is associated with protein folding in the endoplasmic reticulum in conjunction with SCJ1 located in the endoplasmic reticulum (Non-Patent Document 9).

Thus, it has been demonstrated that various molecular chaperones are associated with the correct folding of secretory proteins. It has been reported that one or more types of genes encoding molecular chaperone proteins, such as PDI1, ERO1, or Kar2, are co-expressed in the presence of a target protein to be expressed in yeast, so as to improve the secretory productivity of a target protein having a complicated steric structure (Patent Document 1).

Even if the productivity of target protein secretion into media is improved with coexpression of genes encoding chaperone proteins, such as PDI1, ERO1, or Kar2, some target proteins may occasionally rapidly degrade in media. In particular, a protease existing in a vacuole that is known as a protein-degrading organelle of yeast is reported to be associated with secretory protein degradation (Non-Patent Document 10). Many proteolytic enzymes are present in vacuoles, such as vacuolar trehalase, aminopeptidase I, vacuolar alkaline phosphatase, and vacuolar RNase, in addition to proteinase A, proteinase B, and carboxypeptidase Y, and activity thereof is regulated such that it is exerted in vacuoles. In particular, proteinase A and proteinase B function as key proteases that activate themselves or carboxypeptidase Y, and they play key roles in a proteolytic system (Non-Patent Documents 11 and 12). It has been reported that an acidic protease (i.e., proteinase A) exerts strong activity under acidic conditions, but such activity is attenuated as pH increases (Non-Patent Document 13). Thus, a culture method in which the pH of a culture medium is adjusted so as to inhibit protease activity has been studied, although such method may affect the proliferation of host cells.

In methhylotrophic yeast, methanol metabolism is initiated upon oxidation of methanol by alcohol oxidase (AOX), the generated formaldehyde is fixed to xylose 5-phosphate with the aid of a dihydroxyacetone synthase (DAS), it is used as a cell constituent in a glycolysis system, and it is also oxidized to CO₂ with the aid of glutathione-dependent formaldehyde dehydrogenase (FLD) and formate dehydrogenase (FDH) in the cytoplasm (Non-Patent Document 14). Many gene promoters encoding enzymes associated with methanol metabolism, such as pmp20- and pmp47-promoters, have been known as gene promoters the expression of which is regulated by methanol, and examples thereof include alcohol oxidase (aox1, aox2) promoter, dihydroxyacetone synthase (das1) promoter, formate dehydrogenase (fdh1) promoter, and methanol oxidase (mox) promoter (Non-Patent Document 15). Promoters that regulate the expression of enzymes associated with methanol metabolism are very strong. Thus, such promoters are generally used to achieve secretory production of various target proteins in methhylotrophic yeast. In particular, an aox1 promoter of Pichia pastoris is known as a very strong promoter induced by methanol.

When target proteins are to be secreted and produced under the control of a promoter that regulates the expression of enzymes associated with methanol metabolism, methanol induction is considered to be necessary. Methanol is a deleterious substance classified as a Class 2 Flammable Liquid, the use thereof in an amount exceeding the designated level is regulated under the Fire Defense Law, and explosion-proof factories and facilities are required. If secretory production of target proteins equivalent to that induced to express with the aid of a large quantity of methanol can be achieved with the use of methanol in as small an amount as possible, accordingly, industrial values thereof are significant. In addition, various positive transcription factors used as methanol-metabolizing enzyme promoters of methhylotrophic yeast are used to induce the expression of methanol-metabolizing enzymes inherent to yeast, as well as the expression of target proteins that have been newly introduced. In fact, expression of various oxidases, such as D-amino acid oxidase, fructosyl amino acid oxidase, and peroxisome/acetyl spermidine oxidase, in addition to the hepatitis B surface antigen gene, has been attempted under the control of the aox1 promoter with the use of a methhylotrophic yeast (Candida boidinii) in which the aox1 gene inherent thereto has been disrupted, and the target protein expression level is enhanced in a strain in which the aox1 gene inherent thereto has been disrupted, compared with the original parent strain (Patent Document 2 and Non-Patent Documents 16, 17, and 18).

As described above, the use of a wide variety of methods has been demonstrated regarding high-level secretory expression of proteins in yeast. When host cells are transformed via gene introduction, gene disruption, or other means, in general, cells receive some stress. Thus, synergistic or additive effects cannot always be attained merely by employing several conventional techniques in combination. In addition, there have been no reports concerning high-level secretory expression of target proteins with the use of the various chaperones in combination with the various techniques described above.

### PRIOR ART DOCUMENTS

### Patent Documents

[Patent Document 1] WO 2009/057813
[Patent Document 2] JP S62-104585 A (1987)

### Non-Patent Document

[Non-Patent Document 1] Glover J R, Lindquist S, Hsp104, Hsp70, and Hsp40: A novel chaperone system that rescues previously aggregated proteins. Cell (1998) 94: 73-82
[Non-Patent Document 2] Benjamin P. Tu and Jonathan S. Weissman, Oxidative protein folding in eukaryotes: mechanisms and consequences. J. Cell Biol. (2004) 164:341-346
[Non-Patent Document 3] Mezghrani, A., Fassio, A., Benham, A., Simmen, T., Braakman, I., and Sitia, R., Manipulationof oxidative proteinfolding and PDlredox state inmammalian cells. EMBO. J. (2001) 20: 6288-6296
[Non-Patent Document 4] Frand, A. R. and C. A. Kaiser, Ero1p oxidizes protein disulfide isomerase in a pathway for disulfide bond formation in the endoplasmic reticulum. Mol. Cell (1999) 4:469-477
[Non-Patent Document 5] Per Norgaard, Vibeke Westphal, Christine Tachibana, Lene Alsoe, Bjorn Holst, Jakob R. Winther, Functional Differences in Yeast Protein Disulfide Isomerases. J. Cell Biology (2001) 152(3): 553-562,
[Non-Patent Document 6] Marcus Mayer, Ursula Kies, Robert Kammermeier, and Johannes Buchner, BiP and PDI Cooperate in the Oxidative Folding of Antibodies in Vitro, J. Biol. Chem. (2000) 275(38): 29421-29425.
[Non-Patent Document 7] Cox JS., Shamu CE., Walter P., Transcriptional induction of genes encoding endoplasmic reticulum resident proteins requires a transmembrane protein kinase. Cell (1993) 73:1197-1206
[Non-Patent Document 8] Sidrauski C. and Walter P., The transmembrane kinase Ire1p is a site-specific endonuclease that initiates mRNA splicing in the unfolded protein response. Cell (1997) 90(6): 1031-1039
[Non-Patent Document 9] Susana Silberstein, Gabriel Schlenstedt, Pam A. Silver, and Reid Gilmore, A Role for the DnaJ Homologue Scj1p in Protein Folding in the Yeast Endoplasmic Reticulum. J. Cell Biol. (1998) 143(4): 921-933
[Non-Patent Document 10] Morozkina E V, Marchenko A N, Keruchenko J S, Keruchenko I D, Khotchenkov V P, Popov V O, and Benevolensky S V, Proteinase B disruption is required for high level production of human mechano-growth factor in Saccharomyces cerevisiae. J. Mol. Microbiol. Biotechnol. (2010) 18(3): 188-194
[Non-Patent Document 11] H. Bart van den HAZEL, Morten C. KIELLAND-BRANDT, and Jakob R. WINTHER, Autoactivation of proteinase A initiates activation of yeast vacuolar zymogens. Eur. J. Biochem. (1992) 207: 277-283
[Non-Patent Document 12] Vicki L. Nebes and Elizabeth W. Jones, Activation of the proteinase B precursor of the yeast Saccharomyces cerevisiae by autocatalysis and by an internal sequence. J. Biol. Chem. (1991) 266(34): 22851-22857
[Non-Patent Document 13] Susanne O. SORENSEN, H. Bart VAN DEN HAZEL, Morten C. KIELLAND-BRANDT, and Jakob R. WINTHER, pH-dependent processing of yeast procarboxypeptidase Y by proteinase A in vivo and in vitro. Eur. J. Biochem. (1994) 220: 19-27
[Non-Patent Document 14] Ida J. van der Kleia, Yurimotob H, Sakaib Y, Venhuisa M, The significance of peroxisomes in methanol metabolism in methylotrophic yeast. Biochim. Biophys. Acta. (2006) 1763: 1453-1462
[Non-Patent Document 15] Yurimoto H, Komeda T, Lim C R, Nakagawa T, Kato N, Sakai Y, Regulation and evaluation of five methanol-inducible promoters in methylotrophic yeast Candida boidinii. Biochim. Biophys. Acta. (2000) 1493(1-2): 56-63
[Non-Patent Document 16] Yurimoto H, Hasegawa T, Sakai Y, Kato N, Characterization and High-level production of D-amino acid oxidase in Candida boidinii. Biosci. Biotechnol. Biochem. (2001) 65(3), 627-633
[Non-Patent Document 17] Sakai Y, Yoshida H, Yurimoto H, Yoshida N, Fukuya H, Takabe K, Kato N, Production of fungal fructosyl amino acid oxidase useful for diabetic diagnosis in the peroxisome of Candida boidnii. FEBS Lett. (1999) 459, 233-237
[Non-Patent Document 18] Nishikawa M, Hagishita T, Yurimoto H, Kato N, Sakai Y, Hatanaka T, Primary structure and expression of peroxisomal acetylspermidine oxidase in the methylotrophic yeast Candida boidinii. FEBS Lett. (2000) 476, 150-154

### SUMMARY OF THE INVENTION

### Problems to be Solved by the Invention

It is an object of the present invention to provide a production system that is capable of high-level secretory production of a protein (and in particular, a protein with a complicated structure, such as a structure with S—S bonds) in a host cell such as yeast, is suitable for industrial production with high safety, and does not require explosion-proof facilities.

### Means for Solving the Problem

The present inventors have conducted concentrated studies in order to attain the above objects. As a result, they discovered that a yeast strain into which a chaperone gene has been introduced and in which the aox1 gene encoding alcohol oxidase has been disrupted may be used, so that high-level secretory production of a target protein induced by low-concentration methanol would become possible under the control of the aox1 promoter. The present inventors also discovered that acidic proteases, such as proteinase B (PRB1) and proteinase A (PEP4), were significantly associated with degradation of the target protein expressed in yeast, and they confirmed that regulation of the pH level of a medium aimed at disruption of the prb1 gene encoding proteinase B and/or suppression of activity of acidic protease such as proteinase A in yeast would lead to significant improvement in the secretory production amount of the target protein.

The high-level protein secretory production system comprising the above described features in combination enables a significant reduction in the amount of methanol to be added. Thus, such system can be used as a highly safe production system that is suitable for industrial production. The present invention has been completed on the basis of such findings.

Specifically, the present invention includes the following.

(1) A transformed yeast into which a chaperone gene has been introduced and in which the aox1 gene has been disrupted.
(2) The transformed yeast according to (1), wherein the chaperone gene is at least one gene selected from the group consisting of genes (a) to (d) below:

(a) a gene encoding PDI1, ERO1, Kar2, MPD1, SCJ1, EUG1, or HSP104 derived from Ogataea minuta (O. minuta);

(b) a gene encoding PDI1, MPD1, SCJ1, ERO1, FKB2, JEM1, LHS1, MPD2, ERJ5, or EUG1 derived from Saccharomyces cerevisiae (S. cerevisia);

(c) a gene encoding PDI, ERO1-Lα, ERO1-Lβ, or GRP78 derived from a human; and

(d) a gene exhibiting 95% or higher sequence homology to a base sequence of any of the genes (a) to (c).

(3) The transformed yeast according to (1), wherein the chaperone gene is at least one gene selected from the group consisting of genes (a) to (g) below:

(a) a gene encoding PDI1 derived from O. minuta;

(b) a gene encoding ERO1 derived from O. minuta;

(c) a gene encoding Kar2 derived from O. minuta;

(d) a gene encoding PDI1 derived from S. cerevisiae;

(e) a gene encoding PDI derived from a human;

(f) a gene encoding ERO1 derived from a human; and

(g) a gene exhibiting 95% or higher sequence homology to a base sequence of any of the genes (a) to (f).

(4) The transformed yeast according to (1), wherein the chaperone gene is any of the chaperone genes (a) to (g) below:

(a) a combination of a gene encoding PDI1, a gene encoding ERO1, and a gene encoding Kar2 derived from O. minuta;

(b) a combination of a gene encoding PDI1 and a gene encoding Kar2 derived from O. minuta;

(c) a combination of a gene encoding PDI derived from a human and a gene encoding ERO1 derived from O. minuta;

(d) a combination of a gene encoding PDI1 and a gene encoding ERO1 derived from O. minuta;

(e) a combination of a gene encoding PDI derived from a human, a gene encoding ERO1-Lβ derived from a human, and a gene encoding GRP78 derived from a human;

(f) a combination of a gene encoding PDI derived from a human, a gene encoding ERO1 derived from O. minuta, and a gene encoding GRP78 derived from a human; and

(g) a gene exhibiting 95% or higher sequence homology to a base sequence of any of the genes (a) to (f).

(5) The transformed yeast according to any of (1) to (4), wherein the protease gene has been disrupted.
(6) The transformed yeast according to (5), wherein the protease is a prb1 gene.
(7) A transformed yeast into which a chaperone gene has been introduced and in which a protease gene has been disrupted.
(8) The transformed yeast according to (7), wherein the protease is a prb1 gene.
(9) The transformed yeast according to any of (1) to (8), wherein the yeast is a methhylotrophic yeast.
(10) The transformed yeast according to any of (1) to (9), which comprises a gene encoding a target protein introduced thereinto.
(11) Use of the transformed yeast according to any of (1) to (10) for the production of a target protein.
(12) A method for producing a protein comprising culturing the transformed yeast according to (10) in a medium and sampling a target protein from the culture product.
(13) The method for producing a protein according to (12), wherein culture is conducted under conditions in which protease activity is inhibited.
(14) The method for producing a protein according to (12) or (13), wherein culture is conducted in a medium with a pH of 6.0 to 7.5.
(15) The method for producing a protein according to any of (12) to (14), wherein a nitrogen source is added to the medium.
(16) The method for producing a protein according to any of (12) to (15), wherein the amount of methanol added to the medium is 2% (v/v) or less.
(17) A target protein produced by the method according to any of (12) to (16).
(18) A method for producing a transformed yeast comprising step (i) in addition to either or both step (ii) and/or (iii):

(i) a step of introducing a chaperone gene into yeast; and

(ii) a step of disrupting the aox1 gene in yeast; and/or

(iii) a step of disrupting the prb1 gene in yeast.

(19) The method of production according to (18), which further comprises a step of introducing a gene encoding a target protein.

### Effects of the Invention

The present invention enables high-level secretory production of a protein having a complicated structure, such as a structure with S—S bonds, as well as a normal protein, in a correctly folded form in a transformed yeast resulting from the introduction of a chaperone gene, the disruption of the aox1 gene, and/or the disruption of the protease gene. In addition, long-term culture (mass production) can be performed by culturing such transformed yeast under conditions in which protease activity is inhibited. In the protein production system involving the use of the transformed yeast according to the present invention, the amount of methanol used can be reduced to a significant extent. Thus, such system can be used as a highly safe protein production system that is suitable for industrial production (mass production).

This patent application claims priority from Japanese Patent Application No. 2014-155272 filed on Jul. 30, 2014, and it includes part or all of the contents as disclosed in the description thereof.

### BRIEF DESCRIPTION OF THE DRAWINGS

FIG. 1 shows a method for producing a strain in which the ura3 gene has been disrupted.

FIG. 2 shows a method for producing a strain in which the aox1 gene has been disrupted.

FIG. 3 shows the structure of a chaperone gene expression vector (onaP11007: OmPDI1+OmERO1+OmKar2 expression vector).

FIG. 4 shows a method for producing a strain in which the prb1 gene has been interrupted.

FIG. 5 shows a method for producing a kex2 expression plasmid (kex2 expression plasmid: pOMEA-Z1-KEX2, kex2 expression cassette).

FIG. 6-1 shows a comparison of the secretory production amount of a KEX2 protein induced by methanol (1: the NBRC 10746 strain into which a chaperone (OmPDI1/OmERO1/OmKar2) has been introduced (a KEX2-producing strain derived from the NBRC10746+PEK strain); 2: a strain into which a chaperone (OmPDI1/OmERO1/OmKar2) has been introduced and in which the aox1 gene has been disrupted (a KEX2-producing strain derived from the Δaox1+PEK strain); 3: a strain into which a chaperone (OmPDI1/OmERO1/OmKar2) has been introduced and in which the prb1 gene has been interrupted (a KEX2-producing strain derived from the NBRC10746+PEK dprb1 strain); and 4: a strain in which the prb1 gene has been interrupted, into which a chaperone (OmPDI1/OmERO1/OmKar2) has been introduced, and in which the aox1 gene has been disrupted (a KEX2-producing strain derived from the Δaox1+PEK dprb1 strain).

FIG. 6-2 shows a comparison of enzymatic activity of the KEX2 protein (1: the NBRC 10746 into which a chaperone (OmPDI1/OmERO1/OmKar2) has been introduced (the KEX2-producing strain derived from the NBRC10746+PEK strain); 2: a strain into which a chaperone (OmPDI1/OmERO1/OmKar2) has been introduced and in which the aox1 gene has been disrupted (the KEX2-producing strain derived from the Δaox1+PEK strain); 3: a strain into which a chaperone (OmPDI1/OmERO1/OmKar2) has been introduced and in which the prb1 gene has been interrupted (the KEX2-producing strain derived from the NBRC10746+PEK dprb1 strain); and 4: a strain in which the prb1 gene has been interrupted, into which a chaperone (OmPDI1/OmERO1/OmKar2) has been introduced, and in which the aox1 gene has been disrupted (the KEX2-producing strain derived from the Δaox1+PEK dprb1 strain); (white bar: third quartile-median; black bar: median-first quartile).

FIG. 7 shows a method for producing the hsa expression plasmid (hsa expression plasmid: pOMEA-Z1-HSA; hsa gene expression cassette).

FIG. 8 shows the secretory production amount of the HSA protein induced by methanol (deep well plate scale) (1: the NBRC 10746 strain into which a chaperone (OmPDI1/OmERO1/OmKar2) has been introduced (the HSA-producing strain derived from the NBRC10746+PEK strain); 2: a strain into which a chaperone (OmPDI1/OmERO1/OmKar2) has been introduced and in which the aox1 gene has been disrupted (the HSA-producing strain derived from the Δaox1+PEK strain); 3: a strain into which a chaperone (OmPDI1/OmERO1/OmKar2) has been introduced and in which the prb1 gene has been interrupted (the HSA-producing strain derived from the NBRC10746+PEK dprb1 strain); and 4: a strain in which the prb1 gene has been interrupted, into which a chaperone (OmPDI1/OmERO1/OmKar2) has been introduced, and in which the aox1 gene has been disrupted (the HSA-producing strain derived from the Δaox1+PEK dprb1 strain).

FIG. 9 shows the secretory production amount of the HSA protein induced by methanol (3 L Jar scale) [Jar1: the NBRC 10746 strain into which a chaperone (OmPDI1/OmERO1/OmKar2) has been introduced (the HSA-producing strain derived from the NBRC10746+PEK strain); Jar2: the NBRC 10746 strain into which a chaperone (OmPDI1/OmERO1/OmKar2) has been introduced (the HSA-producing strain derived from the NBRC10746+PEK strain) (nitrogen source fed-batch culture; pH 7 control); Jar3: a strain in which the prb1 gene has been interrupted, into which a chaperone (OmPDI1/OmERO1/OmKar2) has been introduced, and in which the aox1 gene has been disrupted (the HSA-producing strain derived from the Δaox1+PEK dprb1 strain) (nitrogen source fed-batch culture; pH 7 control).

FIG. 10 shows the secretory production amount of the HSA protein achieved by carbon source starvation-induced culture (3 L Jar scale) [Jar1: a strain in which the prb1 gene has been interrupted, into which a chaperone (OmPDI1/OmERO1/OmKar2) has been introduced, and in which the aox1 gene has been disrupted (the HSA-producing strain derived from the Δaox1+PEK dprb1 strain) (low methanol-induced culture); Jar2: a strain in which the prb1 gene has been interrupted, into which a chaperone (OmPDI1/OmERO1/OmKar2) has been introduced, and in which the aox1 gene has been disrupted (the HSA-producing strain derived from the Δaox1+PEK dprb1 strain) (carbon source starvation-induced culture).

### EMBODIMENTS FOR CARRYING OUT THE INVENTION

### 1. Transformed Yeast

The transformed yeast of the present invention is obtained by introduction of a chaperone gene, disruption of the aox1 gene, and/or disruption of the protease gene. Specifically, a transformed yeast into which a chaperone gene has been introduced and in which the aox1 gene has been disrupted, a transformed yeast into which a chaperone gene has been introduced and in which the protease gene has been disrupted, and a transformed yeast into which a chaperone gene has been introduced and in which the aox1 gene and a protease gene have been disrupted are within the scope of the transformed yeast of the present invention.

### (Host Cells)

Host cells to be transformed are preferably yeast strains. Examples of yeast strains include methhylotrophic yeast strains such as Ogataea minuta, Pichia lindneri, Pichia pastoris, Hansenulla polymorpha (Pichia angusta), and Candida boidinii and yeast strains such as Saccharomyces cerevisiae, Kluyveromyces lactis, Yarowia lipolytica, and Shizosaccharomyces pombe, with methhylotrophic yeast strains being preferable. A specific example of the Ogataea minuta strain is the Ogataea minuta YK3 strain (Δoch1Δpep4Δprb1Δyps1Δura3Δade1), and a specific example of the Saccharomyces cerevisiae strain is the Saccharomyces cerevisiae BY4741 strain (MATa Δhis3Δleu2Δmet15Δura3), although yeast strains are not limited thereto.

### (Introduction of Chaperone Gene)

Examples of chaperone genes used in the present invention include genes encoding PDI1 (SEQ ID NO: 35 (the base sequence); SEQ ID NO: 36 (the amino acid sequence)), ERO1 (SEQ ID NO: 43 (the base sequence); SEQ ID NO: 44 (the amino acid sequence)), Kar2 (SEQ ID NO: 47 (the base sequence); SEQ ID NO: 48 (the amino acid sequence)), MPD1 (SEQ ID NO: 37 (the base sequence); SEQ ID NO: 38 (the amino acid sequence)), SCJ1 (SEQ ID NO: 39 (the base sequence); SEQ ID NO: 40 (the amino acid sequence)), EUG1 (SEQ ID NO: 41 (the base sequence); SEQ ID NO: 42 (the amino acid sequence)), and HSP104 (SEQ ID NO: 45 (the base sequence); SEQ ID NO: 46 (the amino acid sequence)) derived from Ogataea minuta (O. minuta).

The chaperone gene used in the present invention may be a chaperone gene derived from another organism species, such as other types of yeast, mold, or a human.

As a chaperone gene derived from another type of yeast, for example, a chaperone gene derived from Saccharomyces cerevisiae can be used. Specific examples include genes encoding PDI1 (Primary SGDID: 5000000548; SEQ ID NO: 49 (the base sequence); SEQ ID NO: 50 (the amino acid sequence)), MPD1 (Primary SGDID: 5000005814; SEQ ID NO: 51 (the base sequence); SEQ ID NO: 52 (the amino acid sequence)), SCJ1 (Primary SGDID: S000004827; SEQ ID NO: 53 (the base sequence); SEQ ID NO: 54 (the amino acid sequence)), ERO1 (Primary SGDID: 5000004599; SEQ ID NO: 55 (the base sequence); SEQ ID NO: 56 (the amino acid sequence)), FKB2 (Primary SGDID: 5000002927; SEQ ID NO: 57 (the base sequence); SEQ ID NO: 58 (the amino acid sequence)), JEM1 (Primary SGDID: S000003609; SEQ ID NO: 59 (the base sequence); SEQ ID NO: 60 (the amino acid sequence)), LHS1 (Primary SGDID: 5000001556; SEQ ID NO: 61 (the base sequence); SEQ ID NO: 62 (the amino acid sequence)), MPD2 (Primary SGDID: 5000005448; SEQ ID NO: 63 (the base sequence); SEQ ID NO: 64 (the amino acid sequence)), ERJ5 (Primary SGDID: S000001937; SEQ ID NO: 65 (the base sequence); SEQ ID NO: 66 (the amino acid sequence)), and EUG1 (Primary SGDID: 5000002926; SEQ ID NO: 67 (the base sequence); SEQ ID NO: 68 (the amino acid sequence)). Sequence information regarding genes derived from Saccharomyces cerevisiae is available from SGD (Saccharomyces genome database: http://www.yeastgenome.org/).

Examples of chaperone genes derived from a human include genes encoding PDI (GenBank Accession No. BC010859; SEQ ID NO: 69 (the base sequence); SEQ ID NO: 70 (the amino acid sequence)), ERO1-Lα (GenBank Accession No. AF081886; SEQ ID NO: 71 (the base sequence); SEQ ID NO: 72 (the amino acid sequence)), ERO1-Lβ (GenBank Accession No. BC044573; SEQ ID NO: 73 (the base sequence); SEQ ID NO: 74 (the amino acid sequence)), and GRP78 (GenBank Accession No. AL354710; SEQ ID NO: 75 (the base sequence); SEQ ID NO: 76 (the amino acid sequence)).

The chaperone gene used in the present invention may be a gene encoding a protein that consists of an amino acid sequence derived from any of the amino acid sequences described above by deletion, substitution, and/or addition of one or several amino acids, provided that such gene has activity of promoting foreign protein secretion. The number of amino acids that may be deleted, substituted, and/or added is preferably 1 to several. The number represented by the term “several” is not particularly limited. For example, such number may be 50 or less, 40 or less, 30 or less, 25 or less, 20 or less, 15 or less, 12 or less, 10 or less, 9 or less, 8 or less, 7 or less, 6 or less, 5 or less, 4 or less, 3 or less, or 2. The term “mutation” used herein primarily refers to a mutation that is artificially introduced via a known method for preparing a mutant protein, and the term may refer to a mutation that is similar to one existing in nature. The term “foreign protein” is used in the same sense as the term “target protein” herein.

Also, the chaperone gene used in the present invention may be a gene encoding a protein that consists of an amino acid sequence having at least 80% sequence identity with any of the amino acid sequences described above and has activity of promoting foreign protein secretion. Specific examples include a gene that consists of a base sequence having at least 80% sequence identity with the base sequence as shown in SEQ ID NO: 35, 37, 39, 41, 43, 45, 47, 49, 51, 53, 55, 57, 59, 61, 63, 65, 67, 69, 71, 73, or 75 and encodes a protein having activity of promoting foreign protein secretion; and a gene encoding a protein that consists of an amino acid sequence having at least 80% sequence identity with the amino acid sequence as shown in SEQ ID NO: 36, 38, 40, 42, 44, 46, 48, 50, 52, 54, 56, 58, 60, 62, 64, 66, 68, 70, 72, 74, or 76 and has activity of promoting foreign protein secretion. The term “at least 80% sequence identity” preferably refers to at least 85% sequence identity, more preferably at least 90% sequence identity, further preferably at least 95%, and most preferably at least 99% sequence identity. A protein homology search can be carried out with the use of, for example, the DNA Databank of Japan (DDBJ) via FASTA, BLAST, or another program.

The chaperone gene used in the present invention may be a gene that hybridizes under stringent conditions to DNA consisting of a base sequence complementary to DNA consisting of any of the base sequences described above and encodes a protein having activity of promoting foreign protein secretion. Under the aforementioned “stringent conditions,” a so-called specific hybrid is formed, but a non-specific hybrid is not formed. Under such conditions, for example, complementary strands of a nucleic acid exhibiting a high degree of sequence identity, i.e., a nucleic acid consisting of a base sequence having at least 80%, preferably at least 85%, more preferably at least 90%, further preferably at least 95%, and most preferably at least 99% sequence identity with any of the base sequences above, undergo hybridization, but complementary strands of a nucleic acid having lesser degrees of sequence identity do not undergo hybridization. More specifically, the sodium salt concentration is 15 to 750 mM, preferably 50 to 750 mM, and more preferably 300 to 750 mM, the temperature is 25° C. to 70° C., preferably 50° C. to 70° C., and more preferably 55° C. to 65° C., and the formamide concentration is 0% to 50%, preferably 20% to 50%, and more preferably 35% to 45%. Under stringent conditions, further, a filter is generally washed at a sodium salt concentration of 15 to 600 mM, preferably 50 to 600 mM, and more preferably 300 to 600 mM, and a temperature of 50° C. to 70° C., preferably 55° C. to 70° C., and more preferably 60° C. to 65° C., after hybridization.

A person skilled in the art can easily obtain such homologous genes by referring to, for example, Molecular Cloning (Sambrook, J. et al., Molecular Cloning: A Laboratory Manual 2nd ed., Cold Spring Harbor Laboratory Press, 10 Skyline Drive Plainview, N.Y., 1989). Also, a base sequence identity search can be carried out via FASTA, BLAST, or other programs.

The amino acid mutation mentioned above, such as deletion, substitution, and/or addition, can be introduced via a technique known in the art, such as the Kunkel method or the Gapped duplex method, or via a technique in accordance therewith. For example, mutagenesis kits utilizing site-directed mutagenesis, such as a Mutant-K (Takara Bio Inc.), Mutant-G (Takara Bio Inc.), or LA PCR in vitro Mutagenesis series kit (Takara Bio Inc.), can be used.

Also, chaperone genes derived from other organism species may be codon-modified genes that are modified so as to improve translation efficiency via substitution of a base sequence with a codon that is frequently used in a host cell. A specific example is a codon-modified gene of a gene encoding PDI derived from a human. DNA having a modified base sequence can be artificially synthesized. In the case of a long DNA sequence, the sequence is first divided into several fragments, fragments are synthesized in advance, and the resultants are then bound to each other at the end.

In the present invention, one or more types of the aforementioned chaperone genes are used in combination. When two or more genes are used in combination, such genes may be derived from the same or different organism species.

Preferable examples of the chaperone genes that are used in the present invention include the pdi1 gene derived from O. minuta, the ero1 gene derived from O. minuta, the kar2 gene derived from O. minuta, the pdi1 gene derived from S. cerevisiae, the pdi gene derived from a human, the ero1 gene derived from a human, and a gene encoding a protein that consists of an amino acid sequence having at least 80%, preferably at least 85%, more preferably at least 90%, further preferably at least 95%, and most preferably at least 99% sequence identity with any of the amino acid sequences above and has activity of promoting foreign protein secretion.

Further preferable examples of the chaperone genes that are used in the present invention include a combination of the pdi1 gene, the ero1 gene, and the kar2 gene derived from O. minuta, a combination of the pdi1 gene and the ero1 gene derived from O. minuta, a combination of the pdi1 gene and the kar2 gene derived from O. minuta, a combination of the pdi gene derived from a human and the ero1 gene derived from O. minuta, a combination of the pdi gene, the ero1-Lβ gene, and the grp78 gene derived from a human, a combination of the pdi gene derived from a human, the ero1 gene derived from O. minuta, and the grp78 gene derived from a human, and a combination of genes each encoding a protein that consists of an amino acid sequence having at least 80%, preferably at least 85%, more preferably at least 90%, further preferably at least 95%, and most preferably at least 99% sequence identity with any of the amino acid sequences of the combinations of the chaperones described above and has activity of promoting foreign protein secretion.

The most preferable examples of the chaperone genes that are used in the present invention include a combination of the pdi1 gene, the ero1 gene, and the kar2 gene derived from O. minuta, or a combination of genes each encoding a protein that consists of an amino acid sequence having at least 80% sequence identity with any of the amino acid sequences of PDI1, ERO1, and Kar2 derived from O. minuta and has activity of promoting foreign protein secretion (e.g., a combination of genes each that consists of the base sequence having at least 80%, preferably at least 85%, more preferably at least 90%, further preferably at least 95%, and most preferably at least 99% sequence identity with the base sequence as shown in SEQ ID NO: 35, 43, or 47 and encodes a protein having activity of promoting foreign protein secretion; or a combination of genes each encoding a protein that consists of an amino acid sequence having at least 80%, preferably at least 85%, more preferably at least 90%, further preferably at least 95%, and most preferably at least 99% sequence identity with the amino acid sequence as shown in SEQ ID NO: 36, 44, or 48 and has activity of promoting foreign protein secretion). Regarding the gene-related notation used herein, for example, the term “a gene encoding PDI1” is used in the same sense as the term “the pdi1 gene.”

The chaperone gene is introduced into yeast, which is a host cell, with the use of an expression vector. In the present invention, an expression vector can be introduced into a host cell by any method, provided that the introduced gene is stably present and adequately expressed in a yeast host. Examples of methods that are generally employed include the calcium phosphate method (Ito et al., Agric. Biol. Chem., 48, 341, 1984), electroporation (Becker, D. M. et al., 1990; Methods. Enzymol., 194, 182-187), use of spheroplasts (Creggh et al., Mol. Cell. Biol., 5, 3376, 1985), the lithium acetate method (Itoh, H., 1983; J. Bacteriol. 153, 163-168), and lipofection.

(Disruption of Aox1 Gene and/or Disruption of Protease Gene)

The transformed yeast of the present invention is obtained by, in addition to the introduction of a chaperone gene, disruption of the aox1 gene endogenous in the host genome, disruption of the protease gene endogenous in the host genome, or disruption of both the aox1 gene and the protease gene endogenous in the host genome.

Examples of the aox1 genes include a gene encoding AOX1 derived from O. minuta (SEQ ID NO: 27 (the base sequence); SEQ ID NO: 28 (the amino acid sequence)), a gene encoding AOX1 derived from Pichia pastoris (GenBank accession number: U96967), and a gene encoding AOX1 derived from Candida boidinii (GenBank accession number: Q00922). The aox1 gene is not limited thereto, provided that the gene encodes AOX1 derived from yeast.

Examples of the protease genes include the prb1 gene (SEQ ID NO: 31 (the base sequence); SEQ ID NO: 32 (the amino acid sequence)) and the pep4 gene (GenBank accession number: AB236164) derived from O. minuta, the prb1 gene (GenBank accession number: AB060541) and the pep4 gene (JP 2000-078978 A) derived from Candida boidinii, the prb1 gene and the pep4 gene derived from Pichia pastoris, the prb1 gene (GenBank accession number: M18097) derived from Saccharomyces cerevisiae, and the prb1 gene (GenBank accession number: A75534) derived from Kluyveromyces lactis. The protease gene is not limited thereto, provided that the gene is derived from yeast.

Accordingly, the transformed yeast of the present invention is preferably obtained by, in addition to the introduction of a chaperone gene, disruption of the aox1 gene endogenous in the host genome or disruption of the prb1 gene endogenous in the host genome. More preferably, the transformed yeast is obtained by, in addition to the introduction of a chaperone gene, disruption of both the aox1 gene and the prb1 gene endogenous in the host genome. It is most preferable that, in addition to the introduction of, as the chaperon gene, a combination of the pdi1 gene, the ero1 gene, and the kar2 gene derived from O. minuta or a combination of genes each encoding a protein that consists of an amino acid sequence having at least 80%, preferably at least 85%, more preferably at least 90%, further preferably at least 95%, and most preferably at least 99% sequence identity with any of the amino acid sequences of PDI1, ERO1, and Kar2 and has activity of promoting foreign protein secretion, both the aox1 gene and the prb1 gene endogenous in the host genome are disrupted.

In the present invention, the term “gene disruption” refers to “gene deletion” whereby all or a part of the target gene is deleted from the chromosome, substitution of the target gene, and “gene interruption” that inhibits the expression of a functional protein encoded by a target gene by interruption of the target gene without deleting such gene. From the viewpoint of disruption of functions of the target gene, gene disruption may take the form of mutagenesis or expression inhibition of a gene causing functional deficiency. A means for gene disruption is not particularly limited, provided that the expression or functions of a protein encoded by the target gene is/are inhibited or deleted.

Typically, the target gene can be disrupted via homologous recombination. At the outset, the target gene is interrupted or partially deleted, an adequate selection marker gene is inserted thereinto, and a DNA construct comprising a selection marker flanked by the upstream region and the downstream region of the target gene is prepared. Subsequently, this construct is introduced into a yeast strain, so as to perform recombination in homologous regions at both ends of the introduced fragment (a DNA construct comprising a selection marker) and the target gene in the chromosome, and the target gene in the chromosome is then substituted with the introduced fragment. In such a case, a selection marker used for gene disruption can be an auxotrophic marker or a drug-tolerant marker, as described below.

An embodiment involving the use of the ura3 gene as a selection marker is specifically described. A plasmid comprising the ura3 gene having repeat structures before and after the structural gene is constructed, the gene cassette is cleaved with a restriction enzyme, and the resultant is inserted into the target gene of a plasmid, so as to construct the disrupted alleles. This plasmid is substituted with the target gene of the chromosome, so as to obtain a gene-disrupted strain. The ura3 gene inserted into the chromosome has repeat structures before and after the ura3 gene, homologous recombination takes place between repeat sequences, and the ura3 gene is thus deleted from the chromosome. The deleted strain can be selected with the use of 5-fluoroorotic acid (5-FOA). The ura3 variant is resistant to 5-FOA (Boeke et al., Mol. Gen. Genet., 197, 345-346, 1984; Boeke et al., Methods Enzymol., 154, 165-174, 1987), and strains having URA+ phenotypes cannot grow in a 5-FOA medium. If a strain exhibiting tolerance is separated with the use of a medium supplemented with 5-FOA, accordingly, the use of the ura3 gene marker becomes possible again. In general, use of a selection marker is necessary in order to disrupt a gene. With the use of the ura3 gene, however, ura3 traits can be efficiently reproduced.

A mutation aimed at causing functional defects can be introduced into a gene by modifying a gene via mutagenesis, such as site-directed mutagenesis. Specifically, a gene mutation aimed at causing functional defects at a particular site is, for example, a mutation that has been caused by frame-shift or amino acid substitution at the active center resulting from insertion or deletion of nucleotides into or from ORF, and the gene is mutated to encode a protein that has been inactivated. When gene expression is suppressed, the expression level of the relevant gene is lowered or lost. Examples of methods for suppressing gene expression include a method involving the use of antisense RNA or RNAi and a method comprising attenuating a promoter.

### (Expression Vector)

The chaperone gene is introduced into yeast with the use of an expression vector. Examples of such expression vector include a vector comprising a single type of chaperone gene, a vector comprising two or more copies of a single type of chaperone gene, and a vector comprising a combination of two or more types of chaperone genes. In order to express the chaperone gene in yeast, a vector comprising a single gene may be used to carry out transformation. Alternatively, a vector comprising a plurality of genes may be used to carry out transformation. Also, such expression vector may comprise a gene encoding a foreign protein. Alternatively, aiming high expression and secretion, expression vectors comprising a gene encoding a foreign protein may be prepared separately. In such a case, vectors are cotransfected into a host cell.

A gene encoding a foreign protein is not particularly limited. Examples include: various enzyme genes, such as the lysozyme gene, the α-amylase gene, and the α-galactosidase gene, and in particular, glycosyltransferase genes that are necessary for production of pharmaceutically useful glycoproteins, such as the erythropoietin (EPO) gene and granulocyte-colony stimulating factor (G-CSF) genes; various interferon genes that are pharmaceutically useful and physiologically active proteins, such as interferon α and interferon γ genes; various interleukin genes, such as IL1 and IL2 genes; various cytokine genes, such as the erythropoietin (EPO) gene and the granulocyte-colony stimulating factor (G-CSF) gene; growth factor genes; and various vaccine antigens such as influenza. Such genes may be obtained via any means.

The present invention is particularly effective on a protein that is highly hydrophobic and a protein whose secretory production is insufficient due to composite formation. Thus, the aforementioned foreign protein includes a multimeric protein, such as an antibody or a functional fragment thereof; i.e., a heteromultimer.

An expression regulation region may be adequately added to the chaperone gene or a gene encoding a foreign protein to constitute an expression vector as a protein expression unit. A protein expression unit comprises, in the direction of a transcription reading frame, at least a promoter region, the above gene, and a transcription terminator region. A promoter that can be used herein may be an inducible expression promoter or constitutive expression promoter. Examples of inducible expression promoters include a promoter of a gene encoding alcohol oxidase (AOX), a promoter of a gene encoding dihydroxyacetone synthase (DAS), and a promoter of a gene encoding formate dehydrogenase (FDH) involved in the methanol metabolism of methhylotrophic yeast. An example of another inducible promoter that can be used is a copper-inducible promoter (CUP). Examples of constitutive expression promoters include promoters of the genes encoding glyceraldehyde-3-phosphate dehydrogenase (TDH, GAP), phosphoglycerokinase (PGK), phosphotriose isomerase (TPI), enolase (ENO), actin (ACT), cytochrome c (CYC), trehalose synthase (TPS), and alcohol dehydrogenase (ADH). Also, a transcription terminator may be a sequence having activity of terminating transcription from a promoter. It may have the same or a different sequence as the gene of the promoter.

Also, an expression vector may comprise DNA encoding a secretory signal sequence that functions in a yeast cell added to a gene encoding a foreign protein. Thus, secretory production becomes possible, and a foreign protein of interest can be easily isolated and purified. Examples of secretory signal sequences include a secretory signal sequence of an S. cerevisiae-derived α-mating factor (αMF), a secretory signal sequence of S. cerevisiae-derived invertase (SUC2), and a secretory signal sequence of human-derived α-galactosidase.

The expression vector can comprise a selection marker for selecting a transformant. For example, yeast expression vectors can comprise auxotrophic marker genes selected from among his1, his2, his3, his4, his5, his6, leu2, arg1, arg2, arg3, trp1, lys2, ade1, ade2, ura3, and ura5 genes.

As selection markers, drug-resistant markers that impart resistance to drugs such as cerulenin, aureobasidin, Zeocin, canavanine, cycloheximide, hygromycin, blasticidin, tetracycline, kanamycin, ampicillin, tetracycline, and neomycin can be used, in addition to the aforementioned auxotrophic markers. Thus, transformants can be selected. Also, genes that impart solvent resistance to ethanol, osmotic resistance to glycerol or salt, metal ion resistance to copper, and the like may be used as markers, so that transformants can be selected.

### 2. Method for Producing Protein

In the present invention, proteins can be produced by culturing the transformed yeast obtained in 1. above via a conventional technique and sampling the proteins from the culture product, followed by purification. The term “culture product” used herein refers to culture cells, cultured strains, or disrupted cells or strains, in addition to a culture supernatant.

When the host cell is yeast, either a natural or synthetic medium may be used for culture, provided that it contains carbon sources, nitrogen sources, and inorganic salts assimilable by the yeast and permits efficient culture of the transformed yeast. Examples of carbon sources that can be used include: carbohydrates such as glucose, fructose, sucrose, and starch; organic acids such as acetic acid, lactic acid, citric acid, and propionic acid; and alcohols such as methanol, ethanol, propanol, and glycerol. Examples of nitrogen sources include: ammonia; ammonium salts of inorganic or organic acids such as ammonium chloride, ammonium sulfate, ammonium acetate, ammonium phosphate, and ammonium carbonate; other nitrogen-containing compounds such as urea; nitrogenous organic substances such as amino acids, yeast extracts, peptone, meat extracts, corn steep liquor, casein hydrolysate, soybean cake, and soybean cake hydrolysate. Examples of inorganic salts include: monopotassium phosphate, dipotassium phosphate, magnesium phosphate, magnesium sulfate, sodium chloride, iron(I) sulfate, manganese sulfate, copper sulfate, and calcium carbonate. In the case of an auxotrophic yeast, nutritive substances necessary for the growth thereof may be added to a medium. Examples of such nutritive substances include amino acids, vitamins, nucleic acids, and salts. In accordance with the type of selection marker, an antibiotic agent, such as aureobasidin, ampicillin, or tetracycline, may be adequately added to a medium. Alternatively, an amino acid that can be supplied by a gene complementing auxotrophy (e.g., leu, ura, or trp) may be removed.

When culturing yeast transformed with the use of an expression vector comprising an inducible promoter, an inducer may be added to the medium, according to need. When culturing yeast transformed with the use of an expression vector comprising a methanol-inducible promoter (e.g., aox, das, or mox), for example, methanol is added to the medium. When culturing yeast transformed with the use of an expression vector comprising a GAL promoter, galactose is added to the medium.

During culture, an inhibitor of PMT activity may be added to the medium, so as to inhibit addition of an O-sugar chain peculiar to yeast. Examples of inhibitors of PMT activity include the rhodanine-3-acetic acid derivative (5-[[3,4-(1-phenylmethoxy)phenyl]methylene]-4-oxo-2-thioxo-3-thiazolidineacetic acid, compound (1c) described in Bioorganic & Medicinal Chemistry Letters, Vol. 14, p. 3975, 2004) and {(5Z)-4-oxo-5-[3-(1-phenylethoxy)-4-(2-phenylethoxy)benzylidene]-2-thioxo-1,3-thiazolidin-3-yl}acetic acid (compound (5a) described in Bioorganic & Medicinal Chemistry Letters, Vol. 14, p. 3975, 2004).

Culture is carried out at about 20° C. to 30° C. for 24 to 1,000 hours. Culture can be carried out via batch culture or continuous culture, such as static, shake, agitation, or aeration culture.

When the transformed yeast of the present invention has been transformed with the use of an expression vector comprising a methanol-inducible promoter, it is cultured in a medium supplemented with methanol. Methanol may be added to the medium with the use of a pump or by other means while observing the growth of yeast. Such addition may be continuously or intermittently carried out, and it is not necessary to perform addition over the entire period of culture. When culturing a transformed yeast strain (i.e., a strain in which the aox1 gene has been disrupted), for example, methanol-induced culture is initiated in a medium supplemented with methanol at a concentration of preferably 0.3% to 2% (v/v) and more preferably 0.5% to 1% (v/v). When the methanol concentration is reduced to about 0.2% to 0.3% (v/v), methanol may be intermittently added, preferably at a final concentration of 0.1% to 0.5% (v/v), and more preferably at a final concentration of 0.2% to 0.4% (v/v) per day.

When carbon sources are depleted from the medium used for the growth of the transformed yeast and the medium is brought to a carbon-starved state, a methanol-inducible promoter is strongly activated. After the medium has been depleted of the carbon sources, accordingly, the carbon source content in the medium may be adequately adjusted. Thus, culture can be conducted without the addition of methanol or at a low methanol concentration of 0.2% (v/v) or lower. Examples of “carbon sources” include glycerin, alanine, mannitol, sorbitol, trehalose, and lactose. When “the carbon source content in the medium may be adequately adjusted,” the carbon sources may be added to the medium at the lowest concentration necessary for the growth of the transformed yeast of the present invention and protein expression. Specifically, culture may be initiated under carbon source-depleted conditions (carbon source-starvation conditions) when the target cell density is attained. When culturing a transformed yeast strain (a strain in which the aox gene has been disrupted), for example, whether or not the cell density has reached the target level and carbon sources have been depleted is confirmed. At the same time, glycerin and sorbitol may be continuously added at a concentration of preferably 0.5% to 6% (w/v), and more preferably 1% to 4% (w/v) per day.

It is preferable that culture be carried out under conditions in which protease activity is inhibited. Thus, degradation of the target protein that is secreted and produced by yeast is inhibited, and the secretory production amount significantly increases. Protease activity can be inhibited by disrupting the proteinase B gene of yeast as described above, and it can be inhibited by regulating the pH level of the medium. The pH level of the medium is preferably 6.0 to 7.5, so that activity of acidic protease such as proteinase A in the medium is inhibited and the growth of the yeast is not affected. The pH level is regulated with the use of, for example, inorganic acid, organic acid, or an alkaline solution.

Further, culture is preferably carried out under conditions in which nitrogen sources are continuously added. By supplying nitrogen sources during culture, secretory production of proteins and maintenance thereof are remarkably improved. The final concentration of the nitrogen sources to be added per day is preferably 0.1% to 0.75% (w/v) in the case of a yeast extract and 0.05% to 0.15% (w/v) in the case of L-histidine monohydrochloride monohydrate, and it is more preferably 0.3% to 0.5% (w/v) in the case of a yeast extract and 0.1% to 0.13% (w/v) in the case of L-histidine monohydrochloride monohydrate. Nitrogen sources can be added with the use of a mixture of a yeast extract and L-histidine monohydrochloride monohydrate.

A transformed yeast into which a chaperone gene has been introduced and in which the aox1 gene and the protease gene have been disrupted, which is an embodiment of the transformed yeast of the present invention, is cultured in a medium with the pH level within the aforementioned range supplemented with nitrogen sources. Thus, the amount of methanol to be added can be reduced to 4% to 7% of the amount of methanol added, compared with the case of a transformed yeast into which only the chaperone gene has been introduced.

The expression product of a gene of a foreign protein from the culture product (i.e., a culture solution or culture cells) can be identified via SDS-PAGE, Western blotting, ELISA, or the like. The produced proteins may be isolated and purified via conventional techniques for protein isolation and purification. When target proteins are produced in the cells after culture, the cells may be pulverized using, for example, an ultrasonic pulverizer, a French press, a Manton-Gaulin homogenizer, or a Dyno-mil, to obtain target proteins. When the target proteins are produced outside the cells, the culture solution is used as it is, or the cells are removed via centrifugation or the like. Thereafter, the target proteins are collected via extraction using an organic solvent, subjected to various chromatography techniques (e.g., hydrophobic, reversed-phase, affinity, or ion-exchange chromatography), gel filtration using molecular sieves, electrophoresis using polyacrylamide gel, or the like, according to need. These techniques may be employed solely or in combinations of two or more.

The above culture and purification techniques are examples, and methods are not limited thereto. The amino acid sequence of the purified gene product can be confirmed by a conventional amino acid analysis method, such as automated amino acid sequencing via the Edman degradation technique.

### EXAMPLES

Hereafter, the present invention is described in detail with reference to the examples, although the technical scope of the present invention is not limited to the examples. Plasmids, restriction enzymes, DNA modifying enzymes, and the like that are used in the examples of the present invention are commercially available products, and these products can be used in accordance with conventional techniques. Also, procedures of DNA cloning, nucleotide sequencing, yeast transformation, culture of transformed yeast, and the like are well-known in the art or can be learned through existing publications.

### [Example 1] Construction of Vector for Foreign Gene Expression

(1) Construction of Vector for Foreign Gene Introduction Carrying a Zeocin-Resistant Gene as a Selection Marker and Comprising the Aox1 Gene Promoter of NBRC 10746 (O. minuta, Biological Resource Center, NITE) and the Terminator Cassette

NBRC 10746 AOX1 (GenBank Accession Number AB242209) comprises an amino acid sequence of 663 amino acids encoded by a 1,992-bp base sequence (SEQ ID NO: 27, SEQ ID NO: 28). PCR was carried out using the genomic DNA of NBRC 10746 prepared with the use of the Y-DER Yeast DNA Extraction Reagent (78870, PIERCE) as a template, the Hd AOXp Fw primer (5′-GCAAGCTTTCTTTCGCAAACAGCTCTTTG-3′: SEQ ID NO: 1), the AOXp ry primer (5′-GAACCCGGGAACAGAATCTAGATTTTTTCGTAAGTCGTAAG-3′: SEQ ID NO: 2), and the PrimeSTAR Max DNA Polymerase (RO45A, Takara Bio Inc.) at 98° C. for 10 seconds, 55° C. for 5 seconds, and 72° C. for 15 seconds, and this cycle was repeated 30 times. Thus, an aox1 promoter region-containing DNA fragment comprising the aox1 promoter region of about 2.4 kbp and a spacer region of 22 bp was amplified. Also, PCR was carried out using DNA of NBRC 10746 as a template, the AOXt fw primer (5′-CTGTTCCCGGGTTCCTGGATCCGAGACGGTGCCCGACTC-3′: SEQ ID NO: 3), and the Kp AOXt Rv primer (5′-GCGGTACCGTTAGTGGTACGGGCAG-3′: SEQ ID NO: 4) at 98° C. for 10 seconds, 55° C. for 5 seconds, and 72° C. for 5 seconds, and this cycle was repeated 30 times. Thus, an aox1 terminator region-containing DNA fragment comprising the aox1 terminator region of about 0.8 kbp and a spacer region of 22 bp was amplified. PCR was carried out using these DNA fragments as templates, the Hd AOXp Fw primer, and the Kp AOXt Rv primer at 98° C. for 10 seconds, 55° C. for 5 seconds, and 72° C. for 15 seconds, and this cycle was repeated 30 times. Thus, the aox1 promoter region of about 2.4 kbp was ligated to the terminator region of about 0.8 kbp, and the resultant was then amplified. The ligated DNA fragment was subjected to agarose electrophoresis, recovered, and then cloned into pCR-Blunt II-TOPO. The cloned plasmid was subjected to double digestion with the restriction enzymes HindIII and KpnI to obtain a DNA fragment comprising the aox1 gene promoter and the terminator cassette.

The pOMexGP1Z plasmid described in WO 2009/057813 (i.e., a vector for foreign gene expression carrying a Zeocin-resistant gene as a selection marker and comprising the gap gene promoter and the terminator cassette) was subjected to double digestion with the restriction enzymes HindIII and KpnI to obtain a DNA fragment comprising a Zeocin-resistant gene marker. The DNA fragment comprising the aox1 gene promoter and the terminator cassette was introduced into the fragment obtained. Thus, the pOMEA-Z1 plasmid was obtained.

### [Example 2] Preparation of Strain in which the Ura3 Gene has been Disrupted

### (1) Preparation of DNA Fragment for Ura3 Gene Disruption

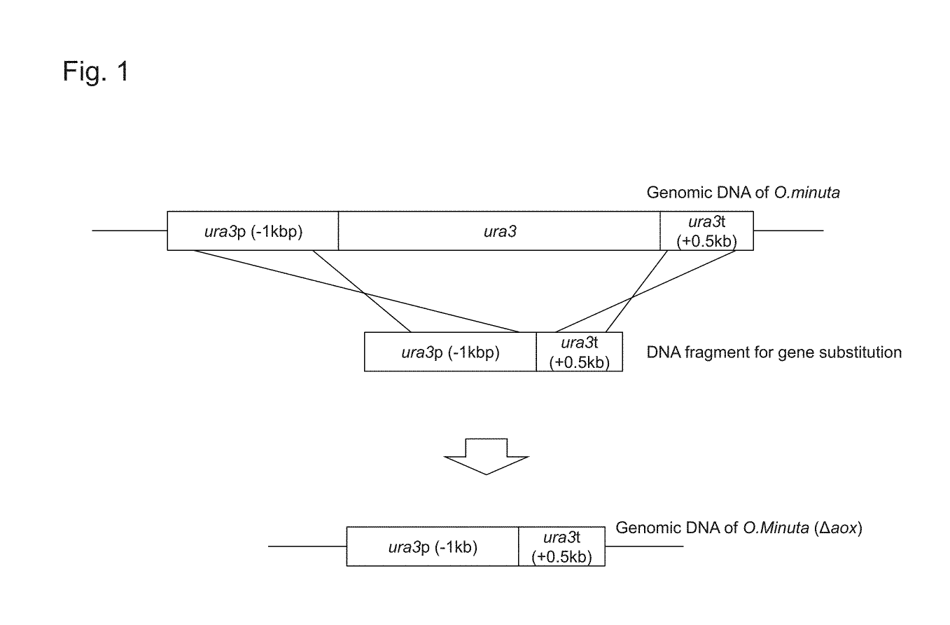

FIG. 1 shows forms of gene disruption using a DNA fragment comprising the ura3 ORF promoter and the terminator. NBRC 10746 URA3 (GenBank Accession Number AB242207) comprises an amino acid sequence of 265 amino acids encoded by a 798-bp base sequence (SEQ ID NO: 29, SEQ ID NO: 30). PCR was carried out using the genomic DNA of NBRC 10746 prepared with the use of the Y-DER Yeast DNA Extraction Reagent (78870, PIERCE) as a template, the dURA Fw primer (5′-GGTACCAGTACTGGAAA-3′: SEQ ID NO: 5), the dURA ry primer (5′-CAGATAAACAGGCGACT TTTCGGGTCACGTGACT-3′: SEQ ID NO: 6), and the PrimeSTAR Max DNA Polymerase (RO45A, Takara Bio Inc.) at 98° C. for 10 seconds, 55° C. for 5 seconds, and 72° C. for 5 seconds, and this cycle was repeated 30 times. Thus, a ura3 terminator region-containing DNA fragment comprising the ura3 terminator region of about 0.5 kbp and a ura3 promoter region of 17 bp was amplified. Also, PCR was carried out using DNA of NBRC 10746 as a template, the dURA fw primer (5′-AGTCACGTGACCCGAAA AGTCGCCTGTTTATCTG-3′: SEQ ID NO: 7), and the dURA Rv primer (5′-CCAAGGAGGAAGAAATT-3′: SEQ ID NO: 8) at 98° C. for 10 seconds, 55° C. for 5 seconds, and 72° C. for 5 seconds, and this cycle was repeated 30 times. Thus, a ura3 promotor region-containing DNA fragment comprising the ura3 promoter region of about 1.2 kbp and a ura3 terminator region of 17 bp was amplified. PCR was carried out using these DNA fragments as templates, the dURA Fw primer, and the dURA Rv primer at 98° C. for 10 seconds, 55° C. for 5 seconds, and 72° C. for 5 seconds, and this cycle was repeated 30 times. Thus, the ura3 promoter region of about 1.2 kbp was ligated to the terminator region of about 0.5 kbp, and the resultant was then amplified. The ligated DNA fragment was subjected to agarose electrophoresis, recovered, and then designated as a fragment for ura3 gene disruption.

(2) Preparation of a Strain in which the Ura3 Gene has been Disrupted

A fragment for ura3 gene disruption was introduced into the NBRC 10746 strain via electroporation. The resultant was inoculated into 5 ml of YPD medium and cultured at 28° C. for 12 to 14 hours up to the logarithmic growth phase (OD₆₀₀=about 0.5 to 4). The strains were recovered via centrifugation at 1400×g for 5 minutes and washed once with 10 ml of ice-cooled sterile water and then washed once with 4 ml of ice-cooled sterile water. The strains were suspended in 2 ml of LC buffer (100 mM LiCl, 50 mM potassium phosphate buffer, pH 7.5), the suspension was shaken at 28° C. for 45 minutes, 0.05 ml of 1 M DTT was added thereto, and the resultant was shaken for an additional 15 minutes. The strains were recovered via centrifugation at 1400×g for 5 minutes, the recovered strains were washed with 8 ml of ice-cooled STM buffer (270 mM sucrose, 10 mM Tris-HCl buffer, pH 7.5, 1 mM MgCl₂) and then with 1 ml of STM buffer, and the resultants were suspended in 0.05 ml of ice-cooled STM buffer. Transformation experiment via an electric pulse method was carried out using the Electro Cell Manipulator ECM 600 (BTX). After 0.05 ml of the cell suspension was mixed with 0.005 ml (3 μg) of a DNA sample of a fragment for ura3 gene disruption, the mixture was introduced into a 0.2-cm disposable cuvette, and electric pulses were applied under adequate conditions (voltage: 1.5 kV, 100-200Ω). Immediately thereafter, YPD medium containing 1 ml of ice-cooled 1 M sorbitol was added, and shake culture was conducted at 28° C. for 4 to 6 hours. After culture, the strains were applied to YPD selection medium containing an adequate amount of antibiotics, and the plate was subjected to culture at 28° C. to obtain transformed colonies. After electroporation, the resultant was applied to a YPAD agar medium (10 g/l yeast extract, 20 g/l peptone, 20 g/l glucose, 40 mg/l adenine-HCl, and 20 g/l agar) containing 5-FOA (5-fluoroorotic acid) at a final concentration of 0.1% (w/v), and the transformants were allowed to proliferate at 28° C. for about 3 days. The proliferated transformants were allowed to proliferate again on YPAD agar medium containing 5-FOA (5-fluoroorotic acid) at a final concentration of 0.1% (w/v). Transformants in which the URA3 gene has been disrupted were selected via colony PCR. Some yeast strains that had proliferated on the YPAD agar medium containing 5-FOA at a final concentration of 0.1% (w/v) were suspended in 10 μl of a 0.25% SDS solution, 90 μl of sterile water was added, and strains were removed via centrifugation at 3,100×g and 4° C. for 5 minutes. The resulting supernatant as a DNA solution was inspected with the use of the dURA check −1.5 kbp primer designed in the upstream sequence of the ura3 promoter (5′-ATCACAGGAAAGCGCAT-3′: SEQ ID NO: 9) and the dURA check+1 kbp primer designed in the downstream sequence of the ura3 terminator (5′-ATTCGAGCATCGCCGTG-3′: SEQ ID NO: 10), and a strain in which a region of about 2.7 kbp without the ura3 coding region has been amplified was designated as the strain in which the ura3 gene has been disrupted (Δura3 strain).

### [Example 3] Preparation of a Strain in which the Aox1 Gene has been Disrupted

### (1) Preparation of Vector for Aox1 Gene Disruption

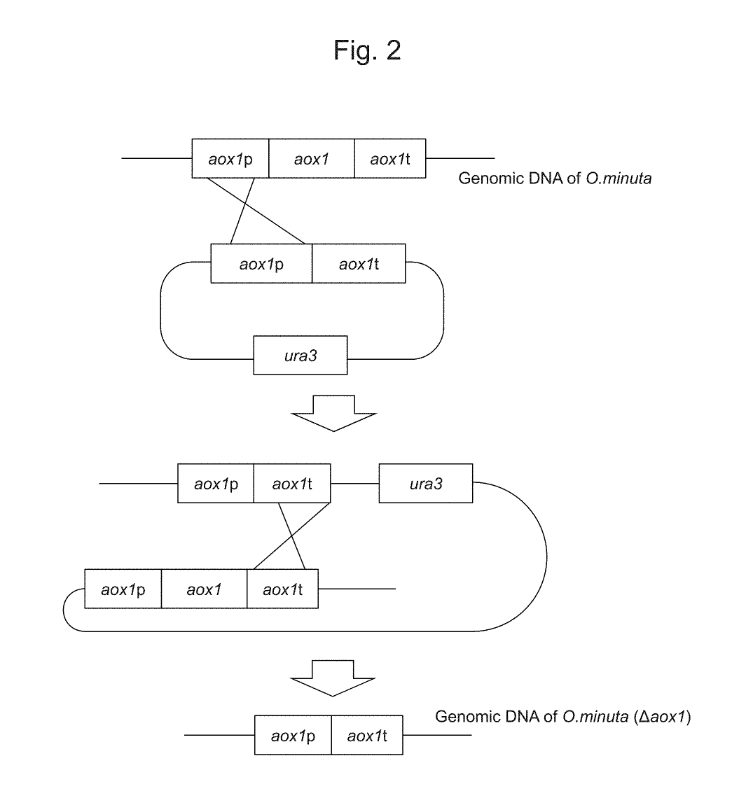

FIG. 2 shows forms of gene disruption using a DNA fragment comprising the aox1 ORF promoter and the terminator region. pOMEA-Z1 constructed in Example 1 was subjected to double digestion with HindIII and KpnI to obtain a DNA fragment comprising the aox1 gene promoter and the terminator cassette. The DNA fragment comprising the aox1 gene promoter and the terminator cassette was introduced into the DNA fragment obtained via double digestion of the onaP09007 plasmid described in WO 2009/057813 (i.e., a constant expression vector for a gene encoding OmKar2) with HindIII and KpnI to obtain the pOMEU1 plasmid.

(2) Preparation of a Strain in which the Aox1 Gene has been Disrupted

The pOMEU1 plasmid was introduced into the strain in which the ura3 gene has been disrupted (Δura3 strain) described in Example 2 via electroporation under the conditions described in Example 2. The pOMEU1 was digested with the restriction enzyme BglII, followed by ethanol precipitation, and the resultant DNA (3 μg) was used. After electroporation, the resultant was applied onto a casamino acid-U-A agar medium (6.7 g/l yeast nitrogen base w/o amino acids, 0.5 g/l casamino acid, 20 g/l glucose, 20 mg/l L-tryptophan, and 20 g/l agar), and it was then allowed to proliferate at 28° C. for about 3 days. The proliferated transformants were allowed to proliferate again on the casamino acid-U-A agar medium to obtain a strain into which the aox1 gene-disrupting plasmid had been introduced.

Subsequently, the strain into which the aox1 gene-disrupting plasmid had been introduced was inoculated into a 50-ml polypropyrene centrifuge tube (227241, Greiner) containing 5 ml of YPAD medium (10 g/l yeast extract, 20 g/l peptone, 20 g/l glucose, and 40 mg/l adenine-HCl), and the upper part of the centrifuge tube was sealed with CO₂ permeable plate seal (676051, Greiner). After the reciprocal shake culture was conducted at an agitation speed of 250 rpm, an amplitude of 25 mm, and a temperature of 28° C. for 2 days, the culture product was applied onto a YPAD medium (1 g/l yeast extract, 2 g/l peptone, 20 g/l glucose, 40 mg/l adenine-HCl, and 20 g/l agar) containing 5-FOA at a final concentration of 0.1% (w/v), and it was then allowed to proliferate at 28° C. for about 3 days. The proliferated transformants were allowed to proliferate again on the YPAD medium containing 5-FOA at a final concentration of 0.1% (w/v), and transformants in which the aox1 gene has been disrupted were selected via colony PCR. Some yeast strains that had proliferated on the YPAD medium containing 5-FOA at a final concentration of 0.1% (w/v) were suspended in 10 μl of a 0.25% SDS solution, 90 μl of sterile water was added thereto, and strains were then removed via centrifugation at 3,100×g and 4° C. for 5 minutes. The resulting supernatant as a DNA solution was inspected with the use of the AOX ORF fw primer (5′-ATGGCTATTCCTGACGAATT-3′: SEQ ID NO: 11) and the AOX ORF ry primer (5′-TTAGAATCTAGCCAGACCCTTC-3′: SEQ ID NO: 12) designed within the aox1 ORF sequence, and a strain in which DNA amplification was not observed was designated as a strain in which the aox1 gene has been disrupted (Δaox1 strain).

### [Example 4] Preparation of Strain into which OmPDI1, OmERO1, and OmKar2 Chaperones have been Introduced

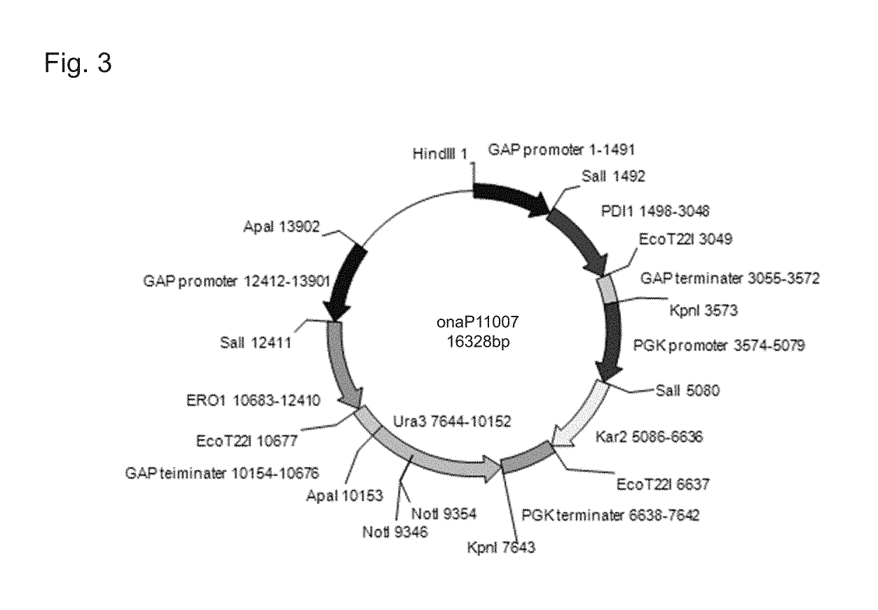

The onaP11007 coexpression vector for OmPDI1, OmERO1, and OmKar2 described in WO 2009/057813 was used (FIG. 3). The onaP11007 plasmid was introduced into the NBRC 10746 strain via electroporation. The onaP11007 plasmid was digested with the restriction enzyme NotI, followed by ethanol precipitation, and the resultant DNA (1 μg) was used. The constructed onaP11007 was digested with the restriction enzyme NotI and then introduced via electroporation described in Example 2. After electroporation, the resultant was applied onto a casamino acid-U-A agar medium (6.7 g/l yeast nitrogen base w/o amino acids, 0.5 g/l casamino acid, 20 g/l glucose, 20 mg/l L-tryptophan, and 20 g/l agar), and it was then allowed to proliferate at 28° C. for about 3 days. The proliferated transformants were allowed to proliferate again on the casamino acid-U-A agar medium, and transformants into which a chaperone has been introduced were selected via colony PCR. Some yeast strains that had proliferated on the casamino acid-U-A agar medium were suspended in 10 μl of a 0.25% SDS solution, 90 μl of sterile water was added thereto, and strains were then removed via centrifugation at 3,100×g and 4° C. for 5 minutes. The resulting supernatant as a DNA solution was inspected in the manner described below. That is, introduction of the pdi1 gene was confirmed by detecting amplification of a DNA fragment of about 1.6 kbp with the use of the GAPpforS-F primer (5′-GATCTCAGGCCGAGTCAAGAC-3′: SEQ ID NO: 13) and the OmPDI-END Rv primer (5′-TTACAACTCGTCGTGAGCC-3′: SEQ ID NO: 14) designed within the gap promoter sequence. Introduction of the ero1 gene was confirmed by detecting amplification of a DNA fragment of about 1.6 kbp with the use of the GAPpforS-F primer (5′-GATCTCAGGCCGAGTCAAGAC-3′: SEQ ID NO: 13) and the OmERO-END Rv primer (5′-TTATAGCTCCAAACGATACAG-3′: SEQ ID NO: 15) designed within the gap promoter sequence. Introduction of the kar2 gene was confirmed by detecting amplification of a DNA fragment of about 2 kbp with the use of the PGKp-END Fw primer (5′-TAAACACTAACGCCGCAT-3′: SEQ ID NO: 16) and the OmKar-END Rv primer (5′-TCACAGCTCATCATGATCC-3′: SEQ ID NO: 17) designed within the pgk promoter sequence. PCR was carried out using TaKaRa LA Taq™ with GC Buffer (RR02AG, TaKaRa Bio) to amplify a target fragment (a cycle of 94° C. for 30 seconds, 55° C. for 30 seconds, and 72° C. for 120 seconds was repeated 30 times). A transformant in which amplification of interest was observed was designated as a strain into which a chaperone gene had been introduced (ura3: pdi1/ero1/kar2 strain, NBRC10746+PEK strain).

### [Example 5] Preparation of a Strain in which the Prb1 Gene has been Interrupted

### (1) Preparation of a Vector for Interrupting the Prb1 Gene

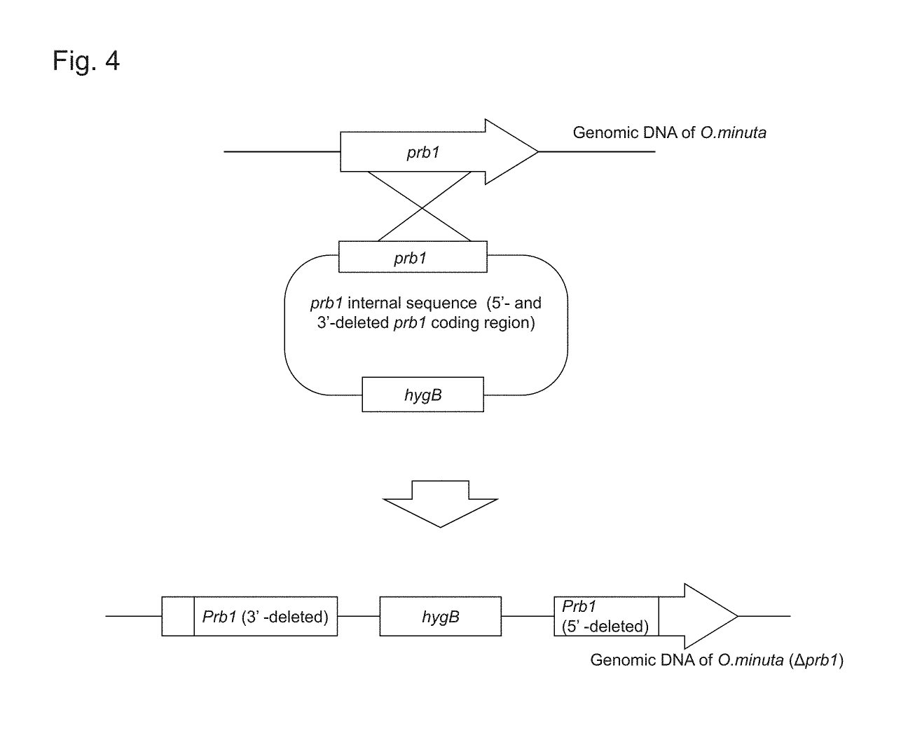

FIG. 4 shows forms of gene disruption using an internal sequence of prb1 ORF. NBRC 10746 PRB1 comprises an amino acid sequence of 539 amino acids encoded by a 1,620-bp base sequence (SEQ ID NO: 31, SEQ ID NO: 32). The pOMexPGHy plasmid described in WO 2009/057813 (a vector for foreign gene expression carrying a hygromycin B-resistant gene as a selection marker and comprising a phosphoglycerin kinase (PGK1) promoter and a terminator) was subjected to double digestion with the restriction enzymes HindIII and KpnI to obtain a DNA fragment comprising the hygromycin-resistant gene marker. PCR was carried out using the genomic DNA of NBRC 10746 prepared with the use of the Y-DER Yeast DNA Extraction Reagent (78870, PIERCE) as a template, the Doprb1F primer (5′-CAAGCTTCGTTGGCAGCAGTGGAG-3′: SEQ ID NO: 18) and the Doprb1R primer (5′-CGGTACCCGATGGAATCTCAGACA-3′: SEQ ID NO: 19) designed within PRB1 ORF, and PrimeStarmax Polymerase (12344-024, TaKaRa Bio) at 98° C. for 10 seconds, 55° C. for 5 seconds, and 72° C. for 5 seconds, and this cycle was repeated 30 times. Thus, a target DNA fragment of about 1.4 kbp as shown in SEQ ID NO: 20 was amplified and then cloned into pCR2.1-TOPO. The base sequence of the inserted DNA fragment was inspected, and it was confirmed to have a prb1 internal sequence. A DNA fragment comprising the prb1 internal sequence was recovered via HindIII-KpnI digestion from the plasmid carrying the DNA fragment in which the base sequence had been inspected with the use of the restriction enzyme HindIII site that had been introduced into the Doprb1F primer and the restriction enzyme KpnI site that had been introduced into the Doprb1R primer. The DNA fragment comprising the prb1 internal sequence was introduced into the DNA fragment comprising the hygromycin-resistant gene marker to obtain the pdPRB1 plasmid.

(2) Preparation of a Strain in which the Prb1 Gene has been Interrupted

The pdPRB1 plasmid was introduced into the NBRC 10746 strain via electroporation. The pdPRB1 was digested with the restriction enzyme AgeI, followed by ethanol precipitation, and the resultant DNA (5 μg) was used. After electroporation had been conducted under the conditions described in Example 2, the resultant was applied onto the casamino acid-U-A agar medium (6.7 g/l yeast nitrogen base w/o amino acids, 0.5 g/l casamino acid, 20 g/l glucose, 20 mg/l L-tryptophan, and 20 g/l agar) containing hygromycin B at a final concentration of 200 μg/ml, and it was then allowed to proliferate at 28° C. for about 3 days. The proliferated transformants were allowed to proliferate again on the casamino acid-U-A agar medium containing hygromycin B at a final concentration of 200 μg/ml, and transformants into which the pdPRB1 plasmid had been introduced were selected via colony PCR. Some yeast strains that had proliferated on the casamino acid-U-A agar medium containing hygromycin B at a final concentration of 200 μg/ml were suspended in 10 μl of a 0.25% (w/v) SDS solution, 90 μl of sterile water was added thereto, and strains were then removed via centrifugation at 3,100×g and 4° C. for 5 minutes. The resulting supernatant as a DNA solution was inspected with the use of the M13 RV primer (5′-CAGGAAACAGCTATGAC-3′: SEQ ID NO: 21) designed within the PRB1 ORF sequence and the dprb1 check ry primer (5′-CTAATCGAACAAATCAGCAACC-3′: SEQ ID NO: 22) designed within the pdPRB1 sequence, and a strain in which DNA amplification of about 1.5 kbp was observed was identified. Also, the DNA solution was inspected with the use of the dprb1 check fw primer (5′-ATGAAGTTATCCCAGTCTGCTG-3′: SEQ ID NO: 23) designed within the prb1 ORF sequence and the Hyg-t primer (5′-CAAAGGAATAGATCCCCCAT-3′: SEQ ID NO: 24) designed in the hygromycin-resistant gene within the pdPRB1 sequence, and a strain in which DNA amplification of about 2 kbp was observed was identified. These identified strains were designated as strains in which the prb1 gene had been interrupted (prb1::hyg, dprb1 strain).

### [Example 6] Preparation of a Strain into which a Chaperone Gene has been Introduced and in which the Prb1 Gene has been Interrupted

The pdPRB1 plasmid was introduced into the NBRC10746+PEK strain described in Example 4 via electroporation. The pdPRB1 was digested with the restriction enzyme AgeI, followed by ethanol precipitation, and the resultant DNA (5 μg) was used. After electroporation had been conducted under the conditions described in Example 2, the resultant was applied onto the casamino acid-U-A agar medium (6.7 g/l yeast nitrogen base w/o amino acids, 0.5 g/l casamino acid, 20 g/l glucose, 20 mg/l L-tryptophan, and 20 g/l agar) containing hygromycin B at a final concentration of 200 μg/ml, and it was then allowed to proliferate at 28° C. for about 3 days. The proliferated transformants were allowed to proliferate again on the casamino acid-U-A agar medium containing hygromycin B at a final concentration of 200 μg/ml, and transformants into which the pdPRB1 plasmid had been introduced were selected via colony PCR. Some yeast strains that had proliferated on the casamino acid-U-A agar medium containing hygromycin B at a final concentration of 200 μg/ml were suspended in 10 μl of a 0.25% (w/v) SDS solution, 90 μl of sterile water was added thereto, and strains were then removed via centrifugation at 3,100×g and 4° C. for 5 minutes. The resulting supernatant as a DNA solution was inspected with the use of the M13 RV primer (5′-CAGGAAACAGCTATGAC-3′: SEQ ID NO: 21) designed within the prb1 ORF sequence and the dprb1 check ry primer (5′-CTAATCGAACAAATCAGCAACC-3′: SEQ ID NO: 22) designed within the pdPRB1 sequence, and a strain in which DNA amplification of about 1.5 kbp was observed was identified. Also, the DNA solution was inspected with the use of the dprb1 check fw primer (5′-ATGAAGTTATCCCAGTCTGCTG-3′: SEQ ID NO: 23) designed within the prb1 ORF sequence and the Hyg-t primer (5′-CAAAGGAATAGATCCCCCAT-3′: SEQ ID NO: 24) designed in the hygromycin-resistant gene within the pdPRB1 sequence, and a strain in which DNA amplification of about 2 kbp was observed was identified. These identified strains were designated as strains into which a chaperone had been introduced and the prb1 gene had been interrupted (ura3::pdi1/ero1/kar2 prb1::hyg, NBRC10746+PEK dprb1 strains).

### [Example 7] Preparation of a Strain in which the Aox1 Gene has been Disrupted and the Chaperone has been Introduced

The onaP11007 plasmid was introduced into the Δaox1 strain described in Example 3 via electroporation. The onaP11007 was digested with the restriction enzyme NotI, followed by ethanol precipitation, and the resultant DNA (1 μg) was used. After electroporation had been conducted under the conditions described in Example 2, the resultant was applied onto the casamino acid-U-A agar medium (6.7 g/l yeast nitrogen base w/o amino acids, 0.5 g/l casamino acid, 20 g/l glucose, 20 mg/l L-tryptophan, and 20 g/l agar), and it was then allowed to proliferate at 28° C. for about 3 days. The proliferated transformants were allowed to proliferate again on the casamino acid-U-A agar medium, and transformants into which a chaperone gene had been introduced were selected via colony PCR. Some yeast strains that had proliferated on the casamino acid-U-A agar medium were suspended in 10 μl of a 0.25% SDS solution, 90 μl of sterile water was added thereto, and strains were then removed via centrifugation at 3,100×g and 4° C. for 5 minutes.

The resulting supernatant as a DNA solution was inspected in the manner described below. That is, introduction of the pdi1 gene was confirmed by detecting amplification of a DNA fragment of about 1.6 kbp with the use of the GAPpforS-F primer (5′-GATCTCAGGCCGAGTCAAGAC-3′: SEQ ID NO: 13) and the OmPDI-END Rv primer (5′-TTACAACTCGTCGTGAGCC-3′: SEQ ID NO: 14) designed within the gap promoter sequence. Introduction of the ero1 gene was confirmed by detecting amplification of a DNA fragment of about 1.6 kbp with the use of the GAPpforS-F primer (5′-GATCTCAGGCCGAGTCAAGAC-3′: SEQ ID NO: 13) and the OmERO-END Rv primer (5′-TTATAGCTCCAAACGATACAG-3′: SEQ ID NO: 15) designed within the gap promoter sequence. Introduction of the kar2 gene was confirmed by detecting amplification of a DNA fragment of about 2 kbp with the use of the PGKp-END Fw primer (5′-TAAACACTAACGCCGCAT-3′: SEQ ID NO: 16) and the OmKar-END Rv primer (5′-TCACAGCTCATCATGATCC-3′: SEQ ID NO: 17) designed within the pgk promoter sequence. PCR was carried out using TaKaRa LA Taq™ with GC Buffer (RR02AG, TaKaRa Bio) to amplify a target fragment (a cycle of 94° C. for 30 seconds, 55° C. for 30 seconds, and 72° C. for 120 seconds was repeated 30 times). Transformants in which amplification of interest was observed were designated as strains in which the aox1 gene had been disrupted and the chaperone gene had been introduced (Δaox1, ura3::pdi1/ero1/kar2, Δaox1+PEK strains).

### [Example 8] Preparation of a Strain in which the Aox1 Gene has been Disrupted, the Chaperone has been Introduced, and the Prb1 Gene has been Interrupted

The pdPRB1 plasmid was introduced into the Δaox1+PEK strain described in Example 7 via electroporation. The pdPRB1 plasmid was digested with the restriction enzyme AgeI, followed by ethanol precipitation, and the resultant DNA (5 μg) was used. After electroporation had been conducted under the conditions described in Example 2, the resultant was applied onto the casamino acid-U-A agar medium (6.7 g/l yeast nitrogen base w/o amino acids, 0.5 g/l casamino acid, 20 g/l glucose, 20 mg/l L-tryptophan, and 20 g/l agar) containing hygromycin B at a final concentration of 200 μg/ml, and it was then allowed to proliferate at 28° C. for about 3 days. The proliferated transformants were allowed to proliferate again on the casamino acid-U-A agar medium containing hygromycin B at a final concentration of 200 μg/ml, and transformants into which the pdPRB1 plasmid had been introduced were selected via colony PCR. Some yeast strains that had proliferated on the casamino acid-U-A agar medium containing hygromycin B at a final concentration of 200 μg/ml were suspended in 10 μl of a 0.25% (w/v) SDS solution, 90 μl of sterile water was added thereto, and strains were then removed via centrifugation at 3,100×g and 4° C. for 5 minutes.

The resulting supernatant as a DNA solution was inspected with the use of the M13 RV primer (5′-CAGGAAACAGCTATGAC-3′: SEQ ID NO: 21) designed within the prb1 ORF sequence and the dprb1 check ry primer (5′-CTAATCGAACAAATCAGCAACC-3′: SEQ ID NO: 22) designed within the pdPRB1 sequence, and a strain in which DNA amplification of about 1.5 kbp was observed was identified. Also, the DNA solution was inspected with the use of the dprb1 check fw primer (5′-ATGAAGTTATCCCAGTCTGCTG-3′: SEQ ID NO: 23) designed within the prb1 ORF sequence and the Hyg-t primer (5′-CAAAGGAATAGATCCCCCAT-3′: SEQ ID NO: 24) designed in the hygromycin-resistant gene within the pdPRB1 sequence, and a strain in which DNA amplification of about 2 kbp was observed was identified. These identified strains were designated as strains in which the aox1 gene had been disrupted, into which the chaperone had been introduced, and in which the prb1 gene had been interrupted (Δaox1, ura3::pdi1/ero1/kar2, prb1::hyg, Δaox1+PEK dprb1 strains).

### [Example 9] Amount of KEX2 Protein Secretion Induced by Methanol at Deep Well Plate Scale

### (1) Preparation of KEX2 Expression Plasmid

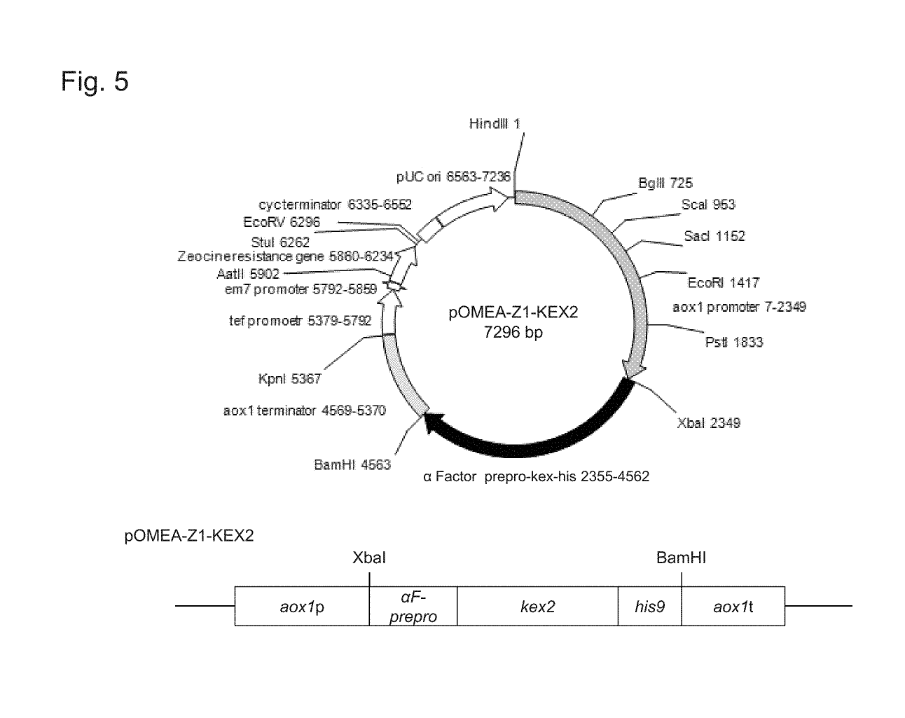

The KEX2 protein derived from S. cerevisiae (GenBank Accession Number M22870.1) comprises an amino acid sequence of 814 amino acid residues (SEQ ID NO: 33). In order to express an α-factor pre-sequence, an α-factor pro-sequence, KEX2 (amino acids 24 to 660 of SEQ ID NO: 33), and His9-tag with 9 His tags in the form of a fusion protein, the base sequence as shown in SEQ ID NO: 25 was artificially synthesized at Life technologies in accordance with the codon usage frequency of P. pastoris. A vector comprising the artificially synthesized base sequence was digested with the restriction enzymes XbaI and BamHI, and the resultant was introduced into a fragment obtained by digesting pOMEA-Z1 prepared in Example 1 with the restriction enzymes XbaI and BamHI. The resulting plasmid was designated as the kex2 expression plasmid (pOMEA-Z1-KEX2) (FIG. 5).

### (2) Preparation of KEX2-Producing Strain

(2-1) Preparation of KEX2-Producing Strain Derived from Chaperone-Introduced Strain

The kex2 expression plasmid was introduced into the NBRC10746+PEK strain described in Example 4 via electroporation. The kex2 expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium (6.7 g/l yeast nitrogen base w/o amino acids, 0.5 g/l casamino acid, 20 g/l glucose, 20 mg/l L-tryptophan, and 20 g/l agar) containing Zeocin at a final concentration of 200 μg/ml, and it was then allowed to proliferate at 28° C. for about 3 days. The proliferated transformants were allowed to proliferate again on the casamino acid-U-A agar medium containing Zeocin at a final concentration of 200 m/ml.

Strains capable of high levels of production of KEX2 were selected by conducting culture in the manner described below. A 2×YP-P6-dp medium [wherein the 2×YP-P6-dp medium was prepared by dissolving 20 g of a yeast extract and 40 g of peptone in 900 ml of pure water, subjecting the solution to high-pressure steam sterilization, and adding 100 ml of a separately sterilized 10× phosphate buffer (pH 6.0) (1 M KH₂PO₄, 0.15 M (NH₄)₂SO₄, 0.355 N KOH), 10 ml of a separately sterilized 50% glucose solution, and 6.25 ml of separately sterilized 80% glycerin] was used. The 2×YP-P6-dpn medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c and 10% (v/v) methanol [wherein the 2×YP-P6-dpn medium was prepared by dissolving 20 g of a yeast extract and 40 g of peptone in 900 ml of pure water, subjecting the solution to high-pressure steam sterilization, and adding 100 ml of a separately sterilized 10× phosphate buffer (pH 6.0) (1 M KH₂PO₄, 0.15 M (NH₄)₂SO₄, 0.355 N KOH)] was added, and 100 μl of the 2×YP-P6-dpn medium containing 40 μM rhodanine-3-acetic acid derivative 1c and 10% (v/v) methanol was further added 3 days after the initiation of culture. The strains were removed from the culture solution via centrifugation at 3,100×g and 4° C. for 5 minutes 4 days after the initiation of culture, and the resulting culture supernatant was designated as a KEX2-producing sample. Quantitative assays of the KEX2 that had been secreted and produced were performed in accordance with the dot blot technique. An SDS-PAGE buffer (SDS/β-mercaptoethanol) was added, the reaction was allowed to proceed at 100° C. for 5 minutes, 1 μl of the resulting culture supernatant was added dropwise to a nitrocellulose membrane to adsorb the proteins in the culture supernatant, and the KEX2-producing strain was selected via luminescence detection using the ECL Select™ Western Blotting Detection Reagent (RPN2235, GE Healthcare) and the peroxidase-labeled penta-His-specific antibody (Penta-His HRP Conjugate Kit, 34460, QIAGEN). The selected KEX2-producing strain was designated as the KEX2-producing strain derived from the chaperone-introduced strain (ura3::pdi1/ero1/kar2, NBRC10746+PEK strain).

(2-2) Preparation of KEX2-Producing Strain Derived from a Strain into which a Chaperone had been Introduced and in which the Prb1 Gene had been Interrupted

The kex2 expression plasmid was introduced into the NBRC10746+PEK dprb1 strain described in Example 6 via electroporation. The kex2 expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium containing Zeocin at a final concentration of 200 μg/ml and hygromycin B at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 μg/ml and hygromycin B at a final concentration of 200 m/ml.

Strains capable of high levels of production of KEX2 were selected by conducting culture in the manner described below. The 2×YP-P6-dpn medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c and 10% (v/v) methanol was added, and 100 μl of the 2×YP-P6-dpn medium containing 40 μM rhodanine-3-acetic acid derivative 1c and 10% (v/v) methanol was further added 3 days after the initiation of culture. The strains were removed from the culture solution via centrifugation at 3,100×g and 4° C. for 5 minutes 4 days after the initiation of culture, and the resulting culture supernatant was designated as a KEX2-producing sample. Quantitative assays of the KEX2 that had been secreted and produced were performed in accordance with the dot blot technique. An SDS-PAGE buffer (SDS/β-mercaptoethanol) was added, the reaction was allowed to proceed at 100° C. for 5 minutes, 1 μl of the resulting culture supernatant was added dropwise to a nitrocellulose membrane to adsorb the proteins in the culture supernatant, and the KEX2-producing strain was selected via luminescence detection using the ECL Select™ Western Blotting Detection Reagent (RPN2235, GE Healthcare) and the peroxidase-labeled penta-His-specific antibody (Penta-His HRP Conjugate Kit, 34460, QIAGEN). The selected KEX2-producing strain was designated as the KEX2-producing strain derived from the strain into which a chaperone had been introduced and in which the prb1 gene had been interrupted (ura3::pdi1/ero1/kar2, prb1::hyg, NBRC10746+PEK dprb1).

(2-3) Preparation of KEX2-Producing Strain Derived from a Strain in which the Aox1 Gene had been Disrupted and into which the Chaperone had been Introduced

The kex2 expression plasmid was introduced into the Δaox1+PEK strain described in Example 7 via electroporation. The kex2 expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium containing Zeocin at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 μg/ml.

Strains capable of high levels of production of KEX2 were selected by conducting culture in the manner described below. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c, 5% (w/v) glycerin, and 5% (v/v) methanol was added, and 100 μl of the 2×YP-P6-dpn medium containing 40 μM rhodanine-3-acetic acid derivative 1c and 5% (w/v) glycerin was further added 3 days after the initiation of culture. The strains were removed from the culture solution via centrifugation at 3,100×g and 4° C. for 5 minutes 4 days after the initiation of culture, and the resulting culture supernatant was designated as a KEX2-producing sample. Quantitative assays of the KEX2 that had been secreted and produced were performed in accordance with the dot blot technique. The Tris-SDS β-ME sample treatment solution (5 μl, 423437, Cosmo Bio, Co., Ltd.) was added to 5 μl of the culture supernatant, 1 μl of the culture supernatant resulting from a reaction conducted at 100° C. for 5 minutes was added dropwise to a nitrocellulose membrane to adsorb the proteins in the culture supernatant, and the KEX2-producing strain was selected via luminescence detection using the ECL Select™ Western Blotting Detection Reagent (RPN2235, GE Healthcare) and the peroxidase-labeled penta-His-specific antibody (Penta-His HRP Conjugate Kit, 34460, QIAGEN). The selected KEX2-producing strain was designated as the KEX2-producing strain derived from the strain in which the aox1 gene had been disrupted and the chaperone had been introduced (Δaox1, ura3::pdi1/ero1/kar2, Δaox1+PEK strain).

(2-4) Preparation of KEX2-Producing Strain Derived from a Strain in which the Aox1 Gene had been Disrupted, into which the Chaperone had been Introduced, and in which the Prb1 Gene had been Interrupted

The kex2 expression plasmid was introduced into the Δaox1+PEK dprb1 strain described in Example 8 via electroporation. The kex2 expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium containing Zeocin at a final concentration of 200 μg/ml and hygromycin B at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 μg/ml and hygromycin B at a final concentration of 200 m/ml.

Strains capable of high levels of production of KEX2 were selected by conducting culture in the manner described below. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c, 5% (w/v) glycerin, and 5% (v/v) methanol was added, and 100 μl of the 2×YP-P6-dpn medium containing 40 μM rhodanine-3-acetic acid derivative 1c and 5% glycerin was further added 3 days after the initiation of culture. The strains were removed from the culture solution via centrifugation at 3,100×g and 4° C. for 5 minutes 4 days after the initiation of culture, and the resulting culture supernatant was designated as a KEX2-producing sample. Quantitative assays of the KEX2 that had been secreted and produced were performed in accordance with the dot blot technique. The Tris-SDS β-ME sample treatment solution (5 μl, 423437, Cosmo Bio, Co., Ltd.) was added to 5 μl of the culture supernatant, 1 μl of the culture supernatant resulting from a reaction conducted at 100° C. for 5 minutes was added dropwise to a nitrocellulose membrane to adsorb the proteins in the culture supernatant, and the KEX2-producing strain was selected via luminescence detection using the ECL Select™ Western Blotting Detection Reagent (RPN2235, GE Healthcare) and the peroxidase-labeled penta-His-specific antibody (Penta-His HRP Conjugate Kit, 34460, QIAGEN). The selected KEX2-producing strain was designated as the KEX2-producing strain derived from the strain in which the aox1 gene had been disrupted, into which the chaperone had been introduced, and in which the prb1 gene had been interrupted (Δaox1, ura3::pdi1/ero1/kar2, prb1::hyg, Δaox1+PEK dprb1 strain).

### (3) Comparison of Secretory Production Amounts of KEX2

The KEX2-secreting and producing strains obtained above were applied onto the casamino acid-U-A agar medium containing Zeocin at a final concentration of 200 μg/ml (in the case of dprb1-derived strain, containing Zeocin at a final concentration of 200 μg/ml and hygromycin B at a final concentration of 200 μg/ml), and the strains were allowed to proliferate at 28° C. for about 2 days. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days. In the case of the NBRC10746+PEK- and NBRC10746+PEK dprb1-derived strains, thereafter, 100 μl of the 2×YP-P6-dpn medium containing 40 μM of rhodanine-3-acetic acid derivative 1c and 10% (v/v) methanol was added, and 100 μl of the 2×YP-P6-dpn medium containing 40 μM of rhodanine-3-acetic acid derivative 1c and 10% (v/v) methanol was further added 3 days after the initiation of culture. In the case of the Δaox1+PEK- and Δaox1+PEK dprb1-derived strains, 100 μl of the 2×YP-P6-dpn medium containing 40 μM rhodanine-3-acetic acid derivative 1c, 5% (w/v) glycerin, and 5% (v/v) methanol was added, and 100 μl of the 2×YP-P6-dpn medium containing 40 μM rhodanine-3-acetic acid derivative 1c and 5% (w/v) glycerin was further added 3 days after the initiation of culture. The strains were removed from the culture solution via centrifugation at 3,100×g and 4° C. for 5 minutes 4 days after the initiation of culture, and the resulting culture supernatant was designated as a KEX2-producing sample.

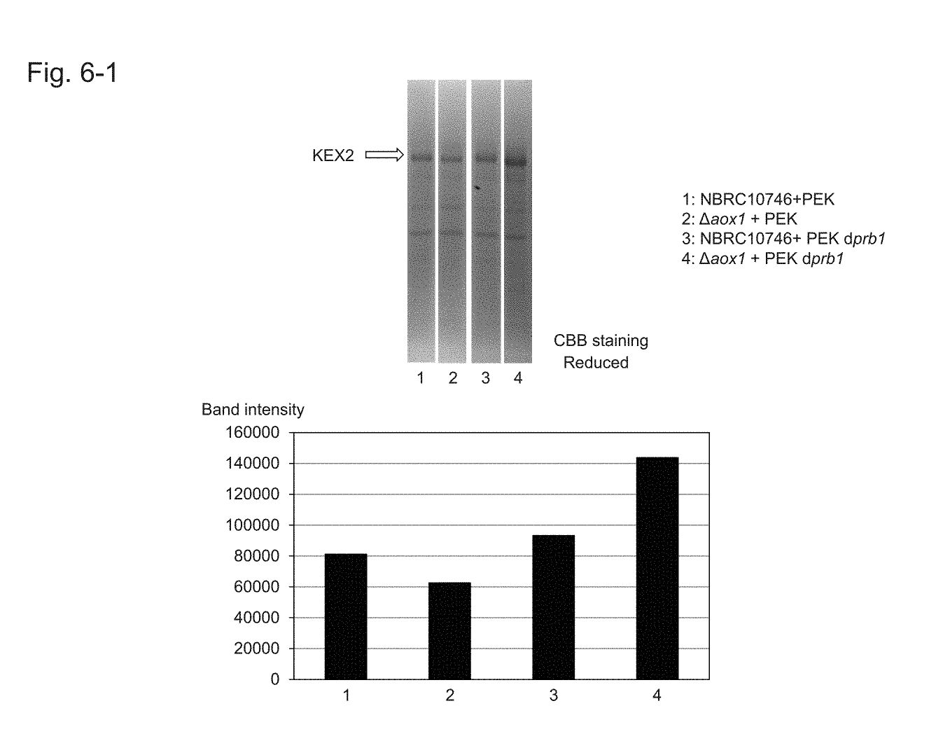

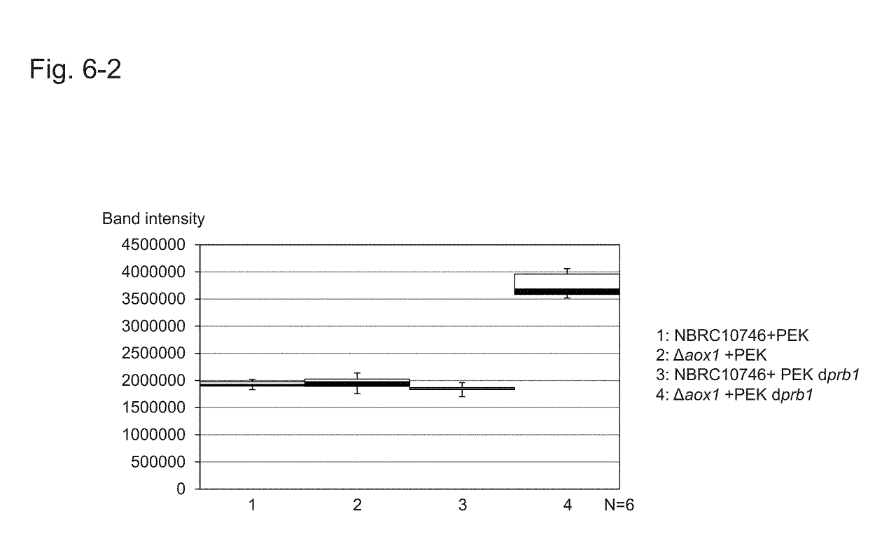

The KEX2-producing samples were compared by removing the N-linked sugar chain added to the produced KEX2 with the aid of a sugar chain cleavage enzyme (Endo H, P0702S, NEW ENGLAND Bio Labs) and observing the band intensities of the samples via SDS-PAGE/CBB staining. Band intensity was determined by photographing the SDS-PAGE gel using a Light-Capture (ATTO) and analyzing the photographs using analytical software (Cool Saver 2, Version 1.01.1058, ATTO). As shown in FIG. 6-1, the control sample (NBRC10746+PEK strain) exhibited a band intensity of 81178, and the NBRC10746+PEK dprb1 strain exhibited a band intensity of 93277. That is, the NBRC10746+PEK dprb1 strain exhibited improvement in secretory production that was about 1.1 times greater than that of the control sample. Meanwhile, the Δaox1+PEK dprb1 strain exhibited a band intensity of 143819 with the addition of methanol at a lower concentration than that in the case of the NBRC10746+PEK strain and the NBRC10746+PEK dprb1 strain. That is, the Δaox1+PEK dprb1 strain exhibited improvement in secretory production that was about 1.8 times greater than that of the control sample. In particular, the Δaox1+PEK dprb1 strain exhibited high-level secretory productivity with the addition of methanol at a lower concentration than that in the case of the control sample.

### [Example 10] Comparison of KEX2 Protein Enzyme Activity

FIG. 6-2 shows enzyme activity of KEX2 secreted and produced in Example 9. Enzyme activity was evaluated by fluorescence detection of AMC (7-amino-4-methylcoumarin) released upon the reaction of KEX2 with Boc-Leu-Arg-Arg-MCA (4-methylcoumaryl-7-amide) (#3140-v, Peptide Institute, Inc.) as a substrate. The strains were removed from the culture supernatant via centrifugation at 3,100×g and 4° C. for 5 minutes, and the resulting culture supernatant was designated as a KEX2-producing sample. After the sample had been adequately diluted with 100 mM Tris (pH 7.0), 100 μl of the diluted sample was mixed with 100 μl of the substrate solution (400 mM Tris (pH 7.0), 2 mM CaCl₂, 0.2% lubrol, 100 μM BOC-Leu-Arg-Arg-MCA), the mixture was subjected to reaction at 28° C. for 30 minutes, and 50 μl of a reaction terminator (5 mM EGTA (Na)) was added to terminate the reaction. Thereafter, AMC was quantified using a fluorescence plate reader (excitation wavelength: 355 nm; measurement wavelength: 460 nm). The AMC standard sample (#3099-v, manufactured by Peptide Institute, Inc.) was diluted and subjected to detection in the same manner, so as to prepare a calibration curve, and the KEX2 protease activity in the sample was determined. A unit of KEX2 activity was defined as the KEX2 content that releases 1 pmol of AMC every minute under the reaction conditions described above. The Δaox1+PEK dprb1 strain exhibited improvement in enzyme activity that was •BR>•0.8 times greater than that of the control (the NBRC10746+PEK strain).

### [Example 11] Amount of HSA Protein Secretion Induced by Methanol at Deep Well Plate Scale

### (1) Preparation of Hsa Expression Plasmid

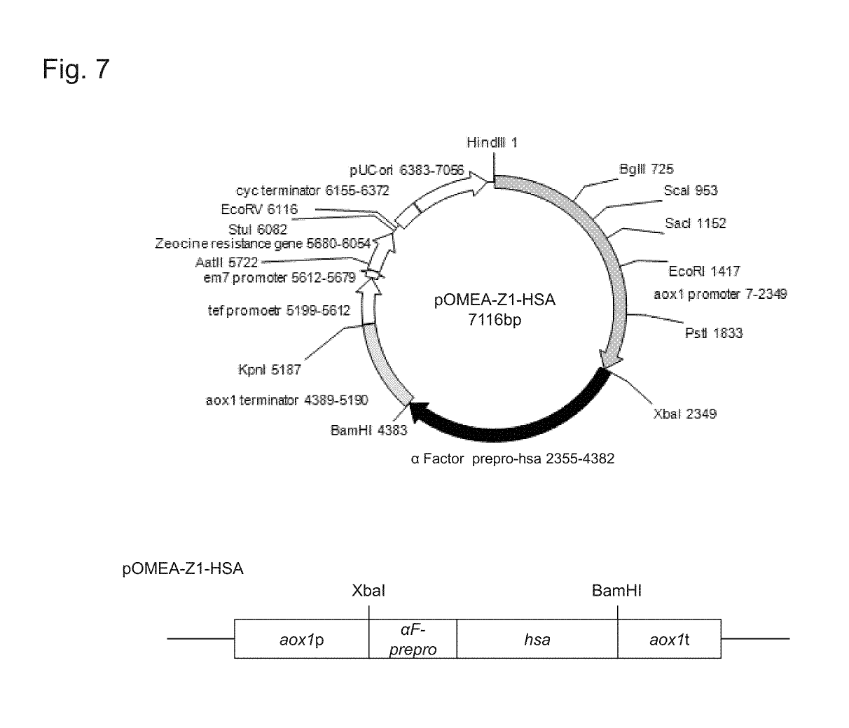

HSA (human serum albumin) (GenBank Accession Number NP000468) comprises an amino acid sequence of 609 amino acids (SEQ ID NO: 34). In order to express an α-factor pre-sequence and an α-factor pro-sequence derived from S. cerevisiae and HSA in the form of a fusion protein, DNA as shown in SEQ ID NO: 26 was artificially synthesized at Life technologies in accordance with the codon usage frequency of P. pastoris. A vector comprising the artificially synthesized DNA was digested with the restriction enzymes XbaI and BamHI, and the resultant was introduced into a fragment obtained by digesting pOMEA-Z1 with the restriction enzymes XbaI and BamHI. The resulting plasmid was designated as the hsa expression plasmid (pOMEA-Z1-HSA) (FIG. 7).

### (2) Preparation of HSA-Producing Strain

(2-1) Preparation of HSA-Producing Strain Derived from Chaperone-Introduced Strain

The hsa expression plasmid was introduced into the NBRC10746+PEK strain described in Example 4 via electroporation. The hsa expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium (6.7 g/l yeast nitrogen base W/O amino acids, 0.5 g/l casamino acid, 20 g/l glucose, 20 mg/l L-tryptophan, and 20 g/l agar) containing Zeocin at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 m/ml.

Strains capable of high levels of production of HSA were selected by conducting culture in the manner described below. A 2×YP-P6-dp medium [wherein the 2×YP-P6-dp medium was prepared by dissolving 20 g of a yeast extract and 40 g of peptone in 900 ml of pure water, subjecting the solution to high-pressure steam sterilization, and adding 100 ml of a separately sterilized 10× phosphate buffer (pH 6.0) (1 M KH₂PO₄, 0.15 M (NH₄)₂SO₄, 0.355 N KOH), 10 ml of a separately sterilized 50% glucose solution, and 6.25 ml of separately sterilized 80% glycerin] was used. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c and 10% (v/v) methanol [wherein the 2×YP-P6-dp medium was prepared by dissolving 20 g of a yeast extract and 40 g of peptone in 900 ml of pure water, subjecting the solution to high-pressure steam sterilization, and adding 100 ml of a separately sterilized 10× phosphate buffer (pH 6.0) (1 M KH₂PO₄, 0.15 M (NH₄)₂SO₄, 0.355 N KOH)] was added, and 100 μl of the 2×YP-P6-dpn medium containing 40 μM rhodanine-3-acetic acid derivative 1c and 10% (v/v) methanol was further added 3 days after the initiation of culture. The strains were removed from the culture solution via centrifugation at 3,100×g and 4° C. for 5 minutes 4 days after the initiation of culture, and the resulting culture supernatant was designated as a HSA-producing sample. Quantitative assays of the HSA that had been secreted and produced were performed in accordance with the dot blot technique. The Tris-SDS b-ME sample treatment solution (5 μl, 423437, Cosmo Bio, Co., Ltd.) was added to 5 μl of the culture supernatant, 1 μl of the culture supernatant resulting from a reaction conducted at 100° C. for 5 minutes was added dropwise to a nitrocellulose membrane to adsorb the proteins in the culture supernatant, and the HSA-producing strain was selected via luminescence detection using the ECL Select™ Western Blotting Detection Reagent (RPN2235, GE Healthcare) and the peroxidase-labeled human albumin-specific antibody (Goat anti-Human Albumin-HRP Conjugated) (A80-129P, BETHYL). The selected HSA-producing strain was designated as the HSA-producing strain derived from the chaperone-introduced strain (ura3::pdi1/ero1/kar2, NBRC10746+PEK strain).

(2-2) Preparation of HSA-Producing Strain Derived from a Strain into which a Chaperone had been Introduced and in which the Prb1 Gene had been Interrupted

The hsa expression plasmid was introduced into the NBRC10746+PEK dprb1 strain described in Example 6 via electroporation. The hsa expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium containing Zeocin at a final concentration of 200 μg/ml and hygromycin B at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 μg/ml and hygromycin B at a final concentration of 200 μg/ml.

Strains capable of high levels of production of HSA were selected by conducting culture in the manner described below. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c and 10% (v/v) methanol was added, and 100 μl of the 2×YP-P6-dpn medium containing 40 μM rhodanine-3-acetic acid derivative 1c and 10% (v/v) methanol was further added 3 days after the initiation of culture. The strains were removed from the culture solution via centrifugation at 3,100×g and 4° C. for 5 minutes 4 days after the initiation of culture, and the resulting culture supernatant was designated as a HSA-producing sample. Quantitative assays of the HSA that had been secreted and produced were performed in accordance with the dot blot technique. An SDS-PAGE buffer (SDS/β-mercaptoethanol) was added, the reaction was allowed to proceed at 100° C. for 5 minutes, 1 μl of the resulting culture supernatant was added dropwise to a nitrocellulose membrane to adsorb the proteins in the culture supernatant, and the HSA-producing strain was selected via luminescence detection using the ECL Select™ Western Blotting Detection Reagent (RPN2235, GE Healthcare) and the peroxidase-labeled human albumin-specific antibody (Goat anti-Human Albumin-HRP Conjugated) (A80-129P, BETHYL). The selected HSA-producing strain was designated as the HSA-producing strain derived from the strain into which a chaperone had been introduced and the prb1 gene had been interrupted (ura3::pdi1/ero1/kar2, prb1::hyg, NBRC10746+PEK dprb1).

(2-3) Preparation of HSA-Producing Strain Derived from a Strain in which the Aox1 Gene had been Disrupted and into which the Chaperone had been Introduced

The hsa expression plasmid was introduced into the Δaox1+PEK strain described in Example 7 via electroporation. The hsa expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium containing Zeocin at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 μg/ml.

Strains capable of high levels of production of HSA were selected by conducting culture in the manner described below. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c, 5% (w/v) glycerin, and 5% (v/v) methanol was added, and 100 μl of the 2×YP-P6-dpn medium containing 40 μM rhodanine-3-acetic acid derivative 1c and 5% (w/v) glycerin was further added 3 days after the initiation of culture. The strains were removed from the culture solution via centrifugation at 3,100×g and 4° C. for 5 minutes 4 days after the initiation of culture, and the resulting culture supernatant was designated as a HSA-producing sample. Quantitative assays of the HSA that had been secreted and produced were performed in accordance with the dot blot technique. The Tris-SDS β-ME sample treatment solution (5 μl, 423437, Cosmo Bio, Co., Ltd.) was added to 5 μl of the culture supernatant, 1 μl of the culture supernatant resulting from a reaction conducted at 100° C. for 5 minutes was added dropwise to a nitrocellulose membrane to adsorb the proteins in the culture supernatant, and the HSA-producing strain was selected via luminescence detection using the ECL Select™ Western Blotting Detection Reagent (RPN2235, GE Healthcare) and the peroxidase-labeled human albumin-specific antibody (Goat anti-Human Albumin-HRP Conjugated) (A80-129P, BETHYL). The selected HSA-producing strain was designated as the HSA-producing strain derived from the strain in which the aox1 gene had been disrupted and into which the chaperone had been introduced (Δaox1, ura3::pdi1/ero1/kar2, Δaox1+PEK strain).

(2-4) Preparation of HSA-Producing Strain Derived from a Strain in which the Aox1 Gene had been Disrupted, into which the Chaperone had been Introduced, and in which the Prb1 Gene had been Interrupted

The hsa expression plasmid was introduced into the Δaox1+PEK dprb1 strain described in Example 8 via electroporation. The hsa expression plasmid was digested with the restriction enzyme BglII, followed by ethanol precipitation, and the resultant DNA (1 μg) was used. After electroporation had been conducted under the conditions described in Example 2, the resultant was applied onto the casamino acid-U-A agar medium containing Zeocin at a final concentration of 200 μg/ml and hygromycin B at a final concentration of 200 μg/ml, and it was then allowed to proliferate at 28° C. for about 2 or 3 days. The proliferated transformants were allowed to proliferate again on the casamino acid-U-A agar medium containing Zeocin at a final concentration of 200 μg/ml and hygromycin B at a final concentration of 200 μg/ml.

Strains capable of high levels of production of HSA were selected by conducting culture in the manner described below. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days, 100 μl of the 2×YP-P6-dpn medium containing 40 μM rhodanine-3-acetic acid derivative 1c, 5% (w/v) glycerin, and 5% (v/v) methanol was added, and 100 μl of the 2×YP-P6-dpn medium containing 40 μM rhodanine-3-acetic acid derivative 1c and 5% glycerin was further added 3 days after the initiation of culture. The strains were removed from the culture solution via centrifugation at 3,100×g and 4° C. for 5 minutes 4 days after the initiation of culture, and the resulting culture supernatant was designated as a HSA-producing sample. Quantitative assays of the HSA that had been secreted and produced were performed in accordance with the dot blot technique. An SDS-PAGE buffer (SDS/β-mercaptoethanol) was added, the reaction was allowed to proceed at 100° C. for 5 minutes, 1 μl of the resulting culture supernatant was added dropwise to a nitrocellulose membrane to adsorb the proteins in the culture supernatant, and the HSA-producing strain was selected via luminescence detection using the ECL Select™ Western Blotting Detection Reagent (RPN2235, GE Healthcare) and the peroxidase-labeled human albumin-specific antibody (Goat anti-Human Albumin-HRP Conjugated) (A80-129P, BETHYL). The selected HSA-producing strain was designated as the HSA-producing strain derived from the strain in which the aox1 gene had been disrupted, into which the chaperone had been introduced, and in which the prb1 gene had been interrupted (Δaox1, ura3::pdi1/ero1/kar2, prb1::hyg, Δaox1+PEK dprb1 strain).

### (3) Comparison of Secretory Production Amounts of HSA

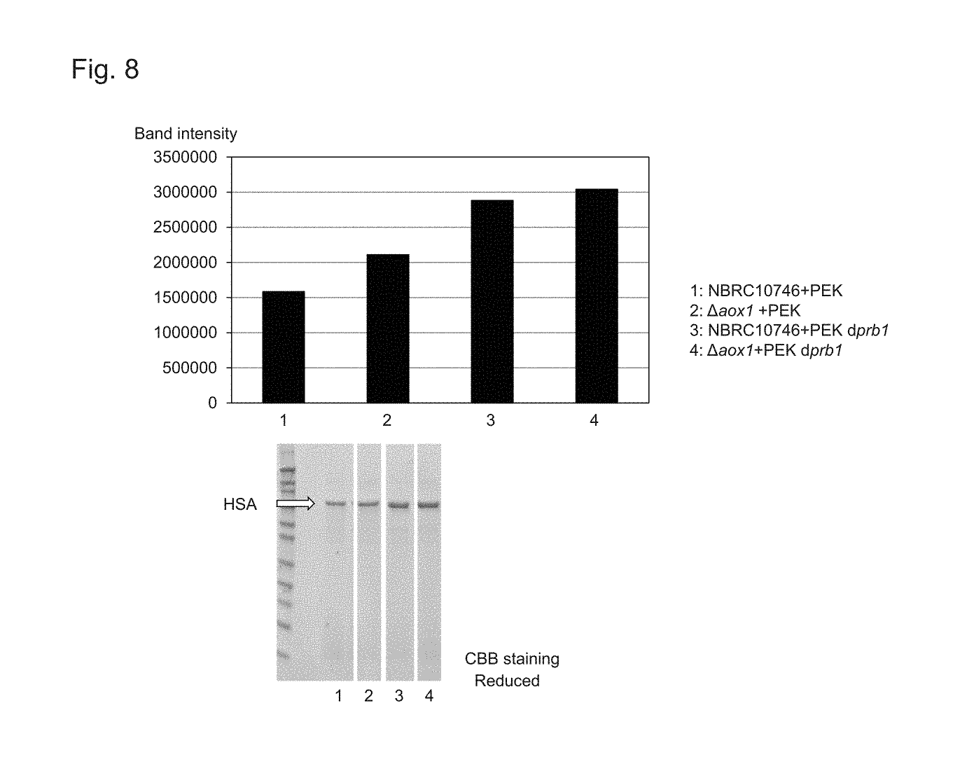

The HSA-secreting and producing strains obtained above were applied onto the casamino acid-U-A agar medium containing Zeocin at a final concentration of 200 μg/ml (in the case of the dprb1-derived strain, containing Zeocin at a final concentration of 200 μg/ml and hygromycin B at a final concentration of 200 μg/ml), and the strains were allowed to proliferate at 28° C. for about 2 days. The 2×YP-P6-dp medium (800 μl) was introduced into a 96-deep well plate (780271, Greiner), the strains were introduced thereinto using a toothpick, and the upper part of the plate was sealed with CO₂ permeable plate seal (676051, Greiner). Culture was conducted at an agitation speed of 310 rpm, an amplitude of 25 mm, and a temperature of 28° C. for 2 days. In the case of the NBRC10746+PEK- and NBRC10746+PEK dprb1-derived strains, 100 μl of the 2×YP-P6-dpn medium containing 40 μM rhodanine-3-acetic acid derivative 1c and 10% (v/v) methanol was added, and 100 μl of the 2×YP-P6-dpn medium containing 40 μM rhodanine-3-acetic acid derivative 1c and 10% (v/v) methanol was further added 3 days after the initiation of culture. In the case of the Δaox1+PEK− and Δaox1+PEK dprb1-derived strains, 100 μl of the 2×YP-P6-dpn medium containing 40 μM rhodanine-3-acetic acid derivative 1c, 5% (w/v) glycerin, and 5% (v/v) methanol was added, and 100 μl of the 2×YP-P6-dpn medium containing 40 μM rhodanine-3-acetic acid derivative 1c and 5% (w/v) glycerin was further added 3 days after the initiation of culture. The strains were removed from the culture solution via centrifugation at 3,100×g and 4° C. for 5 minutes 4 days after the initiation of culture, and the resulting culture supernatant was designated as a HSA-producing sample. The samples were compared based on the band intensity obtained from SDS-PAGE/CBB staining. Band intensity was determined by photographing the SDS-PAGE gel using a Light-Capture (ATTO) and analyzing the photographs using analytical software (Cool Saver 2, Version 1.01.1058, ATTO). As shown in FIG. 8, the control sample (NBRC10746+PEK strain) exhibited a band intensity of 1585061, and the NBRC10746+PEK dprb1 strain exhibited a band intensity of 2882752. That is, the NBRC10746+PEK dprb1 strain exhibited improvement in secretory production that was about 1.8 times greater than that of the control sample. The Δaox1+PEK strain exhibited a band intensity of 2111007, which is 1.3 times greater than that of the control sample. The Δaox1+PEK dprb1 strain exhibited a band intensity of 3041627, which is 1.9 times greater than that of the control sample and the greatest improvement among these strains. The Δaox1+PEK strain, especially, the Δaox1+PEK dprb1 strain exhibited high secretory productivity with the addition of methanol at a lower concentration than the case of the control sample (i.e., the NBRC10746+PEK strain).

### [Example 12] Amount of Secretion and Production of HSA Protein Induced by Methanol at 3-L Aeration-Agitation Culture Scale

The HSA-producing strains obtained in Example 11 (NBRC10746+PEK strain and Δaox1+PEK dprb1 strain) were compared in terms of the secretory production amount of the HSA protein induced by methanol at 3-L aeration-agitation culture scale. 3-L aeration-agitation culture was conducted in the manner described below. The HSA-producing strains obtained in Example 11 (NBRC10746+PEK strain and Δaox1+PEK dprb1 strain) were applied onto the casamino acid-U-A agar medium (6.7 g/l yeast nitrogen base W/O amino acids, 0.5 g/l casamino acid, 20 g/l glucose, 20 mg/l L-tryptophan, and 20 g/l agar) containing Zeocin at a final concentration of 200 μg/ml (in the case of the dprb1-derived strain, containing Zeocin at a final concentration of 200 μg/ml and hygromycin B at a final concentration of 200 μg/ml), and the strains were allowed to proliferate at 28° C. for about 2 days. The proliferated strains were inoculated into a 50-ml polypropyrene centrifuge tube (227241, Greiner) containing 5 ml of the casamino acid-U-A agar medium (6.7 g/l yeast nitrogen base W/O amino acids, 0.5 g/l casamino acid, 20 g/l glucose, and 20 mg/l L-tryptophan) containing Zeocin at a final concentration of 25 μg/ml (in the case of the dprb1-derived strain, containing Zeocin at a final concentration of 25 μg/ml and hygromycin B at a final concentration of 50 μg/ml), and the upper part of the centrifuge tube was sealed with plate seal (676051, Greiner). The reciprocal shake culture was conducted at an agitation speed of 250 rpm, an amplitude of 25 mm, and a temperature of 28° C. for 24 hours, and the resulting culture solution was designated as the first-type culture solution.

Subsequently, 10 ml of the first-type culture solution was inoculated into a 500-ml baffle flask (355123, BD Falcon) containing 40 ml of the 2×YP-P6 seed medium containing Zeocin at a final concentration of 25 μg/ml (in the case of the dprb1-derived strain, containing Zeocin at a final concentration of 25 μg/ml and hygromycin B at a final concentration of 50 μg/ml) [wherein the 2×YP-P6 seed medium was prepared by dissolving 20 g of a yeast extract and 40 g of peptone in 900 ml of pure water, subjecting the solution to high-pressure steam sterilization, and adding 100 ml of a separately sterilized 10× phosphate buffer (pH 6.0) (1 M KH₂PO₄, 0.15 M (NH₄)₂SO₄, 0.355 N KOH), 12.5 ml of a separately sterilized 50% glucose solution, and 62.5 ml of separately sterilized 80% glycerin] was applied onto the medium containing Zeocin at a final concentration of 25 μg/ml and hygromycin B at a final concentration of 50 μg/ml), and the upper part was sealed with plate seal (676051, Greiner). The reciprocal shake culture was conducted at an agitation speed of 180 rpm, an amplitude of 50 mm, and a temperature of 28° C. for 24 hours, and the resulting culture solution was designated as the second-type culture solution.

Subsequently, 60 ml of the second-type culture solution was inoculated into a 3-liter jar fermentor (BMS 03PI and BMS-03PII; ABLE Corporation) containing the 3×YP-P6 medium, which was prepared by dissolving 36 g of a yeast extract and 72 g of peptone in 1080 ml of pure water, subjecting the solution to high-pressure steam sterilization, and adding 120 ml of a separately sterilized 10× phosphate buffer (pH 6.0) (1 M KH₂PO₄, 0.15 M (NH₄)₂SO₄, 0.355 N KOH), 12 ml of a separately sterilized 50% (w/v) glucose solution, 60 ml of separately sterilized 80% (w/v) glycerin, and 100 μg of a defoaming agent (CB-442) (1.2 ml of 50 mg/ml hygromycin B was added in the case of the dprb1-derived strain). Culture was conducted at the culture temperature of 28° C., the internal pressure of 0.1 MPa, and DO of 2 ppm (automatically regulated via agitation). The feeding of an aqueous solution of 50% (w/v) glycerin was initiated 8 hours after the initiation of culture and the feeding was continued at 3 m/hr up to 24 hours after the initiation of culture. After the depletion of glycerin from the medium was confirmed, 780 μl of the 40 mM rhodanine-3-acetic acid derivative 1c was added.

Subsequently, methanol-induced culture was conducted in the manner described below. The control sample (NBRC10746+PEK strain: Jar1) was subjected to methanol-induced culture in a medium prepared by adding 32 ml of sterile water and 12 ml of 100% methanol to the medium that was confirmed to have been deprived of glycerin. The addition of 20 ml of sterile water and 86 ml of 100% methanol per day was initiated 2 hours after the initiation of methanol-induced culture and continued until the end of the culture period. The NBRC10746+PEK strain (Jar2) and the Δaox1+PEK dprb1 strain (Jar3) were subjected to pH-controlled, nitrogen-source-fed, and methanol-induced culture in the manner described below. The NBRC10746+PEK strain (Jar2) was subjected to methanol-induced culture in the medium that was confirmed to have been deprived of glycerin by adding 32 ml of 10% (w/v) L-histidine monohydrochloride monohydrate, initiating automatic control of the pH level with 14% (v/v) ammonia water, and adding 12 ml of 100% methanol. The Δaox1+PEK dprb1 strain (Jar3) was subjected to methanol-induced culture in the medium that was confirmed to have been deprived of glycerin by adding 32 ml of 10% (w/v) L-histidine monohydrochloride monohydrate and 1.2 ml of 50 mg/ml hygromycin B, initiating automatic control of the pH level with 14% (v/v) ammonia water, and adding 6.5 ml of 100% methanol. In the case of the NBRC10746+PEK strain (Jar2), the pH level of the culture solution was adjusted to 6.75 three hours after the initiation of methanol-induced culture and it was adjusted to 7 six hours after the initiation of culture and thereafter. Immediately after the initiation of methanol-induced culture, the feeding of 20 ml of the nitrogen source feeding solution (300 g/l yeast extract and 80 g/l L-histidine monohydrochloride monohydrate) and 86 ml of 100% methanol per day was initiated and it was continued until the end of the culture period. In the case of the Δaox1+PEK dprb1 strain (Jar3), the pH level of the culture solution was adjusted to 6.75 three hours after the initiation of methanol-induced culture and it was adjusted to 7 six hours after the initiation of culture and thereafter. Further, the feeding of 20 ml of a nitrogen source feeding solution, 30 ml of 80% (w/v) glycerin, and 56 ml of sterile water per day was initiated immediately after the initiation of methanol-induced culture and it was continued until the end of the culture period. Also, 1.2 ml of 50 mg/ml hygromycin B and methanol at a final concentration of 0.2-0.5% (v/v) were intermittently fed per day from 24 hours after the initiation of methanol-induced culture. From 9 days after the initiation of methanol-induced culture, 100 ml of the culture was allowed to overflow every day.

The culture solutions were subjected to centrifugation at 3,000×g and 4° C. for 5 minutes to remove the strains, and the resulting culture supernatant was designated as a HSA-producing sample. Quantitative assays of HSA secreted and produced were carried out by comparing the samples based on the band intensity obtained by SDS-PAGE/CBB staining. Band intensity was determined by photographing the SDS-PAGE gel using a Light-Capture (ATTO) and analyzing the photographs using analytical software (Cool Saver 2, Version 1.01.1058, ATTO). HSA was quantified on the basis of the calibration curve indicating the band intensity of the Albumin Standard (232209, Thermo scientific) as the standard sample.

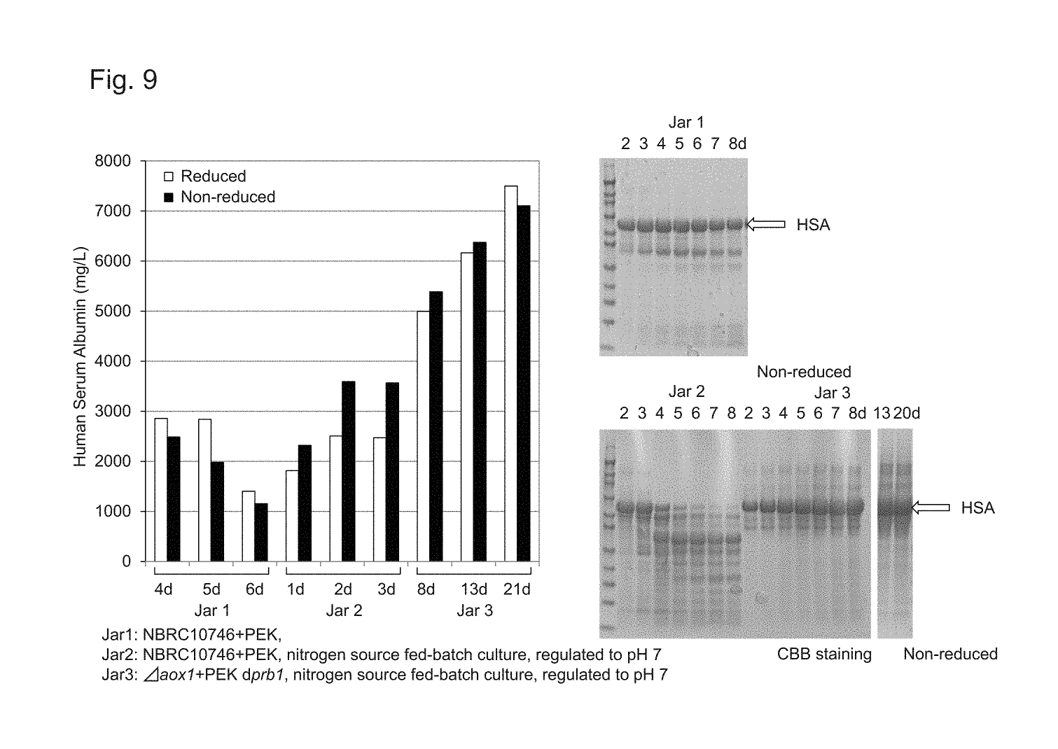

FIG. 9 shows the results of quantification of the secretory production amount of HSA. The control sample (NBRC10746+PEK strain: Jar1) exhibited the productivity peak 4 or 5 days after the initiation of methanol-induced culture. In the case of pH-controlled and nitrogen source fed-batch culture, the productivity peak of the NBRC10746+PEK strain (Jar2) was observed 2 or 3 days after the initiation of methanol-induced culture, and degradation of the secreted and produced HSA became significant 4 days after the initiation of culture and thereafter. In contrast, productivity of the Δaox1+PEK dprb1 strain (Jar3) was maintained up to 21 days after the initiation of culture, degradation was not substantially observed, and it was thus found to be a protein production system capable of long-term culture. In the case of pH-controlled and nitrogen source fed-batch culture, the amount of HSA secreted and produced by the NBRC10746+PEK strain (Jar2) was improved by about 1.4 times compared with the control sample (NBRC10746+PEK strain: Jar1) (improved from 2486 mg/l to 3594 mg/1). Secretory productivity was improved by about 2.9 times in the case of the Δaox1+PEK dprb1 strain (Jar3) (improved from 2486 mg/l to 7104 mg/1). The amount of methanol added when culturing the Δaox1+PEK dprb1 strain (Jar3) was reduced to about 1/17 (5.8%) that of the control sample (NBRC10746+PEK strain: Jar1) and the NBRC10746+PEK strain (Jar2) up to 7 days after the initiation of methanol-induced culture. Accordingly, the A aox1+PEK dprb1 strain (Jar3) that had been subjected to pH-controlled and nitrogen source fed-batch culture was found to have realized a significant reduction of the amount of methanol added and high-level secretory productivity, compared with the control sample (NBRC10746+PEK strain: Jar1) and the NBRC10746+PEK strain (Jar2).

### [Example 13] Secretory Productivity of HSA Protein Under Carbon Source-Starvation-Induction at 3-L Aeration-Agitation Culture Scale

The amount of the HSA protein secreted and produced by the HSA-producing strain derived from the Δaox1+PEK dprb1 strain obtained in Example 11 under carbon source-starvation-induction was compared with that under methanol-induction at the 3-L aeration-agitation culture scale. 3-L aeration-agitation culture was conducted in the manner described below. The HSA-producing strain derived from the Δaox1+PEK dprb1 strain obtained in Example 11 was applied onto the casamino acid-U-A agar medium (6.7 g/l yeast nitrogen base W/O amino acids, 0.5 g/l casamino acid, 20 g/l glucose, 20 mg/l L-tryptophan, and 20 g/l agar) containing Zeocin at a final concentration of 200 μg/ml and hygromycin B at a final concentration of 200 μg/ml, and the strains were allowed to proliferate at 28° C. for about 2 days. The proliferated strains were inoculated into a 50-ml polypropyrene centrifuge tube (227241, Greiner) containing 5 ml of the casamino acid-U-A agar medium (6.7 g/l yeast nitrogen base W/O amino acids, 0.5 g/l casamino acid, 20 g/l glucose, and 20 mg/l L-tryptophan) containing Zeocin at a final concentration of 25 μg/ml and hygromycin B at a final concentration of 50 μg/ml, and the upper part was sealed with plate seal (676051, Greiner). The reciprocal shake culture was conducted at an agitation speed of 250 rpm, an amplitude of 25 mm, and a temperature of 28° C. for 24 hours, and the resulting culture solution was designated as the first-type culture solution.

Subsequently, 10 ml of the first-type culture solution was inoculated into a 500-ml baffle flask (355123, BD Falcon) containing 40 ml of the 2×YP-P6 seed medium containing Zeocin at a final concentration of 31.25 μg/ml and hygromycin B at a final concentration of 62.5 μg/ml [wherein the 2×YP-P6 seed medium was prepared by dissolving 20 g of a yeast extract and 40 g of peptone in 900 ml of pure water, subjecting the solution to high-pressure steam sterilization, and adding 100 ml of a separately sterilized 10× phosphate buffer (pH 6.0) (1 M KH₂PO₄, 0.15 M (NH₄)₂SO₄, 0.355 N KOH), 12.5 ml of a separately sterilized 50% glucose solution, and 62.5 ml of separately sterilized 80% glycerin], and the upper part was sealed with plate seal (676051, Greiner). The reciprocal shake culture was conducted at an agitation speed of 180 rpm, an amplitude of 50 mm, and a temperature of 28° C. for 24 hours, and the resulting culture solution was designated as the second-type culture solution.

Subsequently, 75 ml of the second-type culture solution was inoculated into a 3-liter jar fermentor (BMS 03PI and BMS-03PII; ABLE Corporation) containing the 3×YP-P6 medium, which was prepared by dissolving 45 g of a yeast extract and 90 g of peptone in 1350 ml of pure water, subjecting the solution to high-pressure steam sterilization, and adding 150 ml of a separately sterilized 10× phosphate buffer (pH 6.0) (1 M KH₂PO₄, 0.15 M (NH₄)₂SO₄, 0.355 N KOH), 15 ml of a separately sterilized 50% (w/v) glucose solution, 75 ml of separately sterilized 80% (w/v) glycerin, 1.5 ml of 50 mg/ml hygromycin B, and 100 μg of a defoaming agent (CB-442). Culture was conducted at the culture temperature of 28° C., the internal pressure of 0.1 MPa, and DO of 2 ppm (automatically regulated via agitation). The feeding of an aqueous solution of 50% (w/v) glycerin was initiated 8 hours after the initiation of culture and the feeding was continued at 4.3 ml/hr up to 22 hours after the initiation of culture.

After the depletion of glycerin from the medium was confirmed, subsequently, methanol-induced culture, carbon source-starvation culture, and induction culture from the aox promoter were conducted in the manner described below. Methanol-induced culture was initiated in the medium that was confirmed to have been deprived of glycerin by adding 54 ml of sterile water, 40 ml of 10% (w/v) L-histidine monohydrochloride monohydrate, and 1.5 ml of 50 mg/ml of hygromycin B, regulating the pH level of the culture solution to 6.5 with 14% (v/v) ammonia water, and adding 8.5 ml of 100% methanol. Thereafter, the pH level was adjusted to 6.75 three hours after the initiation of methanol-induced culture and it was adjusted to 7 six and a half hours after the initiation of culture and thereafter. Immediately after the initiation of methanol-induced culture, the feeding of 12.5 ml of the nitrogen source feeding solution (300 g/l yeast extract and 80 g/l L-histidine monohydrochloride monohydrate) and 37.5 ml of 80% (w/v) glycerin per day was initiated, it was continued until the end of the culture period, and 5.1 to 5.7 ml of 100% methanol (final concentration: 0.25-0.35% (v/v)) was intermittently fed per day from 24 hours after the initiation of methanol-induced culture and thereafter. Further, 1.5 ml of 50 mg/ml hygromycin B was added 48, 96, and 144 hours after the initiation of methanol-induced culture.

Meanwhile, starvation-induced culture comprising carbon source depletion was initiated in the medium that was confirmed to have been deprived of glycerin by adding 40 ml of 10% (w/v) L-histidine monohydrochloride monohydrate and 1.5 ml of 50 mg/ml hygromycin B, and regulating the pH level to 6.5 with 14% (v/v) ammonia water (hereafter, it is referred to as “carbon source starvation-induced culture”). The pH level was adjusted to 6.75 three hours after the initiation of carbon source starvation-induced culture and it was adjusted to 7 six and a half hours after the initiation of culture and thereafter. Immediately after the initiation of carbon source starvation-induced culture, continuous feeding of 12.5 ml of the nitrogen source feeding solution (300 g/l yeast extract and 80 g/l L-histidine monohydrochloride monohydrate) and 92 ml of 65% (w/v) sorbitol per day was initiated. The nitrogen source feeding solution (12.5 ml) and 37.5 ml of 80% (w/v) glycerin were continuously fed per day from 24 hours after the initiation of methanol-induced culture up to the end of the culture period. Further, 1.5 ml of 50 mg/ml hygromycin B was intermittently fed per day 48, 96, and 144 hours after the initiation of carbon source starvation-induced culture.

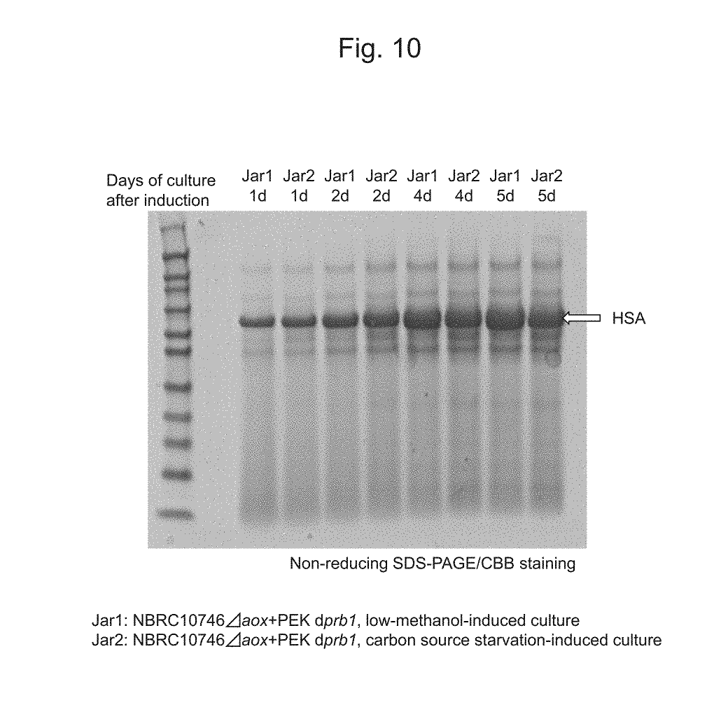

The culture solutions were subjected to centrifugation at 3,000×g and 4° C. for 5 minutes to remove the strains, and the resulting culture supernatant was designated as a HSA-producing sample. Quantitative comparison assays of HSA secreted and produced were carried out by comparing the samples based on the SDS-PAGE/CBB staining intensity. FIG. 10 shows the results of quantification of the secretory production amount of HSA. It was found that the productivity attained via carbon source starvation-induced culture was substantially equivalent to that attained via methanol-induced culture.

### INDUSTRIAL APPLICABILITY

The present invention is applicable to the field of production of protein pharmaceuticals, such as antigen or antibody.

All publications, patents, and patent applications cited herein are incorporated herein by reference in their entirety.

1. A transformed yeast into which a chaperone gene has been introduced and in which the aox1 gene has been disrupted.

2. The transformed yeast according to claim 1, wherein the chaperone gene is at least one gene selected from the group consisting of genes (a) to (d) below:
(a) a gene encoding PDI1, ERO1, Kar2, MPD1, SCJ1, EUG1, or HSP104 derived from Ogataea minuta (O. minuta);
(b) a gene encoding PDI1, MPD1, SCJ1, ERO1, FKB2, JEM1, LHS1, MPD2, ERJ5, or EUG1 derived from Saccharomyces cerevisiae (S. cerevisia);
(c) a gene encoding PDI, ERO1-Lα, ERO1-Lβ, or GRP78 derived from a human; and
(d) a gene exhibiting 95% or higher sequence homology to a base sequence of any of the genes (a) to (c).

3. The transformed yeast according to claim 1, wherein the chaperone gene is at least one gene selected from the group consisting of genes (a) to (g) below:
(a) a gene encoding PDI1 derived from O. minuta;
(b) a gene encoding ERO1 derived from O. minuta;
(c) a gene encoding Kar2 derived from O. minuta;
(d) a gene encoding PDI1 derived from S. cerevisiae;
(e) a gene encoding PDI derived from a human;
(f) a gene encoding ERO1 derived from a human; and
(g) a gene exhibiting 95% or higher sequence homology to a base sequence of any of the genes (a) to (f).

4. The transformed yeast according to claim 1, wherein the chaperone gene is any of the chaperone genes (a) to (g) below:
(a) a combination of a gene encoding PDI1, a gene encoding ERO1, and a gene encoding Kar2 derived from O. minuta;
(b) a combination of a gene encoding PDI1 and a gene encoding Kar2 derived from O. minuta;
(c) a combination of a gene encoding PDI derived from a human and a gene encoding ERO1 derived from O. minuta;
(d) a combination of a gene encoding PDI1 and a gene encoding ERO1 derived from O. minuta;
(e) a combination of a gene encoding PDI derived from a human, a gene encoding ERO1-Lβ derived from a human, and a gene encoding GRP78 derived from a human;
(f) a combination of a gene encoding PDI derived from a human, a gene encoding ERO1 derived from O. minuta, and a gene encoding GRP78 derived from a human; and
(g) a gene exhibiting 95% or higher sequence homology to a base sequence of any of the genes (a) to (f).

5. The transformed yeast according to claim 1, wherein the protease gene has been disrupted.

6. The transformed yeast according to claim 5, wherein the protease is a prb1 gene.

7. A transformed yeast into which a chaperone gene has been introduced and in which a protease gene has been disrupted.

8. The transformed yeast according to claim 7, wherein the protease is a prb1 gene.

9. The transformed yeast according to claim 1, wherein the yeast is a methhylotrophic yeast.

10. The transformed yeast according to claim 1, which comprises a gene encoding a target protein introduced thereinto.

11. Use of the transformed yeast according to claim 1 for the production of a target protein.

12. A method for producing a protein comprising culturing the transformed yeast according to claim 10 in a medium and sampling a target protein from the culture product.

13. The method for producing a protein according to claim 12, wherein culture is conducted under conditions in which protease activity is inhibited.

14. The method for producing a protein according to claim 12 or 13, wherein culture is conducted in a medium with a pH of 6.0 to 7.5.

15. The method for producing a protein according to claim 12, wherein a nitrogen source is added to the medium.

16. The method for producing a protein according to claim 12, wherein the amount of methanol added to the medium is 2% (v/v) or less.

17. A target protein produced by the method according to claim 12.

18. A method for producing a transformed yeast comprising step (i) in addition to either or both step (ii) and/or (iii):
(i) a step of introducing a chaperone gene into yeast; and
(ii) a step of disrupting the aox1 gene in yeast; and/or
(iii) a step of disrupting the prb1 gene in yeast.

19. The method of production according to claim 18, which further comprises a step of introducing a gene encoding a target protein.


SEQUENCE LISTING (76 sequences)

SEQ ID NO: 1 - DNA, 29 bp, Artificial
  primer
gcaagctttctttcgcaaacagctctttg

SEQ ID NO: 2 - DNA, 41 bp, Artificial Sequence
  primer
gaacccgggaacagaatctagattttttcgtaagtcgtaag

SEQ ID NO: 3 - DNA, 39 bp, Artificial Sequence
  primer
ctgttcccgggttcctggatccgagacggtgcccgactc

SEQ ID NO: 4 - DNA, 25 bp, Artificial Sequence
  primer
gcggtaccgttagtggtacgggcag

SEQ ID NO: 5 - DNA, 17 bp, Artificial Sequence
  primer
ggtaccagtactggaaa

SEQ ID NO: 6 - DNA, 34 bp, Artificial Sequence
  primer
cagataaacaggcgacttttcgggtcacgtgact

SEQ ID NO: 7 - DNA, 34 bp, Artificial Sequence
  primer
agtcacgtgacccgaaaagtcgcctgtttatctg

SEQ ID NO: 8 - DNA, 17 bp, Artificial Sequence
  primer
ccaaggaggaagaaatt

SEQ ID NO: 9 - DNA, 17 bp, Artificial Sequence
  primer
atcacaggaaagcgcat

SEQ ID NO: 10 - DNA, 17 bp, Artificial Sequence
  primer
attcgagcatcgccgtg

SEQ ID NO: 11 - DNA, 20 bp, Artificial Sequence
  primer
atggctattcctgacgaatt

SEQ ID NO: 12 - DNA, 22 bp, Artificial Sequence
  primer
ttagaatctagccagacccttc

SEQ ID NO: 13 - DNA, 21 bp, Artificial Sequence
  primer
gatctcaggccgagtcaagac

SEQ ID NO: 14 - DNA, 19 bp, Artificial Sequence
  primer
ttacaactcgtcgtgagcc

SEQ ID NO: 15 - DNA, 21 bp, Artificial Sequence
  primer
ttatagctccaaacgatacag

SEQ ID NO: 16 - DNA, 18 bp, Artificial Sequence
  primer
taaacactaacgccgcat

SEQ ID NO: 17 - DNA, 19 bp, Artificial Sequence
  primer
tcacagctcatcatgatcc

SEQ ID NO: 18 - DNA, 24 bp, Artificial Sequence
  primer
caagcttcgttggcagcagtggag

SEQ ID NO: 19 - DNA, 24 bp, Artificial Sequence
  primer
cggtacccgatggaatctcagaca

SEQ ID NO: 20 - DNA, 1384 bp, Ogataea minuta
caagcttcgttggcagcagtggaggccttggtcatcccgttatttgacgacttgccagcagagtttgcccttgttccaatggatgcgaaagcggaagtcatttctgacgttcctgtcgactcggccattagtgatgctcctatcgcggcactaaatgatgctccaagccctctcgtcacatcgctgatcgcatctcaaaatttgattccaaactcttatattgtcgttttcaagaatggcctagcttccggggcagttgacttccacatggagtggctcaaggaaacgcactcccaaaccctggctgctttgtctaaggacatgccagcagaagaattggccgccgaaggtttcgtttccgaaagcattgatcttactgaggtgtttagcatctccgatttgttcagtggatataccggatacttcccggagaaggtggttgacctcatcagaagacaccctgacgtggcgttcgttgagcaggactcgagagttttcgccgataagtcgtctactcaaaacggtgctccttggggtttgtctagaatctctcacagagagcctctcagtctcggcaatttcaacgagtacgtttacgacgatcttgctggagatggcgtcacggcttatgtcattgataccggtatcaatgtgaagcacgagcagttcggtggcagagcagagtggggtaagaccatcccaaccggtgatgatgatattgacggaaacggtcacggtactcactgcgctggtacaattggctcggaagattatggagtttctaagaactccaaaattgtcgcagtgaaggttttgagatctaacggttctggttccatgtctgacgtgatcaagggtgttgaattcgctgcaaatgatcacgttgccaagtctaaagccaagaaggacggtttcaagggatcgactgccaacatgtctttgggaggtggcaagtctcctgctcttgacttggctgtcaatgccgctgtcaaagctggtttacactttgctgttgccgctggtaacgacaatgctgacgcatgcaactattctcctgctgctgcagagaacgcagtcactgttggtgcgtccactttgtctgactctagagcttacttttccaactatggtaaatgtgttgacatttttgctccgggcttgaacatcctttccacctacataggttctgacactgccaccgccactctttctggtacatcgatggcctcccctcacgtttgtggtctgttgacctactttttgagcttgcaaccagaatcgtcgtcgttgttttcttcggcagctatctcccctgctcagctgaagaagaacctgatcaagtttggtacgaagaacgttttgtctgagattccatcgggtaccg

SEQ ID NO: 21 - DNA, 17 bp, Artificial Sequence
  primer
caggaaacagctatgac

SEQ ID NO: 22 - DNA, 22 bp, Artificial Sequence
  primer
ctaatcgaacaaatcagcaacc

SEQ ID NO: 23 - DNA, 22 bp, Artificial Sequence
  primer
atgaagttatcccagtctgctg

SEQ ID NO: 24 - DNA, 20 bp, Artificial Sequence
  primer
caaaggaatagatcccccat

SEQ ID NO: 25 - DNA, 2220 bp, Artificial Sequence
  synthetic nucleotide
tctagaatgagattcccatccatcttcaccgccgttttgttcgctgcttcctccgctttggctgctccagttaacaccaccaccgaggacgagaccgctcaaatcccagctgaggctgttatcggttactccgacttggagggtgacttcgacgttgctgttttgccattctccaactccaccaacaacggtttgttgttcatcaacaccaccatcgcttccatcgctgctaaggaggagggtgtttccttggagaagagagaggctgaggctcaacaaatcccattgaaggaccacacctccagacaatacttcgctgttgagtccaacgagaccttgtccagattggaggagatgcacccaaactggaagtacgagcacgacgttagaggtttgccaaaccactacgttttctccaaggagttgttgaagttgggtaagagatcctccttggaggagttgcaaggtgacaacaacgaccacatcttgtccgttcacgacttgttcccaagaaacgacttgttcaagagattgccagttccagctccaccaatggactcctccttgttgccagttaaggaggctgaggacaagttgtccatcaacgacccattgttcgagagacaatggcacttggttaacccatccttcccaggttccgacatcaacgttttggacttgtggtacaacaacatcaccggtgctggtgttgttgctgctatcgttgacgacggtttggactacgagaacgaggacttgaaggacaacttctgtgctgagggttcctgggacttcaacgacaacaccaacttgccaaagccaagattgtccgacgactaccacggtaccagatgtgctggtgagatcgctgctaagaagggtaacaacttctgtggtgttggtgttggttacaacgctaagatctccggtatcagaatcttgtccggtgacatcaccaccgaggacgaggctgcttccttgatctacggtttggacgttaacgacatctactcctgttcctggggtccagctgacgacggtagacacttgcaaggtccatccgacttggttaagaaggctttggttaagggtgttaccgagggtagagactccaagggtgctatctacgttttcgcttccggtaacggtggtaccagaggtgacaactgtaactacgacggttacaccaactccatctactccatcaccatcggtgctatcgaccacaaggacttgcacccaccatactccgagggttgttccgctgttatggctgttacctactcctccggttccggtgagtacatccactcctccgacatcaacggtagatgttccaactcccacggtggtacctccgctgctgctccattggctgctggtgtttacaccttgttgttggaggctaacccaaacttgacctggagagacgttcaatacttgtccatcttgtccgctgttggtttggagaagaacgctgacggtgactggagagactccgctatgggtaagaagtactcccacagatacggtttcggtaagatcgacgctcacaagttgatcgagatgtccaagacctgggagaacgttaacgctcaaacctggttctacttgccaaccttgtacgtttcccaatccaccaactccaccgaggagaccttggagtccgttatcaccatctccgagaagtccttgcaagacgctaacttcaagagaatcgagcacgttaccgttaccgttgacatcgacaccgagatcagaggtaccaccaccgttgacttgatctccccagctggtatcatctccaacttgggtgttgttagaccaagagacgtttcctccgagggtttcaaggactggaccttcatgtccgttgctcactggggtgagaacggtgttggtgactggaagatcaaggttaagaccaccgagaacggtcacagaatcgacttccactcctggagattgaagttgttcggtgagtccatcgactcctccaagaccgagaccttcgttttcggtaacgacaaggaggaggttgagccagctgctaccgagtccaccgtttcccaatactccgcttcctccacctccatctccatctccgctacctccacctcctccatctccatcggtgttgagacctccgctatcccacaaaccaccaccgcttccaccgacccacaccaccaccaccaccaccaccaccactaaggatcc

SEQ ID NO: 26 - DNA, 2040 bp, Artificial Sequence
  synthetic nucleotide
tctagaatgagattcccatccatcttcactgctgttttgttcgctgcttcttccgctttggctgctccagttaacactactactgaggacgagactgctcagattccagctgaagctgttattggttactccgacttggaaggtgacttcgacgttgctgttttgccattctccaactccactaacaacggtttgttgttcatcaacactacaatcgcttccattgctgctaaagaagagggagtttccttggagaagagagaagctgaggctgacgctcacaagtctgaagttgctcacagattcaaggacttgggtgaagagaacttcaaggctttggttttgatcgctttcgctcagtacttgcagcagtgtccattcgaggatcacgttaagttggttaacgaggttactgagttcgctaagacttgtgttgctgacgaatccgctgagaactgtgataagtccttgcacactttgttcggtgacaagttgtgtactgttgctactttgagagaaacttacggtgagatggctgactgttgtgctaagcaagagccagaaagaaacgagtgtttcttgcagcacaaggacgacaacccaaacttgccaagattggttagaccagaggttgacgttatgtgtactgctttccacgacaacgaagagactttcttgaagaagtacttgtacgagatcgctagaagacacccatacttctacgctccagagttgttgttctttgctaagagatacaaggctgctttcactgagtgttgtcaggctgctgacaaagctgcttgtttgttgccaaagttggacgagttgagagatgagggtaaggcttcttctgctaagcagagattgaagtgtgcttccttgcagaagttcggagagagagcttttaaggcttgggctgttgctagattgtcccagagatttccaaaggctgaattcgctgaggtttccaaattggttacagacttgactaaggttcacacagaatgttgtcacggtgacttgttggaatgtgctgatgacagagctgacttggctaagtacatctgtgaaaaccaggactccatctcctccaagttgaaagaatgttgtgagaagcctttgttggagaagtcccactgtattgctgaggttgagaacgacgaaatgcctgctgatttgccttccttggctgctgacttcgttgaatccaaggacgtttgtaagaactacgctgaggctaaggatgttttcttgggtatgttcttgtatgaatatgctagaagacatccagactactccgttgttttgttgttgagattggctaaaacttacgagactactttggaaaagtgttgtgctgctgctgacccacacgaatgttacgctaaggttttcgacgagttcaagccattggttgaggaaccacagaacttgattaagcaaaactgtgagttgttcgagcagttgggtgagtacaagttccagaacgctttgttggttagatacactaagaaggttccacaggtttccactcctactttggttgaagtttccagaaacttgggtaaggttggttccaagtgttgtaagcacccagaggctaagagaatgccatgtgctgaagattacttgtctgttgttttgaaccagttgtgtgttttgcacgaaaagactccagtttccgacagagttacaaaatgttgtactgagtccttggttaacagaagaccttgtttctctgctttggaagttgacgagacttacgttccaaaagagttcaacgctgagactttcactttccacgctgacatctgtactttgtccgagaaagagagacagatcaagaagcagactgctttggttgagttggttaagcacaagccaaaggctacaaaagagcaattgaaggctgttatggacgactttgctgctttcgttgagaaatgttgtaaggctgacgacaaagagacttgtttcgctgaagagggtaagaagttggttgctgcttctcaagctgctttgggtttgtagtaaggatcc

SEQ ID NO: 27 - DNA, 1992 bp, Ogataea minuta
  CDS (1)..(1992)
atggctattcctgacgaattcgatatcatcgttgtgggtggaggctcatgcggctgcgccatcgccggtagactcggtaacctcgacccggacgttactgtggctctcatcgagggtggtgagaacaacatcaataacccatgggtctaccttcctggtgtctatccaagaaacatgagactcgactccaagacggctaccttctacaactcgagaccatccaagcacctgaacggcagaagggccattgtcccctgcgctaacattcttggtggaggttcctccatcaacttcctcatgtacaccagagcctcggcctccgactacgacgactgggagcaagagggatggaccaccgacgagctgcttccgctcatgaagaagctcgagacgtatcaacgtccttgcaacaacagggaggtgcacggtttcgacggtccgatcaaggtctccttcggtaactacacctacccaactgcccaagacttcctgagagcctgcgagtcgcagggtattcctttcaacgacgatcttgaagacctcaaggcctcgcacggagctgagtactggctcaagtggatcaacagggatctcggtagaagatcggactcggcacacgcctacatccaccctaccatgagaaacaagagcaatctgttcctcattacgtccaccaaggctgacaaggtgatcattgagaacggcgttgctgtcggtgtcaggaccgttccaatgaagccggtcgagaccaaaaaccctccaagcaggatcttcaaggccagaaagcaaattgtggtttcgtgcggtacgatctcctctccattggtgctgcaaagatctggtatcggtgcggcccacaagctgagacaagcgggcatcaagccgatcgtcgacttgcctggtgtcggtgagaacttccaggaccactactgcttcttcaccccatactattccaagccagaggttccaacctttgacgactttgtcagaggtgacccagtcgctcaaaagtccgcctttgaccagtggtactccaacaaggacggtcctcttaccaccaacggtatcgaggctggtgtcaagatcagaccaaccgacgaggagttggccacggctgacgatgacttcatccaagggtaccacgagtactttgacaacaagccagacaagccactgatgcattactctgtcatttccggtttcttcggtgaccacaccaagattccaaacggcaagttcttcaccatgttccactttttggagtacccattttcgagaggtttcgtttatgctgtttccccagacccatacgaagctccagactttgatccaggtttcctgaacgattccagagacatgtggcctatggtttggtcttacaagaagtcgagacagacagccagaagaatggagtcgtttgctggtgaagtcacctcgcaccacccactctacccggttgactctccagcccgtgccaaggacttggatctcgagacatgcaaggcatttgctggaccaaaccacttcaccgccaacttgtaccacggttcctggactgttccaattgagaagccaacgccaaagaacgactcgcacgtgacctgcaaccaggtcgagatcttctccgacattgactactctgccgaggacgatgaggctattgtcaagtacatcaaggagcacactgagaccacctggcactgtttgggaacctgttcgatggctccacaagaaggtagcaagatcgctccaaagggtggtgttgtcgatgccagattgaacgtgtacgaagtgaagaacctcaaggttgccgacctgtcgatctgcccagataacgttggatgtaatacttactccactgctcttctgattggtgagaaggctgccactttggtcgccgaggacctgggatactcaggatctgatctcgccatgaccattccaaacttcaagctaggtacttacgaggagaagggtctggctagattctaa

SEQ ID NO: 28 - protein, 663 aa, Ogataea minuta
MAIPDEFDIIVVGGGSCGCAIAGRLGNLDPDVTVALIEGGENNINNPWVYLPGVYPRNMRLDSKTATFYNSRPSKHLNGRRAIVPCANILGGGSSINFLMYTRASASDYDDWEQEGWTTDELLPLMKKLETYQRPCNNREVHGFDGPIKVSFGNYTYPTAQDFLRACESQGIPFNDDLEDLKASHGAEYWLKWINRDLGRRSDSAHAYIHPTMRNKSNLFLITSTKADKVIIENGVAVGVRTVPMKPVETKNPPSRIFKARKQIVVSCGTISSPLVLQRSGIGAAHKLRQAGIKPIVDLPGVGENFQDHYCFFTPYYSKPEVPTFDDFVRGDPVAQKSAFDQWYSNKDGPLTTNGIEAGVKIRPTDEELATADDDFIQGYHEYFDNKPDKPLMHYSVISGFFGDHTKIPNGKFFTMFHFLEYPFSRGFVYAVSPDPYEAPDFDPGFLNDSRDMWPMVWSYKKSRQTARRMESFAGEVTSHHPLYPVDSPARAKDLDLETCKAFAGPNHFTANLYHGSWTVPIEKPTPKNDSHVTCNQVEIFSDIDYSAEDDEAIVKYIKEHTETTWHCLGTCSMAPQEGSKIAPKGGVVDARLNVYEVKNLKVADLSICPDNVGCNTYSTALLIGEKAATLVAEDLGYSGSDLAMTIPNFKLGTYEEKGLARF

SEQ ID NO: 29 - DNA, 798 bp, Ogataea minuta
  CDS (1)..(798)
atgtcctcgactaagacatacgcgcaaagggcggcggctcatccgtcgcctgtggccagaagactgctgaacttgatggaatccaagaagacgaacttgtgtgcctcggtcgatctcacctctacaaaggaccttttggagctgttggacaagctgggaccgttcatttgtctggtcaagacacacatcgacattgtggaagacttttcgtacgaaaacaccgtggtgccgctgctgaaactggccaagaaacacaacttcatgatcttcgaggaccgaaaatttgccgatataggcaacaccgtcaaactccagtacaagggaggagtttaccaaatcgcaaagtgggccgatatcaccaacgcccacggagtgaccggctcgcgaattgtctcgggtctcagacaggctgcccaggagaccaccgacgagccaagaggtctgctcatgctggctgagctgtcgtctgaaggctcgctcgcgtacggagagtacaccaaaaagacggttgaaatcgcaaagtccgacagagattttgtgatcggtttcattgcgcaaaacgacatgggtggccgcgatgagggcttcgactggctcatcatgaccccaggtgtcggactcgacgacaccggtgacgctctgggccagcagtaccgcacggtcagcgccgttatgaagacgggaactgacatcataatcgtgggcaggggactgttcggcaagggaagagaccctgtcgtggaaggcgaaagatacagaaaggctggatgggacgcttatttgagtcgtgtcgcatga

SEQ ID NO: 30 - protein, 265 aa, Ogataea minuta
MSSTKTYAQRAAAHPSPVARRLLNLMESKKTNLCASVDLTSTKDLLELLDKLGPFICLVKTHIDIVEDFSYENTVVPLLKLAKKHNFMIFEDRKFADIGNTVKLQYKGGVYQIAKWADITNAHGVTGSRIVSGLRQAAQETTDEPRGLLMLAELSSEGSLAYGEYTKKTVEIAKSDRDFVIGFIAQNDMGGRDEGFDWLIMTPGVGLDDTGDALGQQYRTVSAVMKTGTDIIIVGRGLFGKGRDPVVEGERYRKAGWDAYLSRVA

SEQ ID NO: 31 - DNA, 1620 bp, Ogataea minuta
  CDS (1)..(1620)
atgaagttatcccagtctgctgcggtggctattctgtcttcgttggcagcagtggaggccttggtcatcccgttatttgacgacttgccagcagagtttgcccttgttccaatggatgcgaaagcggaagtcatttctgacgttcctgtcgactcggccattagtgatgctcctatcgcggcactaaatgatgctccaagccctctcgtcacatcgctgatcgcatctcaaaatttgattccaaactcttatattgtcgttttcaagaatggcctagcttccggggcagttgacttccacatggagtggctcaaggaaacgcactcccaaaccctggctgctttgtctaaggacatgccagcagaagaattggccgccgaaggtttcgtttccgaaagcattgatcttactgaggtgtttagcatctccgatttgttcagtggatataccggatacttcccggagaaggtggttgacctcatcagaagacaccctgacgtggcgttcgttgagcaggactcgagagttttcgccgataagtcgtctactcaaaacggtgctccttggggtttgtctagaatctctcacagagagcctctcagtctcggcaatttcaacgagtacgtttacgacgatcttgctggagatggcgtcacggcttatgtcattgataccggtatcaatgtgaagcacgagcagttcggtggcagagcagagtggggtaagaccatcccaaccggtgatgatgatattgacggaaacggtcacggtactcactgcgctggtacaattggctcggaagattatggagtttctaagaactccaaaattgtcgcagtgaaggttttgagatctaacggttctggttccatgtctgacgtgatcaagggtgttgaattcgctgcaaatgatcacgttgccaagtctaaagccaagaaggacggtttcaagggatcgactgccaacatgtctttgggaggtggcaagtctcctgctcttgacttggctgtcaatgccgctgtcaaagctggtttacactttgctgttgccgctggtaacgacaatgctgacgcatgcaactattctcctgctgctgcagagaacgcagtcactgttggtgcgtccactttgtctgactctagagcttacttttccaactatggtaaatgtgttgacatttttgctccgggcttgaacatcctttccacctacataggttctgacactgccaccgccactctttctggtacatcgatggcctcccctcacgtttgtggtctgttgacctactttttgagcttgcaaccagaatcgtcgtcgttgttttcttcggcagctatctcccctgctcagctgaagaagaacctgatcaagtttggtacgaagaacgttttgtctgagattccatcggacggaaccccaaatattctcatttacaacggtgctggcaagaacatcagtgacttctgggcgtttgaagacgaggcctcggccaagtccgacttgaagaaggctgtcgatattgccacaagtgttgacttagacctgcaagatatcaaggagaagttcaaccatattttggaggaggtcgccgaagaggttgctgatttgttcgattag

SEQ ID NO: 32 - protein, 539 aa, Ogataea minuta
MKLSQSAAVAILSSLAAVEALVIPLFDDLPAEFALVPMDAKAEVISDVPVDSAISDAPIAALNDAPSPLVTSLIASQNLIPNSYIVVFKNGLASGAVDFHMEWLKETHSQTLAALSKDMPAEELAAEGFVSESIDLTEVFSISDLFSGYTGYFPEKVVDLIRRHPDVAFVEQDSRVFADKSSTQNGAPWGLSRISHREPLSLGNFNEYVYDDLAGDGVTAYVIDTGINVKHEQFGGRAEWGKTIPTGDDDIDGNGHGTHCAGTIGSEDYGVSKNSKIVAVKVLRSNGSGSMSDVIKGVEFAANDHVAKSKAKKDGFKGSTANMSLGGGKSPALDLAVNAAVKAGLHFAVAAGNDNADACNYSPAAAENAVTVGASTLSDSRAYFSNYGKCVDIFAPGLNILSTYIGSDTATATLSGTSMASPHVCGLLTYFLSLQPESSSLFSSAAISPAQLKKNLIKFGTKNVLSEIPSDGTPNILIYNGAGKNISDFWAFEDEASAKSDLKKAVDIATSVDLDLQDIKEKFNHILEEVAEEVADLFD

SEQ ID NO: 33 - protein, 814 aa, Saccharomyces cerevisiae
MKVRKYITLCFWWAFSTSALVSSQQIPLKDHTSRQYFAVESNETLSRLEEMHPNWKYEHDVRGLPNHYVFSKELLKLGKRSSLEELQGDNNDHILSVHDLFPRNDLFKRLPVPAPPMDSSLLPVKEAEDKLSINDPLFERQWHLVNPSFPGSDINVLDLWYNNITGAGVVAAIVDDGLDYENEDLKDNFCAEGSWDFNDNTNLPKPRLSDDYHGTRCAGEIAAKKGNNFCGVGVGYNAKISGIRILSGDITTEDEAASLIYGLDVNDIYSCSWGPADDGRHLQGPSDLVKKALVKGVTEGRDSKGAIYVFASGNGGTRGDNCNYDGYTNSIYSITIGAIDHKDLHPPYSEGCSAVMAVTYSSGSGEYIHSSDINGRCSNSHGGTSAAAPLAAGVYTLLLEANPNLTWRDVQYLSILSAVGLEKNADGDWRDSAMGKKYSHRYGFGKIDAHKLIEMSKTWENVNAQTWFYLPTLYVSQSTNSTEETLESVITISEKSLQDANFKRIEHVTVTVDIDTEIRGTTTVDLISPAGIISNLGVVRPRDVSSEGFKDWTFMSVAHWGENGVGDWKIKVKTTENGHRIDFHSWRLKLFGESIDSSKTETFVFGNDKEEVEPAATESTVSQYSASSTSISISATSTSSISIGVETSAIPQTTTASTDPDSDPNTPKKLSSPRQAMHYFLTIFLIGATFLVLYFMFFMKSRRRIRRSRAETYEFDIIDTDSEYDSTLDNGTSGITEPEEVEDFDFDLSDEDHLASLSSSENGDAEHTIDSVLTNENPFSDPIKQKFPNDANAESASNKLQELQPDVPPSSGRS

SEQ ID NO: 34 - protein, 609 aa, Homo sapiens [sequence omitted]

SEQ ID NO: 35 - DNA, 1551 bp, Ogataea minuta
  CDS (1)..(1551)
atgaagttatttggattgactacattgaccagcatcctggccgccctcacagtggtgagcgccaccgaagagccagcagttgcctcgccagactcggcggtggtgaagctgacggccgacactttcgagagcttcatcaaggagaacccattggttctggcggagttttttgcgccatggtgtggccactgcaagaagctgggtccagaattcagcgcagccgcagaccagctggtggagaagaacatcaagcttgcacagatcgactgtaccgaggaaagagatctgtgttcgtcgcacggaatcagaggatacccaactttgaaggtgttcaggggcgctagtgagcctgctgactaccaaggcgccagagaacaggaagctattgtcagtcaaatgatcaagctttctttacctgctgtttccgtcattgaggattctgccgacctgtttgataccattgcagaagtctccgacgccctcattgtgcaagtgtttcctgcgggagctgctcagtcttccaacgagacgttctacgaagtcgccaacgaactgagaaacgactttgtttttgtctccaccactaacgaggggtacgtgaaaaagtacgcgaaggactcaaagtcacctgcttatgtcatcttcaggcaaggagaaaaggttgaagatgcgtccacatacaccggaaagactgttgacgacactcacttgaagcagttcatcaataccgaaaccaaacctctgtttggtgaaatcaccggcaacactttcaagacctacatggaggccgagcttcctttggcgtactttttctgggacgaagagtctcaaagggccgaggtcgctgacatcatcaccgagctggccaagaagtttagaggagagatgaactttgttggtttggaagccaagagatacggtatgcatgccaagaacctcaacatggaggaaaagttccccttgttcgccatccacgatttgaccggaaacctcaagtacggtatttctcaagagtctgatcttgacgtcaaggaaatccctaagttcgttgaggatttcaagaagggcaagttgcaagcaattgtcaagtctgagccaattccagaagtccaagaggagtccgtgtaccacctggttggacacgagcacgacgccatcaccaagcagaagaaggacgttttggttgagtactacgccccatggtgtggacactgcaagaagctggctccaacttacgaaattttggccagcatctaccagaacgacactgatgccaaggaaaaggttgtgattgccaagattgaccacactgccaacgatgttgccggtgtcgacatcgccggttatccaaccatcatcttgtatcctggtgacgaatctgagccggttgtgtacgagggttctagaactctagaggctctcagttcattcatcaaggagaagggttcgaacggcgttgacgctttgtccatcaaggaatcgcgtgttgaaaaagaagccgatgctcaagccgatgctcctgacgctggcgtggctcacgacgagttgtaa

SEQ ID NO: 36 - protein, 516 aa, Ogataea minuta
MKLFGLTTLTSILAALTVVSATEEPAVASPDSAVVKLTADTFESFIKENPLVLAEFFAPWCGHCKKLGPEFSAAADQLVEKNIKLAQIDCTEERDLCSSHGIRGYPTLKVFRGASEPADYQGAREQEAIVSQMIKLSLPAVSVIEDSADLFDTIAEVSDALIVQVFPAGAAQSSNETFYEVANELRNDFVFVSTTNEGYVKKYAKDSKSPAYVIFRQGEKVEDASTYTGKTVDDTHLKQFINTETKPLFGEITGNTFKTYMEAELPLAYFFWDEESQRAEVADIITELAKKFRGEMNFVGLEAKRYGMHAKNLNMEEKFPLFAIHDLTGNLKYGISQESDLDVKEIPKFVEDFKKGKLQAIVKSEPIPEVQEESVYHLVGHEHDAITKQKKDVLVEYYAPWCGHCKKLAPTYEILASIYQNDTDAKEKVVIAKIDHTANDVAGVDIAGYPTIILYPGDESEPVVYEGSRTLEALSSFIKEKGSNGVDALSIKESRVEKEADAQADAPDAGVAHDEL

SEQ ID NO: 37 - DNA, 936 bp, Ogataea minuta
  CDS (1)..(936)
atgaaagtggcaagtttgatagcattggtcgttacgccgattattgctgccactggggtggtagcagatccgcagcagcaggccaaaagacctggtttttacaagaattcaaagcatatctacaatctcactccccagaactttgacgacgtggtcctgcaaaccaaccatacgtctgtcgtggagttctatgcgccatggtgtggctattgcgcagagtttgagagccagtaccgcaaagcagcaaagatcggatcggagttcgtgaattttgcggccgttaactgcgacgaagacaagaacaaaccattgtgcaacaagtaccgcgtcgaagggttcccgacggtgatggttttccgtccagcgaaggtcaactcggcgggatccaacggcaacaggcctcattcctccgaaacgtatcggggcgagagaacggcggctaagttgctcgagcatgtcaagggccgtgtggtgaattacgtgaagagaatcaagctcaacaaacttgatgaatttctcaaaccgaatgaaaagagcagagtcttgctggtgacttcaaaaagcactctttcgccggttttcaagagcctgtcgatcgattttctcgactcagtcacgttggcatacctcactctgagcgaaaacgactccgaaggtagagacaaactgctggaaaagattcctgccctcaaagcggacttcaaagtcccgactttactcgccatcgacaagggaacgaaaaatgtgacggtttatgattccgaatcgatgtcgaaaaaagagctgacgaagttcatgtctaagttcggccagccacaagagggggcaatgagcgaaagagggggcatcttgaaaggaatcaagaagggtgcttacaagagcttcaaagattacaaaaagaagatgcaacaagctcttgaaaaagatgagctatga

SEQ ID NO: 38 - protein, 311 aa, Ogataea minuta
MKVASLIALVVTPIIAATGVVADPQQQAKRPGFYKNSKHIYNLTPQNFDDVVLQTNHTSVVEFYAPWCGYCAEFESQYRKAAKIGSEFVNFAAVNCDEDKNKPLCNKYRVEGFPTVMVFRPAKVNSAGSNGNRPHSSETYRGERTAAKLLEHVKGRVVNYVKRIKLNKLDEFLKPNEKSRVLLVTSKSTLSPVFKSLSIDFLDSVTLAYLTLSENDSEGRDKLLEKIPALKADFKVPTLLAIDKGTKNVTVYDSESMSKKELTKFMSKFGQPQEGAMSERGGILKGIKKGAYKSFKDYKKKMQQALEKDEL

SEQ ID NO: 39 - DNA, 930 bp, Ogataea minuta
  CDS (1)..(930)
atgtttatggagatcggagaggcgtacgaggtgctgatggattcagaaaagagatccatatatgacaggtacggagaggaaggcttgaaaggcggtgcagggggcggaggaggaggacaacaccacgatccgttcgacatgtttgccaactttttcggcggcgccggtgggcgtcaacaagcacggggagttccaagagggtcggacattgccacggagatggagtttaccttgaaagagttttacaacggagtgaatagcgacttttcactcgaactgcaagacatctgtgaccgttgtgacggaagcgggtcgcaggacgggaaagtgcacaagtgttctcgatgcaatggtcgtggccgggtgttagtgaagagacagttgggtcctggcatgttccagcagatggagtcggcgtgtcccgactgtcgtggagcaggaaaacagattactcaccattgcaagaagtgtcggggtggcggggttgtccgtggaattcgcaacttcaacatccaccttgagccaggaactccgcgcgaccacgtcgaagtgtacgagggtcagtccgacaggtctccggagtgggaggctggtaacttacgtctgagcgtcagagagaagaaaagcggaaaccttgggtatcgtcggatcggaaacaatctgtaccgcacagagatcttgacgctgagcgagtctctgaagggtggttgggtccgcgagatcccgtttctggacaactacgacgccgtcttaaagctggaaagaccactcggaagtgttgttaccagcggggaagtgcaggtggtgaaaggaaagggtatgccgattgccaactccgtggatcagtttggcgatctgtatgtggagtatgtggtgttgtatccgggaggaagcccgaagaaggtgagcaagttgcacgacgagctgtga

SEQ ID NO: 40 - protein, 309 aa, Ogataea minuta
MFMEIGEAYEVLMDSEKRSIYDRYGEEGLKGGAGGGGGGQHHDPFDMFANFFGGAGGRQQARGVPRGSDIATEMEFTLKEFYNGVNSDFSLELQDICDRCDGSGSQDGKVHKCSRCNGRGRVLVKRQLGPGMFQQMESACPDCRGAGKQITHHCKKCRGGGVVRGIRNFNIHLEPGTPRDHVEVYEGQSDRSPEWEAGNLRLSVREKKSGNLGYRRIGNNLYRTEILTLSESLKGGWVREIPFLDNYDAVLKLERPLGSVVTSGEVQVVKGKGMPIANSVDQFGDLYVEYVVLYPGGSPKKVSKLHDEL

SEQ ID NO: 41 - DNA, 1137 bp, Ogataea minuta
  CDS (1)..(1137)
atgaaagtcacgtctatctggcaagtcctgtttatgttgatcgccggcgttatggcgtcagcgtccaaagtgatggagctgaacgacaagaactttgacgaggtggttctcaactccggaaagacctcgctagtggaattctacgcgtcgtggtgcagtcactgcaagaagttggagcctacttgggaagagctggcctcggcgtacggaaacaagaacgatatccagatcgtcaagatcgacgctgacgaaaacggaaacgtgggaagaaaattcggaatcaagggatttcccacgctgaaactgttcaaaaaagatgatctcaacaacccagtggaatttgaaggctccagggacttccattctttcaccaacttcattgctgcacacacgggtatcaaggctgccaacgcggttcccactgagccgtccaaagtggtggaactgcacgatggaaatttggaggagcttgttaaggagcagggaaaaaatgctctttttgcaatcaccgcagagtggtgtggttactgcaagaagctcaagcctacatgggagcagctggctgccgttttccaaggcgacgaggaaaacatcttgattggacaggtccaaaccaccggcgataacccaacagaatggatccaggagaaatacaacctccagtcgttccccacaatagtcttcatcgagaagggcaacctggacgagcctgtgttctatccgtacggaagagagctgggagacctggttgagttcgttaacacccaggccggaactcaccggaacgagaaaggcgagctggactccgaggccgggctgatacacgcagtcgacgagctggttgagcagtttgtcggttcctcgagcagcggcagaaaaaatctggttccgaaattcttggaagctttgaaatcggctgacaccgacaatgcattgtcgaaagaagtgaaatactacaacaagatcatccatacgatggtcaacggtccctttgacttcgtcgcgaaagaaaccgctagactggagtcgctactgaagtcggatctgtcttcccgagccagagactcagcttcctttagactcaacatcctcaagtttttcagcgatcctgcccctccagccaaggatgagctgtga

SEQ ID NO: 42 - protein, 378 aa, Ogataea minuta
MKVTSIWQVLFMLIAGVMASASKVMELNDKNFDEVVLNSGKTSLVEFYASWCSHCKKLEPTWEELASAYGNKNDIQIVKIDADENGNVGRKFGIKGFPTLKLFKKDDLNNPVEFEGSRDFHSFTNFIAAHTGIKAANAVPTEPSKVVELHDGNLEELVKEQGKNALFAITAEWCGYCKKLKPTWEQLAAVFQGDEENILIGQVQTTGDNPTEWIQEKYNLQSFPTIVFIEKGNLDEPVFYPYGRELGDLVEFVNTQAGTHRNEKGELDSEAGLIHAVDELVEQFVGSSSSGRKNLVPKFLEALKSADTDNALSKEVKYYNKIIHTMVNGPFDFVAKETARLESLLKSDLSSRARDSASFRLNILKFFSDPAPPAKDEL

SEQ ID NO: 43 - DNA, 1728 bp, Ogataea minuta
  CDS (1)..(1728)
atgaagcacgtgataagtggccgatcccgggtggcgttgggctggattctgacgtggttgatctgtgcgatttgcgctgttcccggagctgggttgcaggagatttcgtccttggagaggaaaccggcttacctgtcgccgcagtatgagtacgacaacattcacgagtttgagtcgacgcctttccgcgattacgagacgttcacggggtccaaggtgagtgagagttcgaacgtgacttttggccagatcaacgcgctcaacaacgaaatcagaccggttttgcacgatctgattaacgagaacttcttcaaaatctttcgactcaacctgtacaaggagtgtccgttctggtcgagttcggagggattttgcatgcacaagagctgtgccgtggacaccattgacgactggaaagatcttccggagatatggcagcccgaggctctgggtcggatcgagtcgttgacgcgagaaccgcctacgacgatatctgacgcgggaaatggctcgtgtgtcgctgcaggcggacggagcacgcgggattactgcgaactggacgaggtcaacgaggactcggtatacgtgaatctggtggacaatcccgagagattcacggggtacggaggagatcagtcgttccaaatttggcgcagcatttacaacgagaactgtttcaatttgggctcggatcaatgtctcgagaagaactttttctacaagttgatcagtggaatgcactcgtcgatctcgactcatctgaccaacgagtacctgaacttcaagaccaagcagtatggacaggatctcaagcagttcatgatccgggtgggggacttccctgaccggttcgagaacttgtatctgaactacgttctggtggtgaagtccttgatcaagctggagcagtcgggtgtgctggacaacctccagttctgtgacgaggaggtgtttcagacgaaggagaaggagctaaaacgcgagctgaaggagatgatttctccgttctaccagctggcggaaggtgggaaagtggacgagtgtctgttcaacgagcactcgctgttccagagcgaggactcgacgtatctgaaggacgagttcagtgagaacttcaggaacgtgtcgcggatcatggattgtgtccactgcgacaggtgcagactgtgggggaaggtgcagaccaccgggtacggaactgcgctgaagattttgtttgagctggatgcaagcgacagccacgagctgggcaagaatttccagatctccaaaatcgagctggtcgccctgatcaacacgtttgacaggctgtccaagagcgtgcacgccatcggaaacttcaaacaactgtacgatctgagaatgaaacaggaggaagaaggggggtccatgattactgcagacacgtttgacttggagcaattgctgttgacagaccagacggtggacgtattcggccagagcacttctgagccagaaaccccgtcggacgttagatatcccgacagaacacggggctcgctcgttcccgaggggctcggcgaggcgttcaagacagagctgtacagcgtttatcaggcgttctactttgtcgtgaccagctacaccatgttccccaagctgatctacaactacctgctgatccgggtggtgtactggtggaacatctttgtgggtcatgtacacgaggactttgacgtggatcgcctgtatcgtttggagctataa

SEQ ID NO: 44 - protein, 575 aa, Ogataea minuta
MKHVISGRSRVALGWILTWLICAICAVPGAGLQEISSLERKPAYLSPQYEYDNIHEFESTPFRDYETFTGSKVSESSNVTFGQINALNNEIRPVLHDLINENFFKIFRLNLYKECPFWSSSEGFCMHKSCAVDTIDDWKDLPEIWQPEALGRIESLTREPPTTISDAGNGSCVAAGGRSTRDYCELDEVNEDSVYVNLVDNPERFTGYGGDQSFQIWRSIYNENCFNLGSDQCLEKNFFYKLISGMHSSISTHLTNEYLNFKTKQYGQDLKQFMIRVGDFPDRFENLYLNYVLVVKSLIKLEQSGVLDNLQFCDEEVFQTKEKELKRELKEMISPFYQLAEGGKVDECLFNEHSLFQSEDSTYLKDEFSENFRNVSRIMDCVHCDRCRLWGKVQTTGYGTALKILFELDASDSHELGKNFQISKIELVALINTFDRLSKSVHAIGNFKQLYDLRMKQEEEGGSMITADTFDLEQLLLTDQTVDVFGQSTSEPETPSDVRYPDRTRGSLVPEGLGEAFKTELYSVYQAFYFVVTSYTMFPKLIYNYLLIRVVYWWNIFVGHVHEDFDVDRLYRLEL

SEQ ID NO: 45 - DNA, 2700 bp, Ogataea minuta
  CDS (1)..(2700)
atggattctacgcaatttacagacagagctctagacatagtgacgacggctcagaagctatgtcagcagaactcaaacgcacagatagtgccattgcactttctggcggcaatgactccgacctccagtgagggggaggcaatctatctcaagacgttgattgagagaggtcgatttgactggactgcttttgaaagagccgttaataaagcagtggtgcggctcccaagcgtggccggttccaacaccgagccaagcatttccgcctccgccgccagcattatcaccaacgcatccaagatcaaggcacagcagaaagactcgtacatcggccaagaccacattctctcggcccttttggacgattctagtatccaggctgtgctgaaggaagcgaacgtcaagcccgacgcattgaagacacagatcgttgaactcagaggcaatcagagaattgattctcgtcaagctgattcgtctcagaagtttgagtttctgtccaagtacgcccttgatcttaccgagcaggcgttgcagggtaagattgaccctgtgataggcagagaggaggagatcagaagggccattcgggttctttcgagacgggccaagtcgaacccgtgtctgattggagatcctggtgttggtaagactagtattgttgagggagtcgcacagaggatagtggacaacgatgttcctaccgttttacaggggtgcaagctgtactcgctggacttgggtgcgttgaaagcgggagccaagtaccagggtgagttcgaggaaagattgaagggagttttgagcgacatcgaaagctcgaactcgatgatcatcttgttcatcgacgagatccacatgttgatgggcgatggaaagtcggacgctgccaacttgctgaagcctgctctggcgagaggtcagttccactgcatcggtgcgactaccgtgaccgagtacagaaagcacatcgagaaggacggtgctttcgagagaagatttcagagaatcgacgtgagagagccgaccatcagagaaaccgttgccattctcagaggactgcagccaaggtacgagatccaccacggtgtccgaatcctggacagtgccctggtgacggctgctcaactggcctccagatacctcacctacagaaagcttccggactctgcagtggatctgattgacgagtccgccgcgggagtcgctgttgccagggactccaagccggaggagctggactccaaggagagacagctacagctgatcgaggttgagatcaatgctctggaaagagaccaggacgccgacacgtccaccaaggagagactggagcaggccagacagagaagacagaacctggaggaagagctggctccattgagggagaagtaccagcaggaaagagcgggccacgaggagctgactgctgccaagagaaagttagacgacctcgaagttaaggcgcaagatgcggagagaagacacgactctcagaccattgcggacctgcggatgtttgccattccggacgtgaaacgcagaattgaggagttggaacagaaagtggttgaagaagaggccacttctgaagatttcatggtgaagaacgttgttggttcggagcaagtttccgagaccgcggccaggttgaccggtattccggtcagcaagctgacgcaggcagaaaatgctaagctgatcaccatggagaaggagctgtccgctgccgttgtcggacagggcgaggctgtcaaggctgtttcgaactcgatcagattgtcgagatctggtctggccaatcctaaccagccggcctcgttcctgttcctgggactctcgggttccggtaagacggagctggccaaaaagctggcagggttcctgttctcggatgagaaggccatgatcagaatcgactgttcagaactgatggagaagtactcggtttccaagctgctgggctccaccgccggttacgttggctacgaggagggtggtatgttgaccaaccagcttttgagaagaccgtactctgtggtgttgttcgacgaggtcgagaaggccgctcccgaggtgctgaacattctcctgcagatgttggacgacggaagaatcaccgccgccaacggtactctcgtcaactgctcgaacgctatcgtgatcatgacgtccaacctcggcgcggagtacatcaacgcatcaaagggaacgaaggtgaccgacgaggtcagaagcctggtgatgaacactgtcaagggacatttcagaccggagttcctcaacagaatctcggccaccgtggtgttcaacagactctccagacatgccatcgccaagatcgtgcggctgagactgaaggaaatcgaagagaggttcgaggccaacggcaagtcgatcaaactgaacgtggacgatggggcactcgagtacctgtgcaaaaagggatactctcccgacttgggcgctagaccgctgaacagactgatccagagtgaaatcctgaaccatctggcggtgatggtgttgaacggacaggtgttggacaaagaggaagttcagattacgacgggcagcaagggactgtctgtggttcctaaccatgacatcgaggacgaagctatggacgtggacgtggacgagtggaccgacgccgccgacgacgacgactcgggctacggcagtcctgatctcgattaa

SEQ ID NO: 46 - protein, 899 aa, Ogataea minuta
MDSTQFTDRALDIVTTAQKLCQQNSNAQIVPLHFLAAMTPTSSEGEAIYLKTLIERGRFDWTAFERAVNKAVVRLPSVAGSNTEPSISASAASIITNASKIKAQQKDSYIGQDHILSALLDDSSIQAVLKEANVKPDALKTQIVELRGNQRIDSRQADSSQKFEFLSKYALDLTEQALQGKIDPVIGREEEIRRAIRVLSRRAKSNPCLIGDPGVGKTSIVEGVAQRIVDNDVPTVLQGCKLYSLDLGALKAGAKYQGEFEERLKGVLSDIESSNSMIILFIDEIHMLMGDGKSDAANLLKPALARGQFHCIGATTVTEYRKHIEKDGAFERRFQRIDVREPTIRETVAILRGLQPRYEIHHGVRILDSALVTAAQLASRYLTYRKLPDSAVDLIDESAAGVAVARDSKPEELDSKERQLQLIEVEINALERDQDADTSTKERLEQARQRRQNLEEELAPLREKYQQERAGHEELTAAKRKLDDLEVKAQDAERRHDSQTIADLRMFAIPDVKRRIEELEQKVVEEEATSEDFMVKNVVGSEQVSETAARLTGIPVSKLTQAENAKLITMEKELSAAVVGQGEAVKAVSNSIRLSRSGLANPNQPASFLFLGLSGSGKTELAKKLAGFLFSDEKAMIRIDCSELMEKYSVSKLLGSTAGYVGYEEGGMLTNQLLRRPYSVVLFDEVEKAAPEVLNILLQMLDDGRITAANGTLVNCSNAIVIMTSNLGAEYINASKGTKVTDEVRSLVMNTVKGHFRPEFLNRISATVVFNRLSRHAIAKIVRLRLKEIEERFEANGKSIKLNVDDGALEYLCKKGYSPDLGARPLNRLIQSEILNHLAVMVLNGQVLDKEEVQITTGSKGLSVVPNHDIEDEAMDVDVDEWTDAADDDDSGYGSPDLD

SEQ ID NO: 47 - DNA, 1998 bp, Ogataea minuta
  CDS (1)..(1998)
atgtttaagttcaaccgctctgtgctatctatagctacgatactgtatgcagtgctgttggtagtcctacctttggcttcacaacaattcgtggaagcagaggcgaacgacaactatggtactgttatcggtatcgatttgggaaccacttactcatgtgtgggtgtgatgaaagctggtagagttgagatccttgccaatgaccagggtaacagaattactccatcttatgtggcatttactgatgaagagagacttgtcggagatgccgcaaagaaccagattgcctccaacccaagcaacacaatcttcgatatcaagagactcataggacacagatttgacgataaggttgtgcaaaaagagattgcacacctcccttacaagatcagaaaccaagagggcagaccggtcgttgaggccactgtcaatggagaggtgaccacgttcacggccgaagaagtttcggccatgatcttgggaaagatgaagcaaattgctgaagattatctcggaaagaaggttacccatgctgttgtcacggttcctgcatactttaacgacgcccaaagacaggccactaaggatgctggtaccattgccggtctggaagttttgagaattgttaacgagcctactgccgctgcaattgcttacggtctcgacaagacggacgaagagaagcatatcattgtttacgatttgggtggaggaacttttgatgtttctctgttgacaattgcaggtggagctttcgaggttcgtgccaccgctggtgatacccatcttggtggtgaggactttgattacagagttgtcagacatttcatcaaggtgtttaagaagaagcatggcattgatatcagtgataatccaaaggctcttgctaaattgaagagagaagttgaaaaagctaagagaaccttgtcttctcaaatgtccaccagaattgagattgactcgttcgctgacggtattgacttctccgagtccttatccagggccaagttcgaggaattgaacattgagttgttcaaaaagaccttgaagcctgttcaacgtgttcttgaagacgccaaattcaaggtttcagaaattgatgacattgtcttggttggtggttccacgagaattccaaaggtgcaagagttgctggaaagttacttcaacggcaagcaagtgtccaagggaattaacccagatgaagctgttgcttacggtgcggctgttcaagctggtgtcctctctggtgaagaaggcgttgaagacattgttttgattgatgtgaatccattaactttgggtatcgagacctccggcggtgtcatgaccactttgattaagagaaacactgcaattccaaccaagaagtctcaaattttctctactgctgctgacaatcaacctgttgtcttgatccaagtctatgaaggtgagagagccatggcaaaggataacaatttgctaggaaagttcgagttgaaggatattcctccagccccaagaggtaccccacaaattgaggtgactttcactctggactccaacggaatcctgaaggttgctgccactgataaaggtactggtaagtctaactctatcacaatcacaaacgacaagggtagactttcgaaggaggagattgagaagaaggttgaggaggccgaacagtatgctcaacaagataaggaggtcagagagaagatcgagagcagaaacggacttgagaactacgcccactcgttgaaaaaccaagtgaacgatgagaccggattcggctccaagcttgatgaggatgacaaggaaactttgttggatgccatcaacgaggcattggagtacttggacgacaactttgagaccgcaacaaagcaagactttgaggatcagaaggaaaaattgagtaaggttgcttacccaattacttcaaagttgtatgatacgccacctactagtgacgaagatgatgaggatgactgggatcatgatgagctgtga

SEQ ID NO: 48 - protein, 665 aa, Ogataea minuta
MFKFNRSVLSIATILYAVLLVVLPLASQQFVEAEANDNYGTVIGIDLGTTYSCVGVMKAGRVEILANDQGNRITPSYVAFTDEERLVGDAAKNQIASNPSNTIFDIKRLIGHRFDDKVVQKEIAHLPYKIRNQEGRPVVEATVNGEVTTFTAEEVSAMILGKMKQIAEDYLGKKVTHAVVTVPAYFNDAQRQATKDAGTIAGLEVLRIVNEPTAAAIAYGLDKTDEEKHIIVYDLGGGTFDVSLLTIAGGAFEVRATAGDTHLGGEDFDYRVVRHFIKVFKKKHGIDISDNPKALAKLKREVEKAKRTLSSQMSTRIEIDSFADGIDFSESLSRAKFEELNIELFKKTLKPVQRVLEDAKFKVSEIDDIVLVGGSTRIPKVQELLESYFNGKQVSKGINPDEAVAYGAAVQAGVLSGEEGVEDIVLIDVNPLTLGIETSGGVMTTLIKRNTAIPTKKSQIFSTAADNQPVVLIQVYEGERAMAKDNNLLGKFELKDIPPAPRGTPQIEVTFTLDSNGILKVAATDKGTGKSNSITITNDKGRLSKEEIEKKVEEAEQYAQQDKEVREKIESRNGLENYAHSLKNQVNDETGFGSKLDEDDKETLLDAINEALEYLDDNFETATKQDFEDQKEKLSKVAYPITSKLYDTPPTSDEDDEDDWDHDEL

SEQ ID NO: 49 - DNA, 1569 bp, Saccharomyces cerevisiae
  CDS (1)..(1569)
atgaagttttctgctggtgccgtcctgtcatggtcctccctgctgctcgcctcctctgttttcgcccaacaagaggctgtggcccctgaagactccgctgtcgttaagttggccaccgactccttcaatgagtacattcagtcgcacgacttggtgcttgcggagttttttgctccatggtgtggccactgtaagaacatggctcctgaatacgttaaagccgccgagactttagttgagaaaaacattaccttggcccagatcgactgtactgaaaaccaggatctgtgtatggaacacaacattccagggttcccaagcttgaagattttcaaaaacagcgatgttaacaactcgatcgattacgagggacctagaactgccgaggccattgtccaattcatgatcaagcaaagccaaccggctgtcgccgttgttgctgatctaccagcttaccttgctaacgagacttttgtcactccagttatcgtccaatccggtaagattgacgccgacttcaacgccaccttttactccatggccaacaaacacttcaacgactacgactttgtctccgctgaaaacgcagacgatgatttcaagctttctatttacttgccctccgccatggacgagcctgtagtatacaacggtaagaaagccgatatcgctgacgctgatgtttttgaaaaatggttgcaagtggaagccttgccctactttggtgaaatcgacggttccgttttcgcccaatacgtcgaaagcggtttgcctttgggttacttattctacaatgacgaggaagaattggaagaatacaagcctctctttaccgagttggccaaaaagaacagaggtctaatgaactttgttagcatcgatgccagaaaattcggcagacacgccggcaacttgaacatgaaggaacaattccctctatttgccatccacgacatgactgaagacttgaagtacggtttgcctcaactctctgaagaggcgtttgacgaattgagcgacaagatcgtgttggagtctaaggctattgaatctttggttaaggacttcttgaaaggtgatgcctccccaatcgtgaagtcccaagagatcttcgagaaccaagattcctctgtcttccaattggtcggtaagaaccatgacgaaatcgtcaacgacccaaagaaggacgttcttgttttgtactatgccccatggtgtggtcactgtaagagattggccccaacttaccaagaactagctgatacctacgccaacgccacatccgacgttttgattgctaaactagaccacactgaaaacgatgtcagaggcgtcgtaattgaaggttacccaacaatcgtcttatacccaggtggtaagaagtccgaatctgttgtgtaccaaggttcaagatccttggactctttattcgacttcatcaaggaaaacggtcacttcgacgtcgacggtaaggccttgtacgaagaagcccaggaaaaagctgctgaggaagccgatgctgacgctgaattggctgacgaagaagatgccattcacgatgaattgtaa

SEQ ID NO: 50 - protein, 522 aa, Saccharomyces cerevisiae
MKFSAGAVLSWSSLLLASSVFAQQEAVAPEDSAVVKLATDSFNEYIQSHDLVLAEFFAPWCGHCKNMAPEYVKAAETLVEKNITLAQIDCTENQDLCMEHNIPGFPSLKIFKNSDVNNSIDYEGPRTAEAIVQFMIKQSQPAVAVVADLPAYLANETFVTPVIVQSGKIDADFNATFYSMANKHFNDYDFVSAENADDDFKLSIYLPSAMDEPVVYNGKKADIADADVFEKWLQVEALPYFGEIDGSVFAQYVESGLPLGYLFYNDEEELEEYKPLFTELAKKNRGLMNFVSIDARKFGRHAGNLNMKEQFPLFAIHDMTEDLKYGLPQLSEEAFDELSDKIVLESKAIESLVKDFLKGDASPIVKSQEIFENQDSSVFQLVGKNHDEIVNDPKKDVLVLYYAPWCGHCKRLAPTYQELADTYANATSDVLIAKLDHTENDVRGVVIEGYPTIVLYPGGKKSESVVYQGSRSLDSLFDFIKENGHFDVDGKALYEEAQEKAAEEADADAELADEEDAIHDEL

SEQ ID NO: 51 - DNA, 957 bp, Saccharomyces cerevisiae
  CDS (1)..(957)
atgttatttcttaatattattaagctccttttgggactttttattatgaatgaagtaaaggcgcaaaacttttacgattccgatcctcatatatcagagttaacgccaaaaagcttcgataaagcgatccataacacaaattacacatcattagtggaattttatgctccgtggtgcggccattgtaagaagctctctagtacgttccgcaaggcagcaaaaagattggatggtgtagtccaagttgctgctgtaaactgtgaccttaacaagaataaggctttgtgtgctaaatacgacgtaaacggatttcccacgttaatggtatttaggcccccaaaaattgacctatctaagccaatagataacgccaaaaaaagtttcagcgctcatgccaatgaagtgtactcaggtgcaagaactctcgcgcctattgttgatttttctctttcaagaataaggtcatatgtcaaaaagtttgtccgtatagatacacttggctctttacttagaaagtcacccaaactttccgtggtgttgttttccaaacaagacaaaatttcaccggtttataaaagcattgcccttgattggttaggaaagttcgatttttattcaatttcaaacaaaaaactcaagcaactaaccgatatgaacccaacatatgaaaaaactcctgagattttcaaatatttgcagaaggtcattcctgaacagcgacagagcgataaaagtaagcttgtcgtttttgatgcagacaaagataaattttgggagtatgaagggaactcaatcaacaaaaatgacatttccaaatttctgcgggacacttttagtattacccccaatgagggtccttttagtagacgttctgaatatattgcttacttaaaaactggcaagaagccaattaaaaagaaccattcctcctcaggaaacaagcacgacgaattgtag

SEQ ID NO: 52 - protein, 318 aa, Saccharomyces cerevisiae
MLFLNIIKLLLGLFIMNEVKAQNFYDSDPHISELTPKSFDKAIHNTNYTSLVEFYAPWCGHCKKLSSTFRKAAKRLDGVVQVAAVNCDLNKNKALCAKYDVNGFPTLMVFRPPKIDLSKPIDNAKKSFSAHANEVYSGARTLAPIVDFSLSRIRSYVKKFVRIDTLGSLLRKSPKLSVVLFSKQDKISPVYKSIALDWLGKFDFYSISNKKLKQLTDMNPTYEKTPEIFKYLQKVIPEQRQSDKSKLVVFDADKDKFWEYEGNSINKNDISKFLRDTFSITPNEGPFSRRSEYIAYLKTGKKPIKKNHSSSGNKHDEL

SEQ ID NO: 53 - DNA, 1134 bp, Saccharomyces cerevisiae
  CDS (1)..(1134)
atgattccaaaattatatatacatttgatactatctttattgttgttgccgctaattttggcgcaggattattatgcaatactagagatagacaaagatgccactgagaaggaaatcaaatcagcgtacagacaattgtctaagaagtaccatccggataaaaatgctgggagcgaagaagcccatcaaaaattcattgaagtcggcgaggcatacgatgtattgagcgatcctgaaaagaaaaagatttatgaccagtttggtgcagatgctgtaaagaatggcggtggcggtggcggtccaggaggccctggcgcaggtggattccacgatccgtttgacatattcgaacggatgtttcaaggaggtcatggaggtcctggcggcggatttggccagagacagaggcagcgtggtccaatgatcaaggtccaggaaaaactatctttaaagcagttttattccgggtcctcgatagaatttactttaaacctaaacgatgaatgtgatgcatgccatggtagtggctctgcagatggtaagctggcccaatgtcccgattgtcaaggtcgtggggttataatacaagtgctgcgcatgggtattatgacgcagcagattcaacagatgtgtggtaggtgtggtggtacgggacaaattatcaaaaatgaatgcaaaacatgtcacggcaaaaaagttaccaaaaagaacaagttcttccacgttgacgttccaccaggcgcaccaagaaactacatggacacaagagtcggcgaggctgaaaaagggcctgactttgacgccggtgacttggtcatagaattcaaggaaaaggatactgagaacatgggttacagaagaagaggcgacaatctgtacagaacagaagttctttctgctgcggaagcgctatacggcggatggcaaagaacgatagaattccttgatgagaacaagcccgttaagttatctagacccgctcatgtagttgtctccaatggcgaagttgaagtcgtgaagggattcggcatgcccaagggtagcaagggttacggtgatttgtacatagactacgtcgttgtcatgccaaagactttcaaatctgggcaaaatatgctcaaagatgagttgtag

SEQ ID NO: 54 - protein, 377 aa, Saccharomyces cerevisiae
MIPKLYIHLILSLLLLPLILAQDYYAILEIDKDATEKEIKSAYRQLSKKYHPDKNAGSEEAHQKFIEVGEAYDVLSDPEKKKIYDQFGADAVKNGGGGGGPGGPGAGGFHDPFDIFERMFQGGHGGPGGGFGQRQRQRGPMIKVQEKLSLKQFYSGSSIEFTLNLNDECDACHGSGSADGKLAQCPDCQGRGVIIQVLRMGIMTQQIQQMCGRCGGTGQIIKNECKTCHGKKVTKKNKFFHVDVPPGAPRNYMDTRVGEAEKGPDFDAGDLVIEFKEKDTENMGYRRRGDNLYRTEVLSAAEALYGGWQRTIEFLDENKPVKLSRPAHVVVSNGEVEVVKGFGMPKGSKGYGDLYIDYVVVMPKTFKSGQNMLKDEL

SEQ ID NO: 55 - DNA, 1692 bp, Saccharomyces cerevisiae
  CDS (1)..(1692)
atgagattaagaaccgccattgccacactgtgcctcacggcttttacatctgcaacttcaaacaatagctacatcgccaccgaccaaacacaaaatgcctttaatgacactcacttttgtaaggtcgacaggaatgatcacgttagtcccagttgtaacgtaacattcaatgaattaaatgccataaatgaaaacattagagatgatctttcggcgttattaaaatctgatttcttcaaatactttcggctggatttatacaagcaatgttcattttgggacgccaacgatggtctgtgcttaaaccgcgcttgctctgttgatgtcgtagaggactgggatacactgcctgagtactggcagcctgagatcttgggtagtttcaataatgatacaatgaaggaagcggatgatagcgatgacgaatgtaagttcttagatcaactatgtcaaaccagtaaaaaacctgtagatatcgaagacaccatcaactactgtgatgtaaatgactttaacggtaaaaacgccgttctgattgatttaacagcaaatccggaacgatttacaggttatggtggtaagcaagctggtcaaatttggtctactatctaccaagacaactgttttacaattggcgaaactggtgaatcattggccaaagatgcattttatagacttgtatccggtttccatgcctctatcggtactcacttatcaaaggaatatttgaacacgaaaactggtaaatgggagcccaatctggatttgtttatggcaagaatcgggaactttcctgatagagtgacaaacatgtatttcaattatgctgttgtagctaaggctctctggaaaattcaaccatatttaccagaattttcattctgtgatctagtcaataaagaaatcaaaaacaaaatggataacgttatttcccagctggacacaaaaatttttaacgaagacttagtttttgccaacgacctaagtttgactttgaaggacgaattcagatctcgcttcaagaatgtcacgaagattatggattgtgtgcaatgtgatagatgtagattgtggggcaaaattcaaactaccggttacgcaactgccttgaaaattttgtttgaaatcaacgacgctgatgaattcaccaaacaacatattgttggtaagttaaccaaatatgagttgattgcactattacagactttcggtagattatctgaatctattgaatctgttaacatgttcgaaaaaatgtacgggaaaaggttaaacggttctgaaaacaggttaagctcattcttccaaaataacttcttcaacattttgaaggaggcaggcaaatcgattcgttacaccatagagaacatcaattccactaaagaaggaaagaaaaagactaacaattctcaatcacatgtatttgatgatttaaaaatgcccaaagcagaaatagttccaaggccctctaacggtacagtaaataaatggaagaaagcttggaatactgaagttaacaacgttttagaagcattcagatttatttatagaagctatttggatttacccaggaacatctgggaattatctttgatgaaggtatacaaattttggaataaattcatcggtgttgctgattacgttagtgaggagacacgagagcctatttcctataagctagatatacaataa

SEQ ID NO: 56 - protein, 563 aa, Saccharomyces cerevisiae
MRLRTAIATLCLTAFTSATSNNSYIATDQTQNAFNDTHFCKVDRNDHVSPSCNVTFNELNAINENIRDDLSALLKSDFFKYFRLDLYKQCSFWDANDGLCLNRACSVDVVEDWDTLPEYWQPEILGSFNNDTMKEADDSDDECKFLDQLCQTSKKPVDIEDTINYCDVNDFNGKNAVLIDLTANPERFTGYGGKQAGQIWSTIYQDNCFTIGETGESLAKDAFYRLVSGFHASIGTHLSKEYLNTKTGKWEPNLDLFMARIGNFPDRVTNMYFNYAVVAKALWKIQPYLPEFSFCDLVNKEIKNKMDNVISQLDTKIFNEDLVFANDLSLTLKDEFRSRFKNVTKIMDCVQCDRCRLWGKIQTTGYATALKILFEINDADEFTKQHIVGKLTKYELIALLQTFGRLSESIESVNMFEKMYGKRLNGSENRLSSFFQNNFFNILKEAGKSIRYTIENINSTKEGKKKTNNSQSHVFDDLKMPKAEIVPRPSNGTVNKWKKAWNTEVNNVLEAFRFIYRSYLDLPRNIWELSLMKVYKFWNKFIGVADYVSEETREPISYKLDIQ

SEQ ID NO: 57 - DNA, 408 bp, Saccharomyces cerevisiae
  CDS (1)..(408)
atgatgtttaatatttaccttttcgtcacttttttttccaccattcttgcaggttccctgtcagatttggaaatcggtattatcaagagaataccggtagaagattgcttaattaaggcaatgccaggtgataaagttaaggttcattatacaggatctttattagaatcgggaactgtatttgactcaagttattcaagaggctctcctatcgcttttgaacttggcgttggcagagtaattaaaggttgggatcaaggtgttgccggcatgtgcgttggcgaaaaaagaaagctgcaaattccaagttctttggcctacggagaaagaggtgtcccaggcgtcattcctccaagtgctgatttggtgtttgatgtcgaattggtagacgtgaaatcagccgcctag

SEQ ID NO: 58 - protein, 135 aa, Saccharomyces cerevisiae
MMFNIYLFVTFFSTILAGSLSDLEIGIIKRIPVEDCLIKAMPGDKVKVHYTGSLLESGTVFDSSYSRGSPIAFELGVGRVIKGWDQGVAGMCVGEKRKLQIPSSLAYGERGVPGVIPPSADLVFDVELVDVKSAA

SEQ ID NO: 59 - DNA, 1938 bp, Saccharomyces cerevisiae
  CDS (1)..(1938)
atgatactgatctcgggatactgtcttttagtgtatagcgttattttgccagtactgatatcggcttctaagttatgtgatttggctgagttacaacgattgaacaagaatttaaaagtagacactgaatccttgccaaaataccaatggatcgctgggcagttggaacaaaactgcatgactgcggatccagcaagtgaaaatatgtcagacgtaattcaactagccaatcaaatatactacaaaattgggctgatccaattatccaacgatcaacatctaagagctattaacacatttgaaaaaatcgtttttaatgaaacttacaaaggttcttttgggaagctggcggaaaagaggctacaagagctgtatgtcgattttgggatgtgggacaaggtgcatcagaaggatgatcagtatgcgaaatatctgtccttgaatgaaaccatcagaaacaaaatatcatccaaagacgtttctgtggaggaagatatttctgagctgctacgcataacgccgtacgatgttaacgtcctctccacgcacatcgatgttctttttcacaaactagctgaagaaattgacgtttcgttagctgctgctatcattttggattacgaaacaatcctcgacaagcatttggctagcttaagcatagatacaagactttcgattcattatgtcatatctgttttacagacctttgtacttaactcagatgcgtcgttcaatataagaaaatgcctttccattgatatggactatgataaatgtaaaaaactaagcctgactatttccaaattgaacaaggtgaatccatcaaaaagacagatcctggatccagcaacatatgcatttgagaacaaaaagtttagaagttgggatagaattattgaattttatttgaaggataagaagccatttattacaccaatgaaaattcttaacaaagatacaaactttaaaaacaactacttctttttagaggaaattatcaaacaattgatagaagacgttcaactgtcgagacctttggcaaaaaatttattcgaagatcccccaataaccgatggttttgtcaaaccaaaatcatactatcataccgattatctagtatacattgattccattctttgtcaggcttctagcatgagtccggacgtcaagagagctaaactggctgcgccgttctgtaaaaagagtttgaggcattcactaacactagaaacatggaaacactatcaggatgctaagtccgagcaaaaacctttacctgagacggtattgagtgatgtatggaattccaatcctcatttgctgatgtatatggtaaactcaatacttaataaaagtaggtctaaacctcattcacagttcaaaaagcaattatatgaccagataaacaaatttttccaagataacggcctctcagagtcgaccaatccatacgtgatgaagaacttccgattattacagaaacaattacaaacctataaagagcataaacatcggaatttcaaccagcaatatttccaacaacaacaacagcagcaacaacaccaacgacatcaagcacccccagcagcgcctaactacgacccaaaaaaggactattataaaattcttggagtatcgcctagtgctagttcgaaagaaataaggaaagcatatttaaatttaaccaaaaaataccacccagacaaaataaaggccaaccataacgacaaacaagaatcaattcacgaaactatgtcacaaatcaatgaagcgtacgaaacattaagtgatgacgataaaaggaaggaatacgatctttccagatcaaacccccgccgcaacacttttcctcaggggcctaggcaaaataacatgttcaaaaatccaggaagtggcttcccattcggaaatggctttaaaatgaattttgggctttga

SEQ ID NO: 60 - protein, 645 aa, Saccharomyces cerevisiae
MILISGYCLLVYSVILPVLISASKLCDLAELQRLNKNLKVDTESLPKYQWIAGQLEQNCMTADPASENMSDVIQLANQIYYKIGLIQLSNDQHLRAINTFEKIVFNETYKGSFGKLAEKRLQELYVDFGMWDKVHQKDDQYAKYLSLNETIRNKISSKDVSVEEDISELLRITPYDVNVLSTHIDVLFHKLAEEIDVSLAAAIILDYETILDKHLASLSIDTRLSIHYVISVLQTFVLNSDASFNIRKCLSIDMDYDKCKKLSLTISKLNKVNPSKRQILDPATYAFENKKFRSWDRIIEFYLKDKKPFITPMKILNKDTNFKNNYFFLEEIIKQLIEDVQLSRPLAKNLFEDPPITDGFVKPKSYYHTDYLVYIDSILCQASSMSPDVKRAKLAAPFCKKSLRHSLTLETWKHYQDAKSEQKPLPETVLSDVWNSNPHLLMYMVNSILNKSRSKPHSQFKKQLYDQINKFFQDNGLSESTNPYVMKNFRLLQKQLQTYKEHKHRNFNQQYFQQQQQQQQHQRHQAPPAAPNYDPKKDYYKILGVSPSASSKEIRKAYLNLTKKYHPDKIKANHNDKQESIHETMSQINEAYETLSDDDKRKEYDLSRSNPRRNTFPQGPRQNNMFKNPGSGFPFGNGFKMNFGL

SEQ ID NO: 61 - DNA, 2646 bp, Saccharomyces cerevisiae
  CDS (1)..(2646)
atgcgaaacgttttaaggcttttatttttaacagcttttgttgctatagggtctttagcagccgttttaggtgttgattacggtcagcaaaatatcaaggccattgtggtttctccgcaagccccattagaacttgtgctcacaccagaggcaaaacggaaggagatatctggtctttcgataaaaagattaccaggttatggaaaggatgatccgaatgggattgaaagaatctacggttccgctgttggcagtttagcaacaaggtttccccaaaacacattgttgcatttgaaaccgctacttgggaaatcactagaagatgaaaccactgtaactttgtattcaaaacaacaccccggtttagaaatggtatcaacaaatagaagtaccatagcctttttagttgataatgtggaatatccattggaagagttagtggcaatgaatgtccaagagattgccaatagagccaattcactgttgaaggatagagatgcaagaactgaggactttgtaaacaagatgagttttacaattcctgacttttttgaccaacatcaaaggaaagcacttttagatgccagttcaataaccacaggaatcgaagagacatatctggttagtgaagggatgtctgttgcagttaactttgtattaaagcagcgccaatttccaccaggtgaacagcagcattatatcgtatatgacatggggagcggttctattaaggcctcaatgttctctatattgcagccggaggacactactcagcccgttacaatagaatttgaaggatatgggtataatccacatctaggtggtgcaaagtttacaatggatattggcagtttgatagagaataagtttttggaaacacacccagccataagaactgatgaattgcacgctaatcccaaggccttagcaaaaatcaaccaagcagcagagaaggcaaagttaattttaagcgccaattctgaggcaagtattaacatagaatcactgatcaacgatattgatttccgtacttctataactagacaggaattcgaagaatttattgcagactcgttattggacattgtcaaacccataaatgacgctgttacaaaacaattcggtggctatggaacaaatttacctgagataaatggggtcattttggcgggaggctcttcccgaattcccattgtgcaggatcaattaatcaaactcgtatccgaagaaaaagtgttgagaaatgtcaatgctgatgaatcagctgtgaatggtgttgttatgagagggatcaagttatctaattcgtttaagaccaagccgttaaatgttgttgaccgttctgtaaatacttattcattcaaattatcaaacgaatctgaactgtatgatgtgttcacgcgcggaagtgcttatccaaacaaaacatctattttgacaaacacgactgattcgattcctaataattttaccattgacttatttgagaatggtaaattgttcgaaactatcacagttaattcaggagctataaagaattcatattcctctgataagtgctcgtcaggagttgcgtataacattactttcgacttgtccagtgatagattattctctattcaagaggttaactgcatttgtcagagcgaaaatgacataggtaactccaagcaaattaagaacaaaggcagccgtttggcttttacttctgaggatgttgagatcaaaaggctttctccttcagaacgttcgcgtttgcatgagcatatcaagttgctcgataaacaggataaggaaagatttcaattccaagaaaatttaaacgttcttgaaagtaacttgtatgatgctagaaacctgctaatggatgatgaagttatgcaaaatggaccaaaatcccaagtagaagagttatcggagatggttaaagtatatttggattggctcgaagatgcatcctttgatactgaccctgaggatatagttagcagaattagagaaattggaatattaaaaaagaaaatagaactttacatggattctgcaaaggaacctttgaactctcaacaatttaaaggaatgcttgaagaaggccataagttacttcaggctatagaaacccataagaataccgttgaagaatttttgagtcaatttgaaaccgagtttgcggataccatagataatgttagagaagaatttaaaaagattaagcaaccagcgtatgtgtcgaaggcgttatctacatgggaggaaaccttaacctcttttaaaaattccattagcgaaatagagaagttcctggcaaaaaacctatttggcgaagaccttcgtgaacatttatttgaaatcaaattacaatttgatatgtatcgtacgaaactagaggaaaaactgcgtttaataaaaagcggtgatgaaagtcgcttaaatgaaataaagaagttacatttaagaaacttccgcctacaaaagagaaaggaggaaaagttgaaaagaaagcttgaacaggaaaaaagcagaaacaacaatgaaacagaatcgacagtaatcaactcggctgacgataaaactactattgtcaatgacaagaccaccgagtcgaatccaagttctgaggaagacattttgcatgatgaattatag

SEQ ID NO: 62 - protein, 881 aa, Saccharomyces cerevisiae
MRNVLRLLFLTAFVAIGSLAAVLGVDYGQQNIKAIVVSPQAPLELVLTPEAKRKEISGLSIKRLPGYGKDDPNGIERIYGSAVGSLATRFPQNTLLHLKPLLGKSLEDETTVTLYSKQHPGLEMVSTNRSTIAFLVDNVEYPLEELVAMNVQEIANRANSLLKDRDARTEDFVNKMSFTIPDFFDQHQRKALLDASSITTGIEETYLVSEGMSVAVNFVLKQRQFPPGEQQHYIVYDMGSGSIKASMFSILQPEDTTQPVTIEFEGYGYNPHLGGAKFTMDIGSLIENKFLETHPAIRTDELHANPKALAKINQAAEKAKLILSANSEASINIESLINDIDFRTSITRQEFEEFIADSLLDIVKPINDAVTKQFGGYGTNLPEINGVILAGGSSRIPIVQDQLIKLVSEEKVLRNVNADESAVNGVVMRGIKLSNSFKTKPLNVVDRSVNTYSFKLSNESELYDVFTRGSAYPNKTSILTNTTDSIPNNFTIDLFENGKLFETITVNSGAIKNSYSSDKCSSGVAYNITFDLSSDRLFSIQEVNCICQSENDIGNSKQIKNKGSRLAFTSEDVEIKRLSPSERSRLHEHIKLLDKQDKERFQFQENLNVLESNLYDARNLLMDDEVMQNGPKSQVEELSEMVKVYLDWLEDASFDTDPEDIVSRIREIGILKKKIELYMDSAKEPLNSQQFKGMLEEGHKLLQAIETHKNTVEEFLSQFETEFADTIDNVREEFKKIKQPAYVSKALSTWEETLTSFKNSISEIEKFLAKNLFGEDLREHLFEIKLQFDMYRTKLEEKLRLIKSGDESRLNEIKKLHLRNFRLQKRKEEKLKRKLEQEKSRNNNETESTVINSADDKTTIVNDKTTESNPSSEEDILHDEL

SEQ ID NO: 63 - DNA, 834 bp, Saccharomyces cerevisiae
  CDS (1)..(834)
atgaaattgcacggctttttattttccgtattatcaacatgcgtcgtcattttaccagcgttggcctacagtgaagctgtcacgatggtcaagtcgattgagcagtacttcgatatctgcaataggaatgattcttacacaatgataaaatactacacttcttggtgccaacattgtaaaactctggccccagtatacgaagagcttggtgagctatacgccaaaaaagctaataaagatgataccccaattaacttccttgaagttaactgtgaattcttcgggccaactttatgtaccgacttgcctggatttccaataattgaactggtcaaacctcgtactaagcccttagttcttccgaagctcgattggtcgtctatgaaatttcatgaaagactatggcaaagaatcaagacgtggttcaacaatcctaagtaccaactggatacgtctagggttgttcgttttgaagggagtaggaacctaaagagtttaagcaactttatcgatactgtaagaagtaaagatacagaagaaagattcatagaacatattttcgatgattctaggaattgcaatgaagaattacgttctcaacagcttctgtgtaaagctggtaaagaatactactctgatactttatctaaattatacggtgacgtgaatgggctggaaaaggaaaggcgaagactagaagctttaattaagcaaaatggagatgacttgagtaaagaagttaaagaaaaactgaaaatcattcgtctacaattgagcctattatcacacatagaagaccagttagaagataccagtagtcatgacgagctttga

SEQ ID NO: 64 - protein, 277 aa, Saccharomyces cerevisiae
MKLHGFLFSVLSTCVVILPALAYSEAVTMVKSIEQYFDICNRNDSYTMIKYYTSWCQHCKTLAPVYEELGELYAKKANKDDTPINFLEVNCEFFGPTLCTDLPGFPIIELVKPRTKPLVLPKLDWSSMKFHERLWQRIKTWFNNPKYQLDTSRVVRFEGSRNLKSLSNFIDTVRSKDTEERFIEHIFDDSRNCNEELRSQQLLCKAGKEYYSDTLSKLYGDVNGLEKERRRLEALIKQNGDDLSKEVKEKLKIIRLQLSLLSHIEDQLEDTSSHDEL

SEQ ID NO: 65 - DNA, 888 bp, Saccharomyces cerevisiae
  CDS (1)..(888)
atgaacggttactggaaacctgcgttggttgtcctgggattggtatctctatcatatgcttttaccaccattgaaacagaaattttccaattacaaaatgaaataagtacgaaatatggcccagatatgaacttctacaagttcttgaagttacctaaactgcagaattctagtacaaaggagattacaaaaaacttaagaaagctatccaagaagtaccatccggataagaaccctaaataccgtaaattgtatgaaaggttaaacctcgctactcaaattctttcaaacagctctaatcgtaagatttatgattattatctacagaatggctttccaaactatgatttccataagggtggtttttatttttccagaatgaagcctaagacttggttcctgctggcctttatttggatagtcgttaatattgggcagtatatcatttctattattcaatatcgttctcaaagatcaagaattgaaaacttcatcagtcagtgtaaacaacaggatgataccaatggactaggcgtaaaacaactaacgtttaaacaacatgaaaaggatgagggtaaaagtttggttgtaaggtttagcgatgtctatgttgtagagcctgatggaagtgaaacactaatttcgccagataccttggataaaccttcagtaaagaactgtttgttttggagaatacctgcttcggtttggaacatgacgtttggcaaatctgttggtagcgcaggaaaagaagaaataataacggatagtaaaaagtatgatggtaaccaaacaaaaaaggggaacaaagtaaaaaagggttctgcaaagaaaggccaaaagaaaatggaattgcctaacggtaaagtgatctattcacgtaaatga

SEQ ID NO: 66 - protein, 295 aa, Saccharomyces cerevisiae
MNGYWKPALVVLGLVSLSYAFTTIETEIFQLQNEISTKYGPDMNFYKFLKLPKLQNSSTKEITKNLRKLSKKYHPDKNPKYRKLYERLNLATQILSNSSNRKIYDYYLQNGFPNYDFHKGGFYFSRMKPKTWFLLAFIWIVVNIGQYIISIIQYRSQRSRIENFISQCKQQDDTNGLGVKQLTFKQHEKDEGKSLVVRFSDVYVVEPDGSETLISPDTLDKPSVKNCLFWRIPASVWNMTFGKSVGSAGKEEIITDSKKYDGNQTKKGNKVKKGSAKKGQKKMELPNGKVIYSRK

SEQ ID NO: 67 - DNA, 1554 bp, Saccharomyces cerevisiae
  CDS (1)..(1554)
atgcaagtgaccacaagatttatatctgcgatagtctcgttttgcctgtttgcttctttcacgttggctgaaaacagcgcaagagctacgccgggatcagatttactcgttctaacagagaagaaatttaaatcattcatcgaatctcatccgttagtcctcgtcgagttttttgctccatggtgtttgcattctcagatcttacgccctcacttagaagaggccgcctctattttaaaggagcataacgtcccagttgttcaaattgattgtgaggctaacagtatggtttgcctgcaacaaactataaatacctacccaaccttgaaaatctttaaaaatggtcgtatttttgatggtcaagtctatcgcggtgtcaagatcaccgatgaaatcactcagtacatgattcagctatacgaggcttctgtcatttatttaaattccgaagatgaaatccaaccatacttggaaaatgcaactttaccagtagtaataaacagaggcttgacaggcttgaatgaaacgtatcaagaagtcgcactggaccttgctgaggattacgtctttttatcccttctagattcagaagataagtcattatcaatccacttgccaaacactacagaaccaattctgtttgatggaaatgtagactctttggtcggaaattccgttgctctaactcagtggttaaaagtggtaattttaccttactttaccgacatcgaacctgatctcttccccaagtacatttctagcaatttgccgttggcttacttcttttatacttctgaggaagaattggaagattacactgatcttttcacgcagttaggtaaggaaaatcgtggccaaataaatttcattgcattaaactctacaatgttcccacaccacgttagattcctaaatatgagagaacagttcccattatttgctatccataatatgatcaataatctgaaatatggtttaccacaactaccagaagaagagtacgcgaaattagaaaaaccacaaccactagacagagatatgatcgttcagttggtaaaagattaccgtgaaggtactgccaagccaattgttaagtcagaagagattccaaaagaacaaaagtccaatgtttataaaatagttgggaagacacatgacgacattgttcatgatgatgacaaggatgtccttgtcaaatattacgcgacatggtgtattcatagtaaaaggtttgcgcctatttacgaagaaattgcaaatgtcttagcatctgatgaatctgttcgcgataaaatcttgatcgccgaagtagattcaggggcaaatgatatcttaagttttcctgtgacaggatatccaaccattgctttgtatcctgccggaaataactctaagcctattatcttcaataaaattagaaatttggaagatgttttcgaatttatcaaggaatcaggtacacatcacattgacggccaggcaatttatgataaattgcaccaggccaaggattctgaagtgtctactgaagataccgtacatgatgaattataa

SEQ ID NO: 68 - protein, 517 aa, Saccharomyces cerevisiae
MQVTTRFISAIVSFCLFASFTLAENSARATPGSDLLVLTEKKFKSFIESHPLVLVEFFAPWCLHSQILRPHLEEAASILKEHNVPVVQIDCEANSMVCLQQTINTYPTLKIFKNGRIFDGQVYRGVKITDEITQYMIQLYEASVIYLNSEDEIQPYLENATLPVVINRGLTGLNETYQEVALDLAEDYVFLSLLDSEDKSLSIHLPNTTEPILFDGNVDSLVGNSVALTQWLKVVILPYFTDIEPDLFPKYISSNLPLAYFFYTSEEELEDYTDLFTQLGKENRGQINFIALNSTMFPHHVRFLNMREQFPLFAIHNMINNLKYGLPQLPEEEYAKLEKPQPLDRDMIVQLVKDYREGTAKPIVKSEEIPKEQKSNVYKIVGKTHDDIVHDDDKDVLVKYYATWCIHSKRFAPIYEEIANVLASDESVRDKILIAEVDSGANDILSFPVTGYPTIALYPAGNNSKPIIFNKIRNLEDVFEFIKESGTHHIDGQAIYDKLHQAKDSEVSTEDTVHDEL

SEQ ID NO: 69 - DNA, 1527 bp, Homo sapiens
  CDS (1)..(1527)
atgctgcgccgcgctctgctgtgcctggccgtggccgccctggtgcgcgccgacgcccccgaggaggaggaccacgtcctggtgctgcggaaaagcaacttcgcggaggcgctggcggcccacaagtacctgctggtggagttctatgccccttggtgtggccactgcaaggctctggcccctgagtatgccaaagccgctgggaagctgaaggcagaaggttccgagatcaggttggccaaggtggacgccacggaggagtctgacctggcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggctgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccctggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacagcttccccacactcaagttctttcctgccagtgccgacaggacggtcattgattacaacggggaacgcacgctggatggttttaagaaattcctggagagcggtggccaggatggggcaggggatgatgacgatctcgaggacctggaagaagcagaggagccagacatggaggaagacgatgatcagaaagctgtgaaagatgaactgtaa

SEQ ID NO: 70 - protein, 508 aa, Homo sapiens
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 71 - DNA, 1407 bp, Homo sapiens
  CDS (1)..(1407)
atgggccgcggctggggattcttgtttggcctcctgggcgccgtgtggctgctcagctcgggccacggagaggagcagcccccggagacagcggcacagaggtgcttctgccaggttagtggttacttggatgattgtacctgtgatgttgaaaccattgatagatttaataactacaggcttttcccaagactacaaaaacttcttgaaagtgactactttaggtattacaaggtaaacctgaagaggccgtgtcctttctggaatgacatcagccagtgtggaagaagggactgtgctgtcaaaccatgtcaatctgatgaagttcctgatggaattaaatctgcgagctacaagtattctgaagaagccaataatctcattgaagaatgtgaacaagctgaacgacttggagcagtggatgaatctctgagtgaggaaacacagaaggctgttcttcagtggaccaagcatgatgattcttcagataacttctgtgaagctgatgacattcagtcccctgaagctgaatatgtagatttgcttcttaatcctgagcgctacactggttacaagggaccagatgcttggaaaatatggaatgtcatctacgaagaaaactgttttaagccacagacaattaaaagacctttaaatcctttggcttctggtcaagggacaagtgaagagaacactttttacagttggctagaaggtctctgtgtagaaaaaagagcattctacagacttatatctggcctacatgcaagcattaatgtgcatttgagtgcaagatatcttttacaagagacctggttagaaaagaaatggggacacaacattacagaatttcaacagcgatttgatggaattttgactgaaggagaaggtccaagaaggcttaagaacttgtattttctctacttaatagaactaagggctttatccaaagtgttaccattcttcgagcgcccagattttcaactctttactggaaataaaattcaggatgaggaaaacaaaatgttacttctggaaatacttcatgaaatcaagtcatttcctttgcattttgatgagaattcattttttgctggggataaaaaagaagcacacaaactaaaggaggactttcgactgcattttagaaatatttcaagaattatggattgtgttggttgttttaaatgtcgtctgtggggaaagcttcagactcagggtttgggcactgctctgaagatcttattttctgagaaattgatagcaaatatgccagaaagtggacctagttatgaattccatctaaccagacaagaaatagtatcattattcaacgcatttggaagaatttctacaagtgtgaaagaattagaaaacttcaggaacttgttacagaatattcattaa

SEQ ID NO: 72 - protein, 468 aa, Homo sapiens
MGRGWGFLFGLLGAVWLLSSGHGEEQPPETAAQRCFCQVSGYLDDCTCDVETIDRFNNYRLFPRLQKLLESDYFRYYKVNLKRPCPFWNDISQCGRRDCAVKPCQSDEVPDGIKSASYKYSEEANNLIEECEQAERLGAVDESLSEETQKAVLQWTKHDDSSDNFCEADDIQSPEAEYVDLLLNPERYTGYKGPDAWKIWNVIYEENCFKPQTIKRPLNPLASGQGTSEENTFYSWLEGLCVEKRAFYRLISGLHASINVHLSARYLLQETWLEKKWGHNITEFQQRFDGILTEGEGPRRLKNLYFLYLIELRALSKVLPFFERPDFQLFTGNKIQDEENKMLLLEILHEIKSFPLHFDENSFFAGDKKEAHKLKEDFRLHFRNISRIMDCVGCFKCRLWGKLQTQGLGTALKILFSEKLIANMPESGPSYEFHLTRQEIVSLFNAFGRISTSVKELENFRNLLQNIH

SEQ ID NO: 73 - DNA, 1404 bp, Homo sapiens
  CDS (1)..(1404)
atgagccaaggggtccgccgggcaggcgctgggcagggggtagcggccgcggtgcagctgctggtcaccctgagcttcctgcggagcgtcgtcgaggcgcaggtcactggagttctggatgattgcttgtgtgatattgacagcatcgataacttcaatacctacaaaatcttccccaaaataaaaaaattgcaagagagagactattttcgttattacaaggttaatctgaagcgaccttgtcctttctgggcagaagatggccactgttcaataaaagactgtcatgtggagccctgtccagagagtaaaattccggttggaataaaagctgggcattctaataagtacttgaaaatggcaaacaataccaaagaattagaagtttgtgagcaagctaataaactgggagcaattaacagcacattaagtaatcaaagcaaagaagctttcattgactgggcaagatatgatgattcacgggatcacttttgtgaacttgatgatgagagatctccagctgctcagtatgtagacctattgctgaacccagagcgttacactggctataaagggacctctgcatggagagtgtggaacagcatctatgaagagaactgtttcaagcctcgatctgtttatcgtcctttaaatcctctggcgcctagccgaggcgaagatgatggagaatcattctacacatggctagaaggtttgtgtctggagaaaagagtcttctataagcttatatcgggacttcatgctagcatcaatttacatctatgcgcaaattatcttttggaagaaacctggggtaagcccagttggggacctaatattaaagaattcaaacaccgctttgaccctgtggaaaccaagggagaaggtccaagaaggctcaagaatctttactttttatacttgattgagcttcgagctttgtcaaaggtggctccatattttgagcgctcaattgtcgatctttacactggaaatgcagaagaagatgctgacacaaaaactcttctactgaatatctttcaagatacaaagtcctttcccatgcactttgatgagaaatccatgtttgcaggtgacaaaaaaggggccaagtcactaaaggaggaattccgattacatttcaagaatatctcccgtataatggactgtgttggatgtgacaaatgcagattatggggaaaattacagactcagggtttaggaactgccctgaagatattattctctgaaaaagaaatccaaaagcttccagagaatagtccatctaaaggcttccaactcacccgacaggaaatagttgctcttttaaatgcttttggaaggctttctacaagtataagagacttacagaattttaaagtcttattacaacacagtaggtaa

SEQ ID NO: 74 - protein, 467 aa, Homo sapiens
MSQGVRRAGAGQGVAAAVQLLVTLSFLRSVVEAQVTGVLDDCLCDIDSIDNFNTYKIFPKIKKLQERDYFRYYKVNLKRPCPFWAEDGHCSIKDCHVEPCPESKIPVGIKAGHSNKYLKMANNTKELEVCEQANKLGAINSTLSNQSKEAFIDWARYDDSRDHFCELDDERSPAAQYVDLLLNPERYTGYKGTSAWRVWNSIYEENCFKPRSVYRPLNPLAPSRGEDDGESFYTWLEGLCLEKRVFYKLISGLHASINLHLCANYLLEETWGKPSWGPNIKEFKHRFDPVETKGEGPRRLKNLYFLYLIELRALSKVAPYFERSIVDLYTGNAEEDADTKTLLLNIFQDTKSFPMHFDEKSMFAGDKKGAKSLKEEFRLHFKNISRIMDCVGCDKCRLWGKLQTQGLGTALKILFSEKEIQKLPENSPSKGFQLTRQEIVALLNAFGRLSTSIRDLQNFKVLLQHSR

SEQ ID NO: 75 - DNA, 1965 bp, Homo sapiens
  CDS (1)..(1965)
atgaagctctccctggtggccgcgatgctgctgctgctcagcgcggcgcgggccgaggaggaggacaagaaggaggacgtgggcacggtggtcggcatcgacctggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaagacaatagagctgtgcagaaactccggcgcgaggtagaaaaggccaaacgggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtag

SEQ ID NO: 76 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL